(19) United States
(12) Patent Application Publication
(10) Pub. No.: US 2015/0065499 A1
(43) Pub. Date: Mar. 5, 2015

(54) 3-AMINOTHIENO[3,2-c]QUINOLINE DERIVATIVES, METHODS OF PREPARATION AND USES

(71) Applicant: TEMPLE UNIVERSITY - OF THE COMMONWEALTH SYSTEM OF HIGHER EDUCATION, PHILADELPHIA, PA (US)

(72) Inventors: E. Premkumar Reddy, Villanova, PA (US); M. V. Ramana Reddy, Upper Darby, PA (US)

(21) Appl. No.: 14/384,254

(22) PCT Filed: Feb. 26, 2013
(86) PCT No.: PCT/US13/27778

§ 371 Date: Sep. 10, 2014

Related U.S. Application Data
(60) Provisional application No. 61/613,042, filed on Mar. 20, 2012.

(51) Int. Cl. C07D 495/04

(52) U.S. Cl. CPC C07D 495/04
USPC 514/232.8; 546/80; 514/291; 544/126; 544/361; 514/253.03

(57) ABSTRACT

The present invention relates to compounds according to Formula I: and salts thereof, wherein R₁, R₂, R₃, R₄, R₅, R₆, R₇, X, and Y are as defined herein. Methods for preparing compounds of Formula I are also provided. The present invention further includes methods of treating cellular proliferative disorders, such as cancer, with the compounds of Formula I.

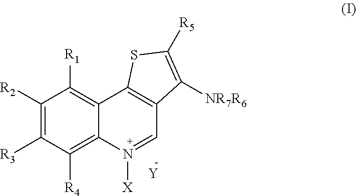

### CROSS REFERENCE TO RELATED APPLICATIONS

This application claims the benefit of U.S. Provisional Application No. 61/613,042, filed Mar. 20, 2012, the entire disclosure of which is incorporated herein by reference.

### FIELD OF THE INVENTION

The invention relates to compounds, methods for their preparation, compositions including them. The invention further provides methods for the treatment of cellular proliferative disorders, including, but not limited to, cancer.

### BACKGROUND OF THE INVENTION

Cellular proliferative disorders such as cancer are among the most common causes of death in developed countries. That said, many cellular proliferative disorders have no available cures or few, if any, treatment options to slow the progression of the disease. For cellular proliferative diseases for which treatments exist, undesirable side effects and limited efficacy often call into question the utility of a given treatment. This is particularly true when the available treatment option(s) may not appreciably prolong life, but have a definitive adverse effect on the quality of time remaining. Thus, identifying new effective drugs for cellular proliferative disorders, and in particular cancer, is a continuing focus of medical research.

Phosphatidylinositol 3-kinase (PI3K), a lipid kinase, is a key component of the PI3K/Akt/mTOR signaling pathway that controls cell growth, proliferation and survival, anabolic and autophagic activities, and cytoskeletal organization (Engelman et al., Nat Rev Genet. 2006; 7: 606-19). The PI3K/Akt/mTOR pathway responds to multiple inputs including growth signals as well as metabolic and nutritional cues (Engelman et al., supra; Vivanco and Sawyers, Nature Rev. Cancer, 2002, 2, 489-501). Four distinct isoforms of PI3K are known with different expression patterns and different pathophysiological roles: α, β, δ and γ. The PI3Ks function as dimeric complexes of a catalytic subunit p110α, p110β, p110δ or p110γ, with regulatory protein subunit p85, p101 or p84. All four isoforms of PI3K appear to be implicated to varying degrees in the development of cancer and are often upregulated in cancer cells but differ in their cellular expression (Jia et al., Curr. Opin. Cell. Biol., 2009, 21, 199-208).

PI3 Kinases have emerged as one of the most extensively studied classes of therapeutic targets in small molecule drug discovery particularly in oncology, based on at least the following observations:
(1) The phosphatase PTEN, the negative regulator of PI3K, is one of the most commonly mutated proteins in human malignancy (Vogt and Hart, Curr. Opin. Genetics and Dev. 2009, 19, 1-6).
(2) The gene encoding for the p110α subunit, PIK3CA, is amplified, overexpressed and frequently mutated in many cancers (Billottet et al., Oncogene, 2006, 25, 6648-6659); critically, these mutations have been shown to reduce cellular dependence on growth factors, to attenuate apoptosis, and to facilitate tumor invasiveness.
(3) PI3Kβ has been shown to be significant in certain cancers and also in cardiac regulation and thrombosis, and is also activated via coupling to GPCRs in contrast to the other three PI3K isoforms (Fruman and Rommel, Cancer Disc. 2011; 10: 562-572).
(4) Studies have shown that p110α is critical for the growth of tumors driven by PIK3CA mutations as well as oncogenic receptor tyrosine kinases and RAS, while p110β is the principal isoform involved in mediating PTEN-deficient tumorigenesis (Billottet et al., supra).
(5) PI3Kδ□ is upregulated in blast cells in patients with acute myeloid leukemia, where it plays a key role in cell survival highlighting its potential as a target in leukemia and other hematological malignancies. There is also some evidence that this isoform is upregulated in melanoma and breast cancer, and is overexpressed in neuroblastoma (Fruman and Rommel, supra).
(6) PI3Kδ subtype has also been shown to play a central function in the recruitment and activation of a range of immune and inflammatory cells suggesting its importance for immune and inflammatory diseases (Fruman and Rommel, supra).

PI3Kγ is expressed at highest levels in leukocytes. It is also detectable in certain other cell types. Attention has been given to PI3Kγ as a target in inflammatory diseases driven by leukocytes (Marone et al., Biochim Biophys Acta 2008; 1784:159-8516; Ruckle et al., Nat Rev Drug Discov 2006; 5:903-18). PI3Kγ is essential for myeloid cell adhesion to endothelium and extravasation into tumor sites in mice (Schmid et al., Cancer Cell 2011; 19:715-27.). Pharmacological or genetic blockade of PI3Kγ suppressed inflammation, growth, and metastasis of implanted and spontaneous tumors, indicating that PI3Kγ is a therapeutic target in oncology (Schmid et al., supra).

### SUMMARY OF THE INVENTION

It has been found that certain compounds and compositions are useful for the treatment of cancer and other cellular proliferative disorders. The biologically active compounds of the invention are 3-aminothieno[3,2-c]quinoline derivatives and related derivatives thereof.

Provided is a compound of Formula I or a salt thereof wherein:

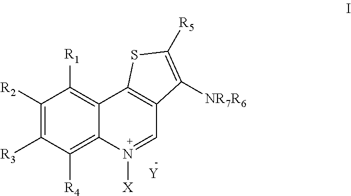

wherein,
R₁ is selected from the group consisting of: H; halogen; nitro; cyano; —O—R₈; —O—C(═O)—(CH₂)ₙR₉; —C(═O)—R₁₀; —NR₁₁R₁₂; and a saturated or unsaturated, straight or branched, cyclic or acyclic, chiral or achiral hydrocarbyl group having from 1 to 10 carbon atoms, wherein the hydrocarbyl group is optionally substituted with one or more of hydroxy, (C₁-C₄)alkoxy, amino, (C₁-C₄)alkylamino, (C₁-C₄)dialkylamino, halogen, cyano, nitro, carboxy, (C₁-C₄)alkoxycarbonyl or sulfonamido;
R₂ is selected from the group consisting of: H; halogen; nitro; cyano; —O—R₈; —O—C(═O)—(CH₂)ₙR₉; —C(═O)—R₁₀; —NR₁₁R₁₂; and a saturated or unsaturated, straight or branched, cyclic or acyclic, chiral or achiral hydrocarbyl group having from 1 to 10 carbon atoms, wherein the hydrocarbyl group is optionally substituted with one or more of hydroxy, (C₁-C₄)alkoxy, amino, (C₁-C₄)alkylamino, (C₁-C₄)dialkylamino, halogen, cyano, nitro, carboxy, (C₁-C₄)alkoxycarbonyl or sulfonamido;
R₃ is selected from the group consisting of H; halogen; nitro; cyano; —O—R₈; —O—C(═O)—(CH₂)ₙR₉; —C(═O)—R₁₀; —NR₁₁R₁₂; and a saturated or unsaturated, straight or branched, cyclic or acyclic, chiral or achiral hydrocarbyl group having from 1 to 10 carbon atoms, wherein the hydrocarbyl group is optionally substituted with one or more of hydroxy, (C₁-C₄)alkoxy, amino, (C₁-C₄)alkylamino, (C₁-C₄)dialkylamino, halogen, cyano, nitro, carboxy, (C₁-C₄)alkoxycarbonyl or sulfonamido;
R₄ is selected from the group consisting of: H; halogen; nitro; cyano; —O—R₈; —O—C(═O)—(CH₂)ₙR₉; —C(═O)—R₁₀; —NR₁₁R₁₂; and a saturated or unsaturated, straight or branched, cyclic or acyclic, chiral or achiral hydrocarbyl group having from 1 to 10 carbon atoms, wherein the hydrocarbyl group is optionally substituted with one or more of hydroxy, (C₁-C₄)alkoxy, amino, (C₁-C₄)alkylamino, (C₁-C₄)dialkylamino, halogen, cyano, nitro, carboxy, (C₁-C₄)alkoxycarbonyl or sulfonamido;
R₅ is selected from the group consisting of CN; NO₂; (C₁-C₆)perfluoroalkyl; substituted or unsubstituted (C₆-C₁₀)aryl; substituted or unsubstituted (C₂-C₉)heterocyclyl; substituted or unsubstituted (C₇-C₁₅)aralkyl; substituted or unsubstituted (C₂-C₉)heterocyclylalkyl; and —C(═O)—R₁₃;
R₆ is selected from the group consisting of H; a saturated or unsaturated, straight or branched, cyclic or acyclic, chiral or achiral hydrocarbyl group having from 1 to 10 carbon atoms, wherein the hydrocarbyl group is substituted with (C₁-C₆)carboxy, (C₁-C₆)carboxyalkyl, hydroxy, or (C₁-C₆) alkoxy; and substituted or unsubstituted (C₁₁-C₁₅)aralkyl;
R₇ is selected from the group consisting of H; saturated or unsaturated, straight or branched, cyclic or acyclic, chiral or achiral hydrocarbyl group having from 1 to 10 carbon atoms, wherein the hydrocarbyl group is substituted with (C₁-C₆)carboxy, (C₁-C₆)alkoxycarbonyl, hydroxy, or (C₁-C₆) alkoxy; substituted or unsubstituted (C₇-C₁₅)aralkyl; and —C(═O)—R₁₄;
or
R₆ and R₇ combine to form ═CH—NR₁₇R₁₈;
R₈ is independently selected from the group consisting of:

H;

a saturated or unsaturated, straight or branched, cyclic or acyclic, chiral or achiral hydrocarbyl group having from 1 to 10 carbon atoms, wherein the hydrocarbyl group is optionally substituted with one or more of halogen, hydroxy, (C₁-C₄)alkylcarbonyloxy, or —NR₁₁R₁₂, and wherein one or more of the carbon atoms in the hydrocarbyl group is optionally replaced by an oxygen atom (—O—);

substituted or unsubstituted (C₆-C₁₀)aryl;

substituted or unsubstituted (C₂-C₉)heterocyclyl; and

—NH₂;

R₉ is independently selected from the group consisting of (C₁-C₆)alkyl; substituted or unsubstituted (C₆-C₁₀)aryl; substituted or unsubstituted (C₇-C₁₅)aralkyl; substituted or unsubstituted (C₂-C₉)heterocyclyl; substituted or unsubstituted (C₂-C₉)heterocyclylalkyl; and —NH₂;
R₁₀ is independently selected from the group consisting of —OR₁₅; —NR₁₁R₁₂, and a saturated or unsaturated, straight or branched, cyclic or acyclic, chiral or achiral hydrocarbyl group having from 1 to 10 carbon atoms, wherein the hydrocarbyl group is optionally substituted with one or more of halogens, hydroxy, alkoxy, cyano, carboxy, carbalkoxy, carboxamide, —NR₁₁R₁₂, or nitro;
R₁₁ and R₁₂ are each independently selected from the group consisting of H; (C₂-C₄)acyl; saturated or unsaturated, straight or branched, cyclic or acyclic, chiral or achiral hydrocarbyl group having from 1 to 10 carbon atoms; and substituted or unsubstituted (C₂-C₉)heterocyclyl;

wherein R₁₁ and R₁₂ may combine with O, S, or the nitrogen atom in NR₁₆ to form a substituted or unsubstituted heterocyclyl ring;

R₁₃ is selected from the group consisting of —OR₁₅; and a saturated or unsaturated, straight or branched, cyclic or acyclic, chiral or achiral hydrocarbyl group having from 1 to 10 carbon atoms, wherein the hydrocarbyl group is substituted with one or more of halogens, hydroxy, alkoxy, cyano, carboxy, carbalkoxy, carboxamido, or nitro;
R₁₄ is selected from the group consisting of:

substituted or unsubstituted (C₂-C₉)heterocyclyl;

—C(═O)-substituted or unsubstituted (C₆-C₁₀)aryl;

—C(═O)-substituted or unsubstituted (C₂-C₉)heterocyclyl;

—C(═O)—NH-substituted or unsubstituted (C₆-C₁₀)aryl;

—C(═O)—NH-substituted or unsubstituted (C₆-C₁₀)arylkyl;

—C(═O)—NH-substituted or unsubstituted (C₂-C₉)heterocyclyl;

—C(═O)-substituted or unsubstituted (C₂-C₉)heterocyclyl-substituted or unsubstituted (C₆-C₁₀)heteroaryl; and

—C(═O)—NH-substituted or unsubstituted (C₆-C₁₀)aryl-substituted or unsubstituted (C₂-C₉)heterocyclyl.

R₁₅ is independently selected from the group consisting of (C₁-C₆)alkyl; substituted or unsubstituted (C₉-C₁₅)aralkyl; substituted or unsubstituted (C₂-C₉)heterocyclyl; and substituted or unsubstituted (C₂-C₉)heterocyclylalkyl;
R₁₆ is selected from the group consisting of H; substituted or unsubstituted (C₁-C₆)alkyl; substituted or unsubstituted (C₆-C₁₀)aryl; and substituted or unsubstituted (C₇-C₁₅)aralkyl;
R₁₇ and R₁₈ are each independently (C₁-C₆)alkyl;
n is independently selected from the group consisting of 0, 1, 2, 3, 4, and 5;
X is absent or selected from (C₁-C₆)alkyl and (C₁-C₆)alkoxyoxo(C₁-C₆)alkyl; and
Y is absent or a counterion, preferably a halide,
i) provided that when R₆ is H, R₇ is H, and R₁₃ is —OR₁₅, then at least one of R₂ and R₃ is —O—R₈, wherein R₈ is selected from the group consisting of: H; a saturated or unsaturated, straight or branched, cyclic or acyclic, chiral or achiral hydrocarbyl group having from 1 to 10 carbon atoms, wherein the hydrocarbyl group is substituted with one or more of halogen, hydroxy, (C₁-C₄)alkylcarbonyloxy, or —NR₁₁R₁₂; substituted or unsubstituted (C₆-C₁₀)aryl; substituted or unsubstituted (C₂-C₉)heterocyclyl; and —NH₂;
ii) provided that when X is absent, then the N⁺ of Formula I is N and Y is absent, and
iii) when X is (C₁-C₆)alkyl or (C₁-C₆)alkoxyoxo(C₁-C₆)alkyl, then Y is a counterion.

The present disclosure further provides a pharmaceutical composition comprising a pharmaceutically acceptable carrier and a compound of Formula I or Formula II, or a pharmaceutically acceptable salt thereof.

The present disclosure further provides a method of treating an individual suffering from a cellular proliferative disorder, comprising administering to the individual an effective amount of at least one compound of Formula I or Formula II, or a pharmaceutically acceptable salt thereof.

In particular embodiments, the cellular proliferative disorder is selected from the group consisting of cancer, malignant and benign tumors, blood vessel proliferative disorders, autoimmune disorders, and fibrotic disorders.

In particular embodiments, the cellular proliferative disorder is selected from the group consisting of hemangiomatosis in newborn, secondary progressive multiple sclerosis, atherosclerosis, chronic progressive myelodegenerative disease, neurofibromatosis, ganglioneuromatosis, keloid formation, Paget's disease of the bone, fibrocystic disease of the breast, uterine fibroids, Peyronie's disease, Dupuytren's disease, restenosis, benign proliferative breast disease, benign prostatic hyperplasia, X linked lymphocellular proliferative disorder, post transplantation lymphocellular proliferative disorder, macular degeneration, retinopathies, proliferative vitreoretinopathy, non cancerous lymphocellular proliferative disorders, and cancer.

In particular embodiments, the cellular proliferative disorder is cancer. In certain embodiments, the cancer is selected from the group consisting of ovarian cancer; cervical cancer; breast cancer; prostate cancer; testicular cancer, lung cancer, renal cancer; colorectal cancer; skin cancer; brain cancer; leukemia, including acute myeloid leukemia, chronic myeloid leukemia, acute lymphoid leukemia, and chronic lymphoid leukemia.

The present disclosure further provides a method of inducing apoptosis of cancer cells in an individual afflicted with cancer, comprising administering to the individual an effective amount of at least one compound of Formula I or Formula II, or a pharmaceutically acceptable salt thereof.

In certain embodiments, the cancer cells are tumor cells. In particular embodiments, the tumor cells are selected from the group consisting of ovarian, cervical, breast, prostate, testicular, lung, renal, colorectal, skin and brain tumor cells.

In certain embodiments, the invention is a compound of Formula I or Formula II, or salt thereof, for use in medicine. In other embodiments, the invention is a compound of Formula I or Formula II or a salt thereof, for treatment of a cellular proliferative disorder. In other embodiments, the invention provides a use of a compound according to Formula I or Formula II, or a salt thereof, for preparation of a medicament for treatment of a cellular proliferative disorder. The present invention further provides a medicament for treatment of a cellular proliferative disorder, containing a compound of Formula I or Formula II.

The present disclosure further provides a method of treating an individual suffering from a cellular proliferative disorder, comprising administering to the individual an effective amount of at least one compound of Formula II, or a pharmaceutically acceptable salt thereof wherein:

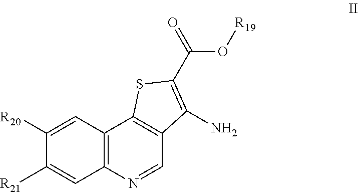

R₁₉ is selected from the group consisting of: (C₁-C₆)alkyl; substituted or unsubstituted (C₇-C₁₅)aralkyl; and substituted or unsubstituted (C₆-C₁₀)aryl; and
R₂₀ and R₂₁ are each independently selected from the group consisting of H, halogen, and —O—CH₃.

As envisioned in the present invention with respect to the disclosed compositions of matter and methods, in one aspect the embodiments of the invention comprise the components and/or steps disclosed herein. In another aspect, the embodiments of the invention consist essentially of the components and/or steps disclosed herein. In yet another aspect, the embodiments of the invention consist of the components and/or steps disclosed herein.

### DETAILED DESCRIPTION OF THE INVENTION

The compounds and compositions of the invention are believed to selectively inhibit proliferation of cancer cells, and kill various tumor cell types without killing (or with reduced killing of) normal cells. Cancer cells are killed at concentrations where normal cells may be temporarily growth-arrested but not killed.

The compounds of the invention are believed to inhibit the proliferation of tumor cells, and for some compounds, induce cell death. Cell death results from the induction of apoptosis. The compounds are believed effective against a broad range of tumor types, including but not limited to the following: ovarian cancer, breast cancer, prostate cancer, lung cancer, renal cancer, colorectal cancer, brain cancer and leukemia.

The compounds are also believed useful in the treatment of non-cancer cellular proliferative disorders, including but not limited to the following: hemangiomatosis in newborn, secondary progressive multiple sclerosis, chronic progressive myelodegenerative disease, neurofibromatosis, ganglioneuromatosis, keloid formation, Paget's disease of the bone, fibrocystic disease of the breast, uterine fibroids, Peyronie's disease, Dupuytren's disease, restenosis and cirrhosis.

At least one compound of the invention, methyl 3-(4-fluorobenzamido)-7,8-dimethoxythieno[3,2-c]quinoline-2-carboxylate, selectively inhibits the activity of PI3Kγ, and thus is believed useful as an anti-inflammatory agent, particularly inflammatory diseases driven by leukocytes. Apart from inhibiting tumor cell proliferation, the PI3Kγ inhibitory activity of the compound provides further anticancer utility by blocking PI3Kγ-induced myeloid cell adhesion to endothelium and extravasation, and thus inhibition of angiogenesis. See, Schmid et al., Cancer Cell 2011; 19:715-27, demonstrating that pharmacological or genetic blockade of PI3Kγ suppresses inflammation, growth, and metastasis of implanted and spontaneous tumors.

### I. DEFINITIONS

### 1. General

As used in the specification and the appended claims, the singular forms “a,” “an” and “the” include plural referents unless the context clearly dictates otherwise.

As used herein, the terms “treat” and “treatment” are used interchangeably and are meant to indicate a postponement of development of a disorder and/or a reduction in the severity of symptoms that will or are expected to develop. The terms further include ameliorating existing symptoms, preventing additional symptoms, and ameliorating or preventing the underlying metabolic causes of symptoms.

As used herein, “individual” (as in the subject of the treatment) means both mammals and non-mammals. Mammals include, for example, humans; non-human primates, e.g. apes and monkeys; cattle; horses; sheep; and goats. Non-mammals include, for example, fish and birds.

The expression “effective amount”, when used to describe therapy to an individual suffering from a cancer or other cellular proliferative disorder, refers to the amount of a compound according to Formula I or Formula II that inhibits the abnormal growth or proliferation, or alternatively induces apoptosis of cancer cells, preferably tumor cells, resulting in a therapeutically useful and selective cytotoxic effect on proliferative cells.

The term “cellular proliferative disorder” means a disorder wherein unwanted cell proliferation of one or more subsets of cells in a multicellular organism occurs. In some such disorders, cells are made by the organism at an atypically accelerated rate.

### 2. Chemical

In the following paragraphs some of the definitions include examples. The examples are intended to be illustrative, and not limiting.

The term “alkyl”, by itself or as part of another substituent means, unless otherwise stated, a straight, or branched chain hydrocarbon having the number of carbon atoms designated (i.e. C₁-C₆ means one to six carbons) and includes straight, branched chain or cyclic groups. Examples include: methyl, ethyl, propyl, isopropyl, butyl, isobutyl, tert-butyl, pentyl, neopentyl, and hexyl. Most preferred is (C₁-C₃)alkyl, particularly ethyl, methyl and isopropyl. Unless otherwise noted, an “alkyl” can refer to a (C₁-C₅)alkyl.

The term “alkoxy” employed alone or in combination with other terms means, unless otherwise stated, an alkyl group, as defined above, connected to the rest of the molecule via an oxygen atom, such as, for example, methoxy, ethoxy, 1-propoxy, 2-propoxy (isopropoxy) and the higher homologs and isomers. The alkyl portion of the alkoxy group can have a designated number of carbon atoms as defined for alkyl groups above. Preferred are (C₁-C₃)alkoxy, particularly ethoxy and methoxy.

The term “carboxy” means —C(═O)—O-J, wherein J can be H or a counter ion, including an alkaline metal.

The term “carboxyalkyl” means —C(═O)—O—(C₁-C₆)alkyl.

The term “carboxamido” means —C(═O)—NH₂.

The term “carbalkoxy” means —O—(C₁-C₆)alkyl-C(═O)—OJ, wherein J can be H or a counter ion, including an alkaline metal.

The terms “halo” or “halogen” by themselves or as part of another substituent mean, unless otherwise stated, a fluorine, chlorine, bromine, or iodine atom. Preferably, a halogen includes fluorine, chlorine, or bromine, more preferably, fluorine or chlorine.

The term “(Cₓ-C_{y})perfluoroalkyl,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkyl, more preferred is —(C₁-C₃)perfluoroalkyl, most preferred is —CF₃.

The term “aromatic” refers to a carbocycle or heterocycle having one or more polyunsaturated rings having aromatic character (i.e. having (4n+2) delocalized π (pi) electrons where n is an integer).

The term “aryl”, employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl; anthracyl; and naphthyl. Preferred are phenyl and naphthyl, most preferred is phenyl.

“Substituted aryl” means an aryl, as defined above, substituted by one, two, three, four, or five substituents. In some embodiments, the substituents are selected from among the group consisting of halogen, —OH, —NH₂, —N(CH₃)₂, —C(═O)OH, —C(═O)O(C₁-C₄)alkyl, trifluoromethyl, —O—(C₁-C₆)alkyl, —C(═O)NH₂, —SO₂NH₂, —C(═NH)NH₂, —C≡N and —NO₂. Preferably, a substituted aryl contains one or two substituents selected from halogen, —OH, NH₂, —O—(C₁-C₆)alkyl, trifluoromethyl, and —C(═O)OH, more preferably selected from halogen.

The term “(Cₓ-C_{y}) aralkyl”, wherein x<y, means a functional group wherein an aryl group is attached to the rest of the molecule through an alkylene chain, e.g., —CH₂CH₂-phenyl, the minimum number of the total carbon atoms in the alkyl group and aryl group is x, and the maximum number of the total carbon atoms in the alkyl and aryl groups is y. The term “substituted (Cₓ-C_{y}) aralkyl” means an (Cₓ-C_{y}) aralkyl group in which the aryl group is substituted. Preferred is substituted —CH₂CH₂-phenyl.

The term “heteroaryl” or “heteroaromatic” refers to a heterocycle having aromatic character. A polycyclic heteroaryl may include one or more rings which are partially saturated. Examples of heteroaryl groups include: pyridyl, pyrazinyl, pyrimidinyl, particularly 2- and 4-pyrimidinyl, pyridazinyl, thienyl, furyl, and pyrrolyl, preferably pyridyl.

The term “heterocycle” or “heterocyclyl” or “heterocyclic” by itself or as part of another substituent means, unless otherwise stated, an unsubstituted or substituted, stable, mono- or multi-cyclic heterocyclic ring system which consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom which affords a stable structure.

Examples of heterocycles (non-aromatic) include monocyclic groups such as: aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide, preferably piperidine, piperazine, and morpholine.

Examples of polycyclic heterocycles include: indolyl, particularly 3-, 4-, 5-, 6- and 7-indolyl, indolinyl, quinolyl, tetrahydroquinolyl, isoquinolyl, particularly 1- and 5-isoquinolyl, 1,2,3,4-tetrahydroisoquinolyl, cinnolinyl, quinoxalinyl, particularly 2- and 5-quinoxalinyl, quinazolinyl, phthalazinyl, 1,8-naphthyridinyl, 1,4-benzodioxanyl, coumarin, dihydrocoumarin, benzofuryl, particularly 3-, 4-, 1,5-naphthyridinyl, 5-, 6- and 7-benzofuryl, 2,3-dihydrobenzofuryl, 1,2-benzisoxazolyl, benzothienyl, particularly 3-, 4-, 5-, 6-, and 7-benzothienyl, benzoxazolyl, benzthiazolyl, particularly 2-benzothiazolyl and 5-benzothiazolyl, purinyl, benzimidazolyl, particularly 2-benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

The term “(Cₓ-C_{y}) heterocyclyl”, wherein x<y, means a functional group that is a heterocycle, wherein the minimum number of carbon atoms in the heterocycle is x, and the maximum number of carbon atoms in the heterocycle is y. The term “substituted (Cₓ-C_{y}) heterocyclyl” means a (Cₓ-C_{y}) heterocyclyl functional group in which the heterocyclyl group is substituted, preferably -piperazine-CH₃.

The term “(Cₓ-C_{y}) heterocyclylalkyl”, wherein x<y, means a functional group wherein an alkyl group is attached to a heterocycle group, e.g., —CH₂—CH₂-piperazine, the minimum number of the total carbon atoms in the alkyl and heterocycle is x, and the maximum number of the total carbon atoms in the alkyl and heterocycle is y. The term “substituted (Cₓ-C_{y}) heterocyclylalkyl” means a (Cₓ-C_{y}) heterocyclylalkyl functional group in which the heterocyclyl group is substituted, preferably —CH₂-piperizino-N-4-pyridine.

The term “hydrocarbyl” refers to any moiety comprising only hydrogen and carbon atoms, unless otherwise explicitly stated. For example, a hydrocarbyl optionally substituted with one or more halogen includes a perfluoroalkyl group, such as (C₁-C₆)perfluoroalkyl. A hydrocarbyl can also form a non-aromatic, such as cyclohexane, or an aromatic ring, such as a substituted or unsubstituted (C₆-C₁₀)aryl group. Preferred hydrocarbyl groups are (C₁-C₁₂)hydrocarbyl, more preferred are (C₁-C₇)hydrocarbyl, and most preferred are benzyl and (C₁-C₆) alkyl. For example, a hydrocarbyl group that is substituted by an oxygen include alkyoxy groups and alkyloxyalkyl groups, such as —O—CH₂—CH₃ or —CH₂—O—CH₃, respectively.

The phrase “—C(═O)-substituted or unsubstituted (C₂-C₉)heterocyclyl-substituted or unsubstituted (C₆-C₁₀)heteroaryl” means a carbonyl group attached to a substituted or unsubstituted (C₂-C₉)heterocyclyl, as defined above, which is further attached to a substituted or unsubstituted (C₆-C₁₀)heteroaryl, as defined above. An example of “—C(═O)-unsubstituted (C₂-C₉)heterocyclyl-unsubstituted (C₆-C₁₀)heteroaryl” is —C(═O)-piperizino-N-4-pyridine.

The phrase “—C(═O)—NH-substituted or unsubstituted (C₆-C₁₀) aryl-substituted or unsubstituted (C₂-C₉)heterocyclyl” means an amide group attached to a substituted or unsubstituted (C₆-C₁₀)aryl, as defined above, which is further attached to a substituted or unsubstituted (C₂-C₉)heterocyclyl, as defined above. For example, a “—C(═O)—NH-substituted or unsubstituted (C₆-C₁₀)aryl-substituted or unsubstituted (C₂-C₉)heterocyclyl” is —C(═O)—NH—[(N—CH₃-piperizino)benzyl].

The term “substituted” means that an atom or group of atoms has replaced hydrogen as the substituent attached to another group. For aryl and heteroaryl groups, the term “substituted” refers to any level of substitution, namely mono-, di-, tri-, tetra-, or penta-substitution, where such substitution is permitted. The substituents are independently selected, and substitution may be at any chemically accessible position.

Where a substituent is an alkyl or alkoxy group, the carbon chain may be branched, straight or cyclic, with straight being preferred.

### II. COMPOUNDS OF THE INVENTION

In one aspect, the invention is a compound of Formula I, or a salt thereof wherein:

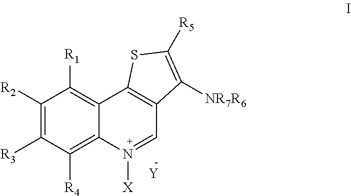

R₁ is selected from the group consisting of: H; halogen; nitro; cyano; —O—R₈; —O—C(═O)—(CH₂)ₙR₉; —C(═O)—R₁₀; —NR₁₁R₁₂; and a saturated or unsaturated, straight or branched, cyclic or acyclic, chiral or achiral hydrocarbyl group having from 1 to 10 carbon atoms, wherein the hydrocarbyl group is optionally substituted with one or more of hydroxy, (C₁-C₄)alkoxy, amino, (C₁-C₄)alkylamino, (C₁-C₄)dialkylamino, halogen, cyano, nitro, carboxy, (C₁-C₄)alkoxycarbonyl or sulfonamido;
R₂ is selected from the group consisting of: H; halogen; nitro; cyano; —O—R₈; —O—C(═O)—(CH₂)ₙR₉; —C(═O)—R₁₀; —NR₁₁R₁₂; and a saturated or unsaturated, straight or branched, cyclic or acyclic, chiral or achiral hydrocarbyl group having from 1 to 10 carbon atoms, wherein the hydrocarbyl group is optionally substituted with one or more of hydroxy, (C₁-C₄)alkoxy, amino, (C₁-C₄)alkylamino, (C₁-C₄)dialkylamino, halogen, cyano, nitro, carboxy, (C₁-C₄)alkoxycarbonyl or sulfonamido;
R₃ is selected from the group consisting of: H; halogen; nitro; cyano; —O—R₈; —O—C(═O)—(CH₂)ₙR₉; —C(═O)—R₁₀; —NR₁₁R₁₂; and a saturated or unsaturated, straight or branched, cyclic or acyclic, chiral or achiral hydrocarbyl group having from 1 to 10 carbon atoms, wherein the hydrocarbyl group is optionally substituted with one or more of hydroxy, (C₁-C₄)alkoxy, amino, (C₁-C₄)alkylamino, (C₁-C₄)dialkylamino, halogen, cyano, nitro, carboxy, (C₁-C₄)alkoxycarbonyl or sulfonamido;
R₄ is selected from the group consisting of: H; halogen; nitro; cyano; —O—R₈; —O—C(═O)—(CH₂)ₙR₉; —C(═O)—R₁₀; —NR₁₁R₁₂; and a saturated or unsaturated, straight or branched, cyclic or acyclic, chiral or achiral hydrocarbyl group having from 1 to 10 carbon atoms, wherein the hydrocarbyl group is optionally substituted with one or more of hydroxy, (C₁-C₄)alkoxy, amino, (C₁-C₄)alkylamino, (C₁-C₄)dialkylamino, halogen, cyano, nitro, carboxy, (C₁-C₄)alkoxycarbonyl or sulfonamido;
R₅ is selected from the group consisting of CN; NO₂; (C₁-C₆)perfluoroalkyl; substituted or unsubstituted (C₆-C₁₀)aryl; substituted or unsubstituted (C₂-C₉)heterocyclyl; substituted or unsubstituted (C₇-C₁₅)aralkyl; substituted or unsubstituted (C₂-C₉)heterocyclylalkyl; and —C(═O)—R₁₃;
R₆ is selected from the group consisting of H; a saturated or unsaturated, straight or branched, cyclic or acyclic, chiral or achiral hydrocarbyl group having from 1 to 10 carbon atoms, wherein the hydrocarbyl group is substituted with (C₁-C₆)carboxy, (C₁-C₆)carboxyalkyl, hydroxy, or (C₁-C₆) alkoxy; and substituted or unsubstituted (C₁₁-C₁₅)aralkyl;
R₇ is selected from the group consisting of H; saturated or unsaturated, straight or branched, cyclic or acyclic, chiral or achiral hydrocarbyl group having from 1 to 10 carbon atoms, wherein the hydrocarbyl group is substituted with (C₁-C₆)carboxy, (C₁-C₆)alkoxycarbonyl, hydroxy, or (C₁-C₆) alkoxy; substituted or unsubstituted (C₇-C₁₅)aralkyl; and —C(═O)—R₁₄;
or
R₆ and R₇ combine to form ═CH—NR₁₇R₁₈;
R₈ is independently selected from the group consisting of:

H;

a saturated or unsaturated, straight or branched, cyclic or acyclic, chiral or achiral hydrocarbyl group having from 1 to 10 carbon atoms, wherein the hydrocarbyl group is optionally substituted with one or more of halogen, hydroxy, (C₁-C₄)alkylcarbonyloxy, or —NR₁₁R₁₂, and wherein one or more of the carbon atoms in the hydrocarbyl group is optionally replaced by an oxygen atom (—O—);

substituted or unsubstituted (C₆-C₁₀)aryl;

substituted or unsubstituted (C₂-C₉)heterocyclyl; and

—NH₂;

R₉ is independently selected from the group consisting of (C₁-C₆)alkyl; substituted or unsubstituted (C₆-C₁₀)aryl; substituted or unsubstituted (C₇-C₁₅)aralkyl; substituted or unsubstituted (C₂-C₉)heterocyclyl; substituted or unsubstituted (C₂-C₉)heterocyclylalkyl; and —NH₂;
R₁₀ is independently selected from the group consisting of —OR₁₅; —NR₁₁R₁₂, and a saturated or unsaturated, straight or branched, cyclic or acyclic, chiral or achiral hydrocarbyl group having from 1 to 10 carbon atoms, wherein the hydrocarbyl group is optionally substituted with one or more of halogens, hydroxy, alkoxy, cyano, carboxy, carbalkoxy, carboxamide, —NR₁₁R₁₂, or nitro;
R₁₁ and R₁₂ are each independently selected from the group consisting of H; (C₂-C₄)acyl; saturated or unsaturated, straight or branched, cyclic or acyclic, chiral or achiral hydrocarbyl group having from 1 to 10 carbon atoms; and substituted or unsubstituted (C₂-C₉)heterocyclyl;

wherein R₁₁ and R₁₂ may combine with O, S, or the nitrogen atom in NR₁₆ to form a substituted or unsubstituted heterocyclyl ring;

R₁₃ is selected from the group consisting of —OR₁₅; and a saturated or unsaturated, straight or branched, cyclic or acyclic, chiral or achiral hydrocarbyl group having from 1 to 10 carbon atoms, wherein the hydrocarbyl group is substituted with one or more of halogens, hydroxy, alkoxy, cyano, carboxy, carbalkoxy, carboxamido, or nitro;
R₁₄ is selected from the group consisting of:

substituted or unsubstituted (C₂-C₉)heterocyclyl;

—C(═O)-substituted or unsubstituted (C₆-C₁₀)aryl;

—C(═O)-substituted or unsubstituted (C₂-C₉)heterocyclyl;

—C(═O)—NH-substituted or unsubstituted (C₆-C₁₀)aryl;

—C(═O)—NH-substituted or unsubstituted (C₆-C₁₀)arylkyl;

—C(═O)—NH-substituted or unsubstituted (C₂-C₉)heterocyclyl;

—C(═O)-substituted or unsubstituted (C₂-C₉)heterocyclyl-substituted or unsubstituted (C₆-C₁₀)heteroaryl; and

—C(═O)—NH-substituted or unsubstituted (C₆-C₁₀)aryl-substituted or unsubstituted (C₂-C₉)heterocyclyl.

R₁₅ is independently selected from the group consisting of (C₁-C₆)alkyl; substituted or unsubstituted (C₇-C₁₅)aralkyl; substituted or unsubstituted (C₂-C₉)heterocyclyl; and substituted or unsubstituted (C₂-C₉)heterocyclylalkyl;
R₁₆ is selected from the group consisting of H; substituted or unsubstituted (C₁-C₆)alkyl; substituted or unsubstituted (C₆-C₁₀)aryl; and substituted or unsubstituted (C₇-C₁₅)aralkyl;
R₁₇ and R₁₈ are each independently (C₁-C₆)alkyl;
n is independently selected from the group consisting of 0, 1, 2, 3, 4, and 5;
X is absent or selected from (C₁-C₆)alkyl and (C₁-C₆)alkoxyoxo(C₁-C₆)alkyl; and
Y is absent or a counterion, preferably a halide,
i) provided that when R₆ is H, R₇ is H, and R₁₃ is —OR₁₅, then at least one of R₂ and R₃ is —O—R₈, wherein R₈ is selected from the group consisting of: H; a saturated or unsaturated, straight or branched, cyclic or acyclic, chiral or achiral hydrocarbyl group having from 1 to 10 carbon atoms, wherein the hydrocarbyl group is substituted with one or more of halogen, hydroxy, (C₁-C₄)alkylcarbonyloxy, or —NR₁₁R₁₂; substituted or unsubstituted (C₆-C₁₀)aryl; substituted or unsubstituted (C₂-C₉)heterocyclyl; and —NH₂;
ii) provided that when X is absent, then the N⁺ of Formula I is N and Y is absent, and
iii) when X is (C₁-C₆)alkyl or (C₁-C₆)alkoxyoxo(C₁-C₆)alkyl, then Y is a counterion.

In certain embodiments, the salt of a compound of Formula I is a pharmaceutically acceptable salt.

Another particular embodiment of the invention comprises a compound of Formula I, or a salt thereof, wherein R₁ is H, and R₄ is H.

Another particular embodiment of the invention comprises a compound of Formula I, or a salt thereof, wherein R₁ is H, and R₄ is H; R₂ is —O—R₈; and R₈ is independently selected from the group consisting of H and a saturated or unsaturated, straight or branched, cyclic or acyclic, chiral or achiral hydrocarbyl group having from 1 to 10 carbon atoms.

Another particular embodiment of the invention comprises a compound of Formula I, or a salt thereof, wherein R₁ is H, and R₄ is H; R₃ is —O—R₈, and R₈ is independently selected from the group consisting of H and a saturated or unsaturated, straight or branched, cyclic or acyclic, chiral or achiral hydrocarbyl group having from 1 to 10 carbon atoms.

Another particular embodiment of the invention comprises a compound of Formula I, or a salt thereof, wherein R₁ is H, and R₄ is H; R₂ is —O—R₈, and R₈ is independently selected from the group consisting of H and substituted or unsubstituted (C₆-C₁₀)aryl.

Another particular embodiment of the invention comprises a compound of Formula I, or a salt thereof, wherein R₁ is H, and R₄ is H; R₃ is —O—R₈, and R₈ is independently selected from the group consisting of H and substituted or unsubstituted (C₆-C₁₀)aryl.

Another particular embodiment of the invention comprises a compound of Formula I, or a salt thereof, wherein R₆ is H, R₇ is H, R₁₃ is —OR₁₅, and at least one of R₂ and R₃ is —O—H.

Another particular embodiment of the invention comprises a compound of Formula I, or a salt thereof, wherein R₆ is H, R₇ is H, R₁₃ is —OR₁₅, and at least one of R₂ and R₃ is —O—R₈, wherein R₈ is a saturated or unsaturated, straight or branched, cyclic or acyclic, chiral or achiral hydrocarbyl group having from 1 to 10 carbon atoms, wherein the hydrocarbyl group is substituted with —NR₁₁R₁₂.

Another particular embodiment of the invention comprises a compound of Formula I, or a salt thereof, wherein R₆ is H, R₇ is H, R₁₃ is —OR₁₅, and at least one of R₂ and R₃ is —O—R₈, wherein R₈ is a saturated or unsaturated, straight or branched, cyclic or acyclic, chiral or achiral hydrocarbyl group having from 1 to 10 carbon atoms, wherein the hydrocarbyl group is substituted with one or more hydroxy group.

Another particular embodiment of the invention comprises a compound of Formula I, or a salt thereof, wherein R₆ is H, R₇ is H, R₁₃ is —OR₁₅, and at least one of R₂ and R₃ is —O—R₈, wherein R₈ is a saturated or unsaturated, straight or branched, cyclic or acyclic, chiral or achiral hydrocarbyl group having from 1 to 10 carbon atoms, wherein the hydrocarbyl group is substituted with one or more of halogen.

Another particular embodiment of the invention comprises a compound of Formula I, or a salt thereof, wherein R₆ is H, R₇ is H, R₁₃ is —OR₁₅, and at least one of R₂ and R₃ is —O—R₈, wherein R₈ is a saturated or unsaturated, straight or branched, cyclic or acyclic, chiral or achiral hydrocarbyl group having from 1 to 10 carbon atoms, wherein the hydrocarbyl group is substituted with one or more (C₁-C₄)alkylcarbonyloxy groups.

Another particular embodiment of the invention comprises a compound of Formula I, or a salt thereof, wherein R₆ is H, R₇ is H, R₁₃ is —OR₁₅, and at least one of R₂ and R₃ is —O—R₈, wherein R₈ is substituted or unsubstituted (C₆-C₁₀)aryl.

Another particular embodiment of the invention comprises a compound of Formula I, or a salt thereof, wherein R₆ is H, R₇ is H, R₁₃ is —OR₁₅, and at least one of R₂ and R₃ is —O—R₈, wherein R₈ is substituted or unsubstituted (C₂-C₉)heterocyclyl.

Another particular embodiment of the invention comprises a compound of Formula I, or a salt thereof, wherein R₆ is H, R₇ is H, R₁₃ is —OR₁₅, and at least one of R₂ and R₃ is —O—NH₂.

Another particular embodiment of the invention comprises a compound of Formula I, or a salt thereof, wherein R₁ is H, and R₄ is H; R₃ is —O—C(═O)—R₉; and R₉ is substituted or unsubstituted (C₂-C₉)heterocyclyl.

Another particular embodiment of the invention comprises a compound of Formula I, or a salt thereof, wherein R₅ is substituted or unsubstituted (C₂-C₉)heterocyclyl.

Another particular embodiment of the invention comprises a compound of Formula I, or a salt thereof, wherein R₅ is CN.

Another particular embodiment of the invention comprises a compound of Formula I, or a salt thereof, wherein R₅ is —C(═O)—R₁₃; and R₁₃ is a saturated or unsaturated, straight or branched, cyclic or acyclic, chiral or achiral hydrocarbyl group having from 1 to 10 carbon atoms, wherein the hydrocarbyl group is substituted with one or more of halogen.

Another particular embodiment of the invention comprises a compound of Formula I, or a salt thereof, wherein R₆ is H.

Another particular embodiment of the invention comprises a compound of Formula I, or a salt thereof, wherein R₆ is a saturated or unsaturated, straight or branched, cyclic or acyclic, chiral or achiral hydrocarbyl group having from 1 to 10 carbon atoms, wherein the hydrocarbyl group is substituted with (C₁-C₆)carboxy, (C₁-C₆)carboxyalkyl, hydroxy, or (C₁-C₆) alkoxy.

Another particular embodiment of the invention comprises a compound of Formula I, or a salt thereof, wherein R₆ is substituted or unsubstituted (C₁₁-C₁₅)aralkyl.

Another particular embodiment of the invention comprises a compound of Formula I, or a salt thereof, wherein R₇ is H.

Another particular embodiment of the invention comprises a compound of Formula I, or a salt thereof, wherein R₆ is H; and R₇ is H.

Another particular embodiment of the invention comprises a compound of Formula I, or a salt thereof, wherein R₇ is —C(═O)—R₁₄; and R₁₄ is substituted or unsubstituted (C₂-C₉)heterocyclyl.

Another particular embodiment of the invention comprises a compound of Formula I, or a salt thereof, wherein R₇ is —C(═O)—R₁₄; and R₁₄ is —C(═O)-substituted or unsubstituted (C₆-C₁₀)aryl.

Another particular embodiment of the invention comprises a compound of Formula I, or a salt thereof, wherein R₇ is —C(═O)—R₁₄; and R₁₄ is —C(═O)-substituted or unsubstituted (C₂-C₉)heterocyclyl.

Another particular embodiment of the invention comprises a compound of Formula I, or a salt thereof, wherein R₇ is —C(═O)—R₁₄; and R₁₄ is —C(═O)—NH-substituted or unsubstituted (C₆-C₁₀)aryl.

Another particular embodiment of the invention comprises a compound of Formula I, or a salt thereof, wherein R₇ is —C(═O)—R₁₄; and R₁₄ is —C(═O)—NH-substituted or unsubstituted (C₆-C₁₀)arylkyl.

Another particular embodiment of the invention comprises a compound of Formula I, or a salt thereof, wherein R₇ is —C(═O)—R₁₄; and R₁₄ is —C(═O)—NH-substituted or unsubstituted (C₂-C₉)heterocyclyl.

Another particular embodiment of the invention comprises a compound of Formula I, or a salt thereof, wherein R₇ is —C(═O)—R₁₄; and R₁₄ is —C(═O)-substituted or unsubstituted (C₂-C₉)heterocyclyl-substituted or unsubstituted (C₆-C₁₀)heteroaryl.

Another particular embodiment of the invention comprises a compound of Formula I, or a salt thereof, wherein R₇ is —C(═O)—R₁₄; and R₁₄ is —C(═O)—NH-substituted or unsubstituted (C₆-C₁₀)aryl-substituted or unsubstituted (C₂-C₉)heterocyclyl.

In another aspect, the invention is a compound of Formula I, or a salt thereof wherein:

R₁ is selected from the group consisting of: H; halogen; nitro; cyano; —O—R₈; —O—C(═O)—(CH₂)ₙR₉; —C(═O)—R₁₀; —NR₁₁R₁₂; and a saturated or unsaturated, straight or branched, cyclic or acyclic, chiral or achiral hydrocarbyl group having from 1 to 10 carbon atoms, wherein the hydrocarbyl group is optionally substituted with one or more of hydroxy, (C₁-C₄)alkoxy, amino, (C₁-C₄)alkylamino, (C₁-C₄)dialkylamino, halogen, cyano, nitro, carboxy, (C₁-C₄)alkoxycarbonyl or sulfonamido;
R₂ is selected from the group consisting of: H; halogen; nitro; cyano; —O—R₈; —O—C(═O)—(CH₂)ₙR₉; —C(═O)—R₁₀; —NR₁₁R₁₂; and a saturated or unsaturated, straight or branched, cyclic or acyclic, chiral or achiral hydrocarbyl group having from 1 to 10 carbon atoms, wherein the hydrocarbyl group is optionally substituted with one or more of hydroxy, (C₁-C₄)alkoxy, amino, (C₁-C₄)alkylamino, (C₁-C₄)dialkylamino, halogen, cyano, nitro, carboxy, (C₁-C₄)alkoxycarbonyl or sulfonamido;
R₃ is selected from the group consisting of: H; halogen; nitro; cyano; —O—R₈; —O—C(═O)—(CH₂)ₙR₉; —C(═O)—R₁₀; —NR₁₁R₁₂; and a saturated or unsaturated, straight or branched, cyclic or acyclic, chiral or achiral hydrocarbyl group having from 1 to 10 carbon atoms, wherein the hydrocarbyl group is optionally substituted with one or more of hydroxy, (C₁-C₄)alkoxy, amino, (C₁-C₄)alkylamino, (C₁-C₄)dialkylamino, halogen, cyano, nitro, carboxy, (C₁-C₄)alkoxycarbonyl or sulfonamido;
R₄ is selected from the group consisting of: H; halogen; nitro; cyano; —O—R₈; —O—C(═O)—(CH₂)ₙR₉; —C(═O)—R₁₀; —NR₁₁R₁₂; and a saturated or unsaturated, straight or branched, cyclic or acyclic, chiral or achiral hydrocarbyl group having from 1 to 10 carbon atoms, wherein the hydrocarbyl group is optionally substituted with one or more of hydroxy, (C₁-C₄)alkoxy, amino, (C₁-C₄)alkylamino, (C₁-C₄)dialkylamino, halogen, cyano, nitro, carboxy, (C₁-C₄)alkoxycarbonyl or sulfonamido;
R₅ is selected from the group consisting of CN; NO₂; (C₁-C₆)perfluoroalkyl; substituted or unsubstituted (C₆-C₁₀)aryl; substituted or unsubstituted (C₂-C₉)heterocyclyl; substituted or unsubstituted (C₇-C₁₅)aralkyl; substituted or unsubstituted (C₂-C₉)heterocyclylalkyl; and —C(═O)—R₁₃;

### R₆ is H;

R₇ is selected from the group consisting of H; and —C(═O)—R₁₄;
or
R₆ and R₇ combine to form ═CH—NR₁₇R₁₈;
R₈ is independently selected from the group consisting of:

H;

a saturated or unsaturated, straight or branched, cyclic or acyclic, chiral or achiral hydrocarbyl group having from 1 to 10 carbon atoms, wherein the hydrocarbyl group is optionally substituted with one or more of halogen, hydroxy, (C₁-C₄)alkylcarbonyloxy, or —NR₁₁R₁₂, and wherein one or more of the carbon atoms in the hydrocarbyl group is optionally replaced by an oxygen atom (—O—);

substituted or unsubstituted (C₆-C₁₀)aryl;

substituted or unsubstituted (C₂-C₉)heterocyclyl; and

—NH₂;

R₉ is independently selected from the group consisting of (C₁-C₆)alkyl; substituted or unsubstituted (C₆-C₁₀)aryl; substituted or unsubstituted (C₇-C₁₅)aralkyl; substituted or unsubstituted (C₂-C₉)heterocyclyl; substituted or unsubstituted (C₂-C₉)heterocyclylalkyl; and —NH₂;
R₁₀ is independently selected from the group consisting of —OR_{B}; —NR₁₁R₁₂, and a saturated or unsaturated, straight or branched, cyclic or acyclic, chiral or achiral hydrocarbyl group having from 1 to 10 carbon atoms, wherein the hydrocarbyl group may be optionally substituted with one or more of halogens, hydroxy, alkoxy, cyano, carboxy, carbalkoxy, carboxamide, —NR₁₁R₁₂, or nitro;
R₁₁ and R₁₂ are each independently selected from the group consisting of H; (C₂-C₄)acyl; saturated or unsaturated, straight or branched, cyclic or acyclic, chiral or achiral hydrocarbyl group having from 1 to 10 carbon atoms; and substituted or unsubstituted (C₂-C₉)heterocyclyl;

wherein R₁₁ and R₁₂ may combine with O, S, or the nitrogen atom in NR₁₆ to form a substituted or unsubstituted heterocyclyl ring;

R₁₃ is selected from the group consisting of —OR_{B}; and (C₁-C₆)perfluoroalkyl;
R₁₄ is selected from the group consisting of:

substituted or unsubstituted (C₂-C₉)heterocyclyl;

—C(═O)-substituted or unsubstituted (C₆-C₁₀)aryl;

—C(═O)-substituted or unsubstituted (C₂-C₉)heterocyclyl;

—C(═O)—NH-substituted or unsubstituted (C₆-C₁₀)aryl;

—C(═O)—NH-substituted or unsubstituted (C₆-C₁₀)arylkyl;

—C(═O)—NH-substituted or unsubstituted (C₂-C₉)heterocyclyl;

—C(═O)-substituted or unsubstituted (C₂-C₉)heterocyclyl-substituted or unsubstituted (C₆-C₁₀)heteroaryl; and

—C(═O)—NH-substituted or unsubstituted (C₆-C₁₀)aryl-substituted or unsubstituted (C₂-C₉)heterocyclyl;

R₁₅ is (C₁-C₆)alkyl;
R₁₆ is selected from the group consisting of H; substituted or unsubstituted (C₁-C₆)alkyl; substituted or unsubstituted (C₆-C₁₀)aryl; and substituted or unsubstituted (C₇-C₁₅)aralkyl;
R₁₇ and R₁₈ are each independently (C₁-C₆)alkyl;
n is independently selected from the group consisting of 0, 1, 2, 3, 4, and 5;
X is absent or selected from (C₁-C₆)alkyl and (C₁-C₆)alkoxyoxo(C₁-C₆)alkyl; and
Y is absent or a counterion, preferably halide,
i) provided that when R₇ is H, and R₁₃ is —OR₁₅, then at least one of R₂ and R₃ is —O—R₈, wherein R₈ is selected from the group consisting of: H, —(CH₂)₂-heterocyclyl, and —(CH₂)₃-heterocyclyl;
ii) provided that when X is absent, then the N⁺ of Formula I is N and Y is absent, and
iii) when X is an (C₁-C₆)alkyl or (C₁-C₆)alkoxyoxo(C₁-C₆)alkyl, then Y is a counterion.

Another particular embodiment of the invention comprises a compound of Formula I, or a salt thereof, wherein R₆ is H, R₇ is selected from the group consisting of H; and —C(═O)—R₁₄, R₁₃ is —OR₁₅, and at least one of R₂ and R₃ is —O—H.

Another particular embodiment of the invention comprises a compound of Formula I, or a salt thereof, wherein R₆ is H, R₇ is selected from the group consisting of H; and —C(═O)—R₁₄, R₁₃ is —OR₁₅, and at least one of R₂ and R₃ is —O—(CH₂)₂-heterocyclyl.

Another particular embodiment of the invention comprises a compound of Formula I, or a salt thereof, wherein R₆ is H, R₇ is selected from the group consisting of H; and —C(═O)—R₁₄, R₁₃ is —OR₁₅, and at least one of R₂ and R₃ is —O—(CH₂)₃-heterocyclyl.

Another particular embodiment of the invention comprises a compound of Formula I, or a salt thereof, wherein R₆ is H, and R₇ is —C(═O)—R₁₄.

In another aspect, the invention is a compound of Formula I, or a salt thereof wherein:

R₁ is selected from the group consisting of: H; halogen; nitro; cyano; —O—R₈; —O—C(═O)—(CH₂)ₙR₉; —C(═O)—R₁₀; —NR₁₁R₁₂; and a saturated or unsaturated, straight or branched, cyclic or acyclic, chiral or achiral hydrocarbyl group having from 1 to 10 carbon atoms, wherein the hydrocarbyl group is optionally substituted with one or more of hydroxy, (C₁-C₄)alkoxy, amino, (C₁-C₄)alkylamino, (C₁-C₄)dialkylamino, halogen, cyano, nitro, carboxy, (C₁-C₄)alkoxycarbonyl or sulfonamido;
R₂ is selected from the group consisting of: H; halogen; nitro; cyano; —O—R₈; —O—C(═O)—(CH₂)ₙR₉; —C(═O)—R₁₀; —NR₁₁R₁₂; and a saturated or unsaturated, straight or branched, cyclic or acyclic, chiral or achiral hydrocarbyl group having from 1 to 10 carbon atoms, wherein the hydrocarbyl group is optionally substituted with one or more of hydroxy, (C₁-C₄)alkoxy, amino, (C₁-C₄)alkylamino, (C₁-C₄)dialkylamino, halogen, cyano, nitro, carboxy, (C₁-C₄)alkoxycarbonyl or sulfonamido;
R₃ is selected from the group consisting of: H; halogen; nitro; cyano; —O—R₈; —O—C(═O)—(CH₂)ₙR₉; —C(═O)—R₁₀; —NR₁₁R₁₂; and a saturated or unsaturated, straight or branched, cyclic or acyclic, chiral or achiral hydrocarbyl group having from 1 to 10 carbon atoms, wherein the hydrocarbyl group is optionally substituted with one or more of hydroxy, (C₁-C₄)alkoxy, amino, (C₁-C₄)alkylamino, (C₁-C₄)dialkylamino, halogen, cyano, nitro, carboxy, (C₁-C₄)alkoxycarbonyl or sulfonamido;
R₄ is selected from the group consisting of: H; halogen; nitro; cyano; —O—R₈; —O—C(═O)—(CH₂)ₙR₉; —C(═O)—R₁₀; —NR₁₁R₁₂; and a saturated or unsaturated, straight or branched, cyclic or acyclic, chiral or achiral hydrocarbyl group having from 1 to 10 carbon atoms, wherein the hydrocarbyl group is optionally substituted with one or more of hydroxy, (C₁-C₄)alkoxy, amino, (C₁-C₄)alkylamino, (C₁-C₄)dialkylamino, halogen, cyano, nitro, carboxy, (C₁-C₄)alkoxycarbonyl or sulfonamido;
R₅ is selected from the group consisting of CN; NO₂; (C₁-C₆)perfluoroalkyl; substituted or unsubstituted (C₆-C₁₀)aryl; substituted or unsubstituted (C₂-C₉)heterocyclyl; substituted or unsubstituted (C₇-C₁₅)aralkyl; substituted or unsubstituted (C₂-C₉)heterocyclylalkyl; and —C(═O)—R₁₃;

### R₆ is H;

### R₇ is —C(═O)—R₁₄;

or
R₆ and R₇ combine to form ═CH—NR₁₇R₁₈;
R₈ is independently selected from the group consisting of:

H;

a saturated or unsaturated, straight or branched, cyclic or acyclic, chiral or achiral hydrocarbyl group having from 1 to 10 carbon atoms, wherein the hydrocarbyl group is optionally substituted with one or more of halogen, hydroxy, (C₁-C₄)alkylcarbonyloxy, or —NR₁₁R₁₂, and wherein one or more of the carbon atoms in the hydrocarbyl group is optionally replaced by an oxygen atom (—O—);

substituted or unsubstituted (C₆-C₁₀)aryl;

substituted or unsubstituted (C₂-C₉)heterocyclyl; and

—NH₂;

R₉ is independently selected from the group consisting of (C₁-C₆)alkyl; substituted or unsubstituted (C₆-C₁₀)aryl; substituted or unsubstituted (C₇-C₁₅)aralkyl; substituted or unsubstituted (C₂-C₉)heterocyclyl; substituted or unsubstituted (C₂-C₉)heterocyclylalkyl; and —NH₂;
R₁₀ is independently selected from the group consisting of —OR₁₅; —NR₁₁R₁₂; and a saturated or unsaturated, straight or branched, cyclic or acyclic, chiral or achiral hydrocarbyl group having from 1 to 10 carbon atoms, wherein the hydrocarbyl group may be optionally substituted with one or more of halogens, hydroxy, alkoxy, cyano, carboxy, carbalkoxy, carboxamido, —NR₁₁R₁₂, or nitro;
R₁₁ and R₁₂ are each independently selected from the group consisting of H; (C₂-C₄)acyl; saturated or unsaturated, straight or branched, cyclic or acyclic, chiral or achiral hydrocarbyl group having from 1 to 10 carbon atoms; and substituted or unsubstituted (C₂-C₉)heterocyclyl;

wherein R₁₁ and R₁₂ may combine with O, S, or the nitrogen atom in NR₁₆ to form a substituted or unsubstituted heterocyclyl ring;

R₁₃ is selected from the group consisting of —OR₁₅; —NR₁₁R₁₂; and a saturated or unsaturated, straight or branched, cyclic or acyclic, chiral or achiral hydrocarbyl group having from 1 to 10 carbon atoms, wherein the hydrocarbyl group may be optionally substituted with one or more of halogens, hydroxy, alkoxy, cyano, carboxy, carbalkoxy, carboxamido, —NR₁₁R₁₂ and nitro;

R₁₄ is selected from the group consisting of:

substituted or unsubstituted (C₂-C₉)heterocyclyl;

—C(═O)-substituted or unsubstituted (C₆-C₁₀)aryl;

—C(═O)-substituted or unsubstituted (C₂-C₉)heterocyclyl;

—C(═O)—NH-substituted or unsubstituted (C₆-C₁₀)aryl;

—C(═O)—NH-substituted or unsubstituted (C₆-C₁₀)arylkyl;

—C(═O)—NH-substituted or unsubstituted (C₂-C₉)heterocyclyl;

—C(═O)-substituted or unsubstituted (C₂-C₉)heterocyclyl-substituted or unsubstituted (C₆-C₁₀)heteroaryl; and

—C(═O)—NH-substituted or unsubstituted (C₆-C₁₀)aryl-substituted or unsubstituted (C₂-C₉)heterocyclyl.

R₁₅ is independently selected from the group consisting of H; (C₁-C₆)alkyl; substituted or unsubstituted (C₆-C₁₀)aryl; substituted or unsubstituted (C₉-C₁₅)aralkyl; substituted or unsubstituted (C₂-C₉)heterocyclyl; and substituted or unsubstituted (C₂-C₉)heterocyclylalkyl;
R₁₆ is selected from the group consisting of H; substituted or unsubstituted (C₁-C₆)alkyl; substituted or unsubstituted (C₆-C₁₀)aryl; and substituted or unsubstituted (C₇-C₁₅)aralkyl;
R₁₇ and R₁₈ are each independently (C₁-C₆)alkyl;
n is independently selected from the group consisting of 0, 1, 2, 3, 4, and 5;
X is absent or selected from (C₁-C₆)alkyl and (C₁-C₆)alkoxyoxo(C₁-C₆)alkyl; and
Y is absent or a counterion, preferably halide,
i) provided that when X is absent, then the N⁺ of Formula I is N and Y is absent, and
ii) when X is an (C₁-C₆)alkyl or (C₁-C₆)alkoxyoxo(C₁-C₆)alkyl, then Y is a counterion.

In another aspect, the invention is a compound of Formula I, or a salt thereof wherein:

### R₁ is H;

### R₂ is —O—R₈;

R₃ is selected from the group consisting of —O—R₈; and —O—C(═O)—(CH₂)ₙR₉;

### R₄ is H;

R₅ is selected from the group consisting of CN; substituted or unsubstituted (C₂-C₉)heterocyclyl; and —C(═O)—R₁₃;

### R₆ is H;

R₇ is selected from the group consisting of H; —C(═O)—R₁₄; or R₆ and R₇ form —N═CH—NR₁₇R₁₈;
R₈ is independently selected from the group consisting of a saturated or unsaturated, straight or branched, cyclic or acyclic, chiral or achiral hydrocarbyl group having from 1 to 10 carbon atoms, wherein the hydrocarbyl group is optionally substituted with one or more of halogen, hydroxy, (C₁-C₄)alkylcarbonyloxy, or —NR₁₁R₁₂, and wherein one or more of the carbon atoms in the hydrocarbyl group is optionally replaced by an oxygen atom (—O—);
R₉ is substituted or unsubstituted (C₂-C₉)heterocyclyl;
R₁₀ is independently selected from the group consisting of —OR₁₅; —NR₁₁R₁₂; and a saturated or unsaturated, straight or branched, cyclic or acyclic, chiral or achiral hydrocarbyl group having from 1 to 10 carbon atoms, wherein the hydrocarbyl group may be optionally substituted with one or more of halogens, hydroxy, alkoxy, cyano, carboxy, carbalkoxy, carboxamide, —NR₁₁R₁₂, or nitro;
R₁₁ and R₁₂ are H, or R₁₁ and R₁₂ may combine with O, S, or the nitrogen atom in NR₁₆ to form a substituted or unsubstituted heterocyclyl ring;
R₁₃ is selected from the group consisting of —OR_{B}; and a saturated or unsaturated, straight or branched, cyclic or acyclic, chiral or achiral hydrocarbyl group having from 1 to 10 carbon atoms, wherein the hydrocarbyl group is substituted with one or more halogens;
R₁₄ is selected from the group consisting of:

substituted or unsubstituted (C₂-C₉)heterocyclyl;

—C(═O)-substituted or unsubstituted (C₆-C₁₀)aryl;

—C(═O)-substituted or unsubstituted (C₂-C₉)heterocyclyl;

—C(═O)—NH-substituted or unsubstituted (C₆-C₁₀)aryl;

—C(═O)—NH-substituted or unsubstituted (C₆-C₁₀)arylkyl;

—C(═O)—NH-substituted or unsubstituted (C₂-C₉)heterocyclyl;

—C(═O)-substituted or unsubstituted (C₂-C₉)heterocyclyl-substituted or unsubstituted (C₆-C₁₀)heteroaryl; and

—C(═O)—NH-substituted or unsubstituted (C₆-C₁₀)aryl-substituted or unsubstituted (C₂-C₉)heterocyclyl;

R₁₅ is (C₁-C₆)alkyl;
R₁₆ is selected from the group consisting of H; and substituted or unsubstituted (C₁-C₆)alkyl;
R₁₇ and R₁₈ are each independently (C₁-C₆)alkyl;
n is 0;
X is absent or selected from (C₁-C₆)alkyl and (C₁-C₆)alkoxyoxo(C₁-C₆)alkyl; and
Y is absent or a counterion, preferably halide,
i) provided that when R₆ is H, R₇ is H, and R₁₃ is —OR₁₅, then at least one of R₂ and R₃ is —O—R₈, wherein R₈ is selected from the group consisting of: H; and a saturated or unsaturated, straight or branched, cyclic or acyclic, chiral or achiral hydrocarbyl group having from 1 to 10 carbon atoms, wherein the hydrocarbyl group is substituted with one or more —NR₁₁R₁₂;
ii) provided that when X is absent, then the N⁺ of Formula I is N and Y is absent, and
iii) when X is an (C₁-C₆)alkyl or (C₁-C₆)alkoxyoxo(C₁-C₆)alkyl, then Y is a counterion.

In another aspect, the invention is a compound of Formula I, or a salt thereof wherein:

at least one of R₁, R₂, R₃, and R₄ is selected from the group consisting of: H; a saturated or unsaturated, straight or branched, cyclic or acyclic, chiral or achiral hydrocarbyl group having from 1 to 10 carbon atoms, wherein the hydrocarbyl group is optionally substituted with one or more of halogen; and —O—R₈.

In another aspect, the invention is a compound of Formula I, or a salt thereof wherein:

at least one of R₁, R₂, R₃, and R₄ is H.

In another aspect, the invention is a compound of Formula I, or a salt thereof wherein: at least one of R₁, R₂, R₃, and R₄ is —O—R₈.

In another aspect, the invention is a compound of Formula I, or a salt thereof wherein:

R₅ is selected from the group consisting of H; CN; —C(═O)—R₁₃; substituted or unsubstituted (C₆-C₁₀)aryl; substituted or unsubstituted (C₇-C₁₅)aralkyl; substituted or unsubstituted (C₂-C₉)heterocyclylalkyl; and substituted or unsubstituted heterocyclyl.

In another aspect, the invention is a compound of Formula I, or a salt thereof wherein: R₆ is H.

In another aspect, the invention is a compound of Formula I, or a salt thereof wherein:

R₇ is selected from the group consisting of H; and —C(═O)—R₁₄; or R₆ and R₇ combine to form ═CH—NR₁₇R₁₈.

In another aspect, the invention is a compound of Formula I, or a salt thereof wherein:

### R₇ is —C(═O)—R₁₄, and

R₁₄ is selected from the group consisting of:

—C(═O)-substituted or unsubstituted (C₂-C₉)heterocyclyl;

—C(═O)-substituted or unsubstituted (C₂-C₉)heterocyclyl-substituted or unsubstituted (C₆-C₁₀)heteroaryl; and

—C(═O)—NH-substituted or unsubstituted (C₆-C₁₀)aryl-substituted or unsubstituted (C₂-C₉)heterocyclyl.

In another aspect, the invention is a compound of Formula I, or a salt thereof wherein: n is 0.

In certain embodiments, a compound of Formula I, or a salt thereof, is selected from the group consisting of:
(1) (3-Amino-7,8-dimethoxythieno[3,2-c]quinolin-2-yl)(4-fluorophenyl)methanone;
(2) Methyl 3-amino-7-benzyloxy-8-methoxythieno[3,2-c]quinolone-2-carboxylate;
(3) 1-(3-Amino-7,8-dimethoxythieno[3,2-c]quinolin-2-yl)-2,2,2-trifluoroethanone;
(4) Methyl 3-amino-8-benzyloxy-7-methoxythieno[3,2-c]quinoline-2-carboxylate;
(5) 3-Amino-7,8-dimethoxythieno[3,2-c]quinoline-2-carbonitrile;
(6) Methyl 3-amino-7-hydroxy-8-methoxythieno[3,2-c]quinoline-2-carboxylate;
(7) Methyl 3-amino-8-hydroxy-7-methoxythieno[3,2-c]quinoline-2-carboxylate;
(8) Methyl 3-amino-8-methoxy-7-(3-morpholinopropoxy)thieno[3,2-c]quinoline-2-carboxylate;
(9) Methyl 3-amino-8-methoxy-7-[3-(piperidin-1-yl)propoxy]thieno[3,2-c]quinoline-2-carboxylate;
(10) Methyl 3-amino-8-methoxy-7-[(4-methylpiperazine-1-carbonyl)oxy]thieno[3,2-c]quinoline-2-carboxylate;
(11) Methyl 3-amino-8-methoxy-7-(2-morpholinoethoxy)thieno[3,2-c]quinoline-2-carboxylate;
(12) Methyl 3-amino-7-methoxy-8-(3-morpholinopropoxy)thieno[3,2-c]quinoline-2-carboxylate;
(13) Methyl 7,8-dimethoxy-3-(4-methoxybenzamido)thieno[3,2-c]quinoline-2-carboxylate;
(14) Methyl 3-(2-fluorobenzamido)-7,8-dimethoxythieno[3,2-c]quinoline-2-carboxylate;
(15) Methyl 3-(4-fluorobenzamido)-7,8-dimethoxythieno[3,2-c]quinoline-2-carboxylate;
(16) Methyl 3-benzamido-7,8-dimethoxythieno[3,2-c]quinoline-2-carboxylate;
(17) Methyl 7,8-dimethoxy-3-(2-nitrobenzamido)thieno[3,2-c]quinoline-2-carboxylate;
(18) Methyl 7,8-dimethoxy-3-(2-aminobenzamido)thieno[3,2-c]quinoline-2-carboxylate;
(19) Methyl 3-(4-chloro-3-nitrobenzamido)-7,8-dimethoxythieno[3,2-c]quinoline-2-carboxylate;
(20) 7,8-dimethoxy-3-(2,2,2-trifluoroacetamido)thieno[3,2-c]quinoline-2-carboxylate;
(21) Methyl 3-(2-chloro-2-oxoacetamido)-7,8-dimethoxythieno[3,2-c]quinoline-2-carboxylate;
(22) Methyl 7,8-dimethoxy-3-[2-(4-methylpiperazin-1-yl)-2-oxoacetamido]thieno[3,2-c]quinoline-2-carboxylate;
(23) Methyl 7,8-dimethoxy-3-(2-morpholino-2-oxoacetamido)thieno[3,2-c]quinoline-2-carboxylate;
(24) Methyl 7,8-dimethoxy-3-{2-oxo-2-[4-(pyridin-4-yl)piperazin-1-yl]acetamido}thieno[3,2-c]quinoline-2-carboxylate;
(25) Methyl 7,8-dimethoxy-3-(2-{[4-(4-methylpiperazin-1-yl)phenyl]amino}-2-oxoacetamido)thieno[3,2-c]quinoline-2-carboxylate;
(26) Methyl 3-(2-chloroacetamido)-7,8-dimethoxythieno[3,2-c]quinoline-2-carboxylate;
(27) 3-amino-7,8-dimethoxy-2-(methoxycarbonyl)-5-methylthieno[3,2-c]quinolin-5-ium iodide;
(28) 3-amino-7,8-dimethoxy-5-(2-methoxy-2-oxoethyl)-2-(methoxycarbonyl)thieno[3,2-c]quinolin-5-ium bromide;
(29) (E)-Methyl 3-{[(dimethylamino)methylene]amino}-7,8-dimethoxythieno[3,2-c]quinoline-2-carboxylate;
(30) 7,8-Dimethoxy-2-(1H-tetrazol-5-yl)thieno[3,2-c]quinolin-3-amine; and
(31) 3-Amino-8-fluorothieno[3,2-c]quinoline-2-carbonitrile;
(32) (3-Amino-8-fluorothieno[3,2-c]quinolin-2-yl)(4-fluorophenyl)methanone;
(33) 3-Amino-7,8-difluorothieno[3,2-c]quinoline-2-carbonitrile;
(34) (3-Amino-7,8-difluorothieno[3,2-c]quinolin-2-yl)(4-fluorophenyl)methanone;
(35) (3-Amino-7,8-difluorothieno[3,2-c]quinolin-2-yl)(4-methoxyphenyl)methanone; and
(36) (3-Amino-8-fluorothieno[3,2-c]quinolin-2-yl)(4-methoxyphenyl)methanone.

### III. METHODS FOR PREPARING COMPOUNDS OF THE INVENTION AND INTERMEDIATES USEFUL IN THE SYNTHESIS OF COMPOUNDS OF THE INVENTION

There are provided processes for preparing compounds according to Formula I or Formula II, intermediates that are useful in the preparation of such compounds, and processes for preparing such intermediates.

Processes for producing embodiments of Formula I are disclosed, wherein the embodiments of Formula I are identified as Formula Ia, Formula Ib, Formula Ic, Formula Id, Formula Ie, Formula If, Formula Ig, Formula Ih, Formula Ii, Formula Ij, and Formula Ik. In the formulas and schemes that follow, unless otherwise indicated, R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, n, X, and Y are as defined above for Formula I.

A process for preparing a compound of Formula I is provided. The process comprises:

reacting a compound of Formula AA with a compound of Formula BB to produce a compound of Formula Ia

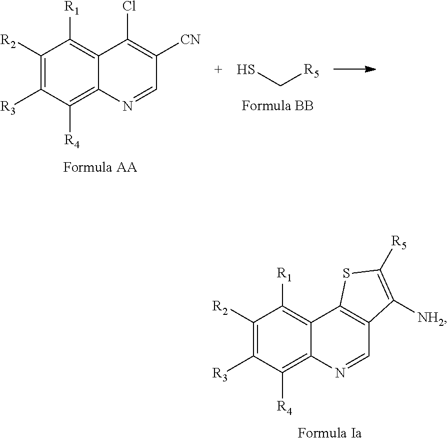

in a reaction mixture, wherein R₁, R₂, R₃, R₄, and R₅ are as defined above; and optionally, isolating from the reaction mixture the compound of Formula Ia or a salt thereof.

In an embodiment, the aforesaid reaction takes place in the presence of an acid or a base.

A process for preparing a compound of Formula I is provided. The process comprises:

reacting a compound of Formula CC with a compound of Formula DD to form a compound Formula Ib

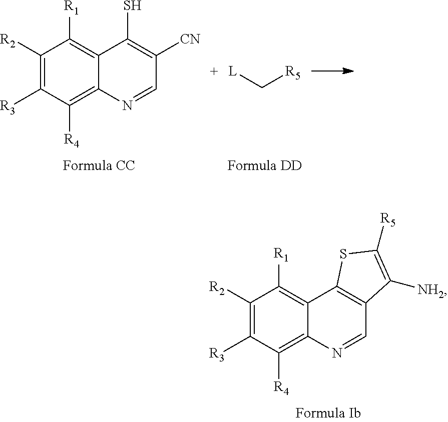

in a reaction mixture, wherein R₁, R₂, R₃, R₄, and R₅ are as defined above; and optionally, isolating from the reaction mixture the compound of Formula Ib or a salt thereof.

In an embodiment, the aforesaid reaction takes place in the presence of a base.

A process for preparing a compound of Formula I according to claim 1 is provided. The process comprises a reaction step selected from the group consisting of reaction steps (a), (b), and (c):

(a) reacting a compound of Formula HH with a compound of Formula IIa in the presence of a base to form a compound Formula Ie

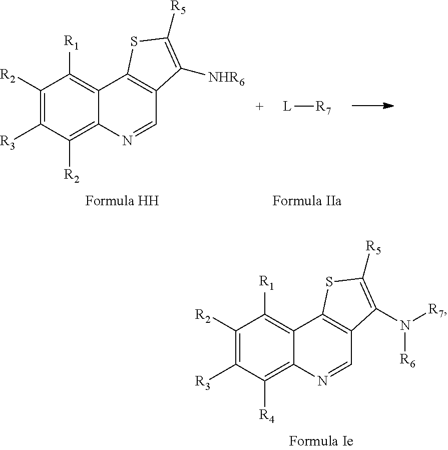

wherein R₁, R₂, R₃, R₄, R₅, R₆, and R₇ are as defined above and L is a halogen;

(b) reacting a compound of Formula JJ with a compound of Formula KK to form a compound Formula If

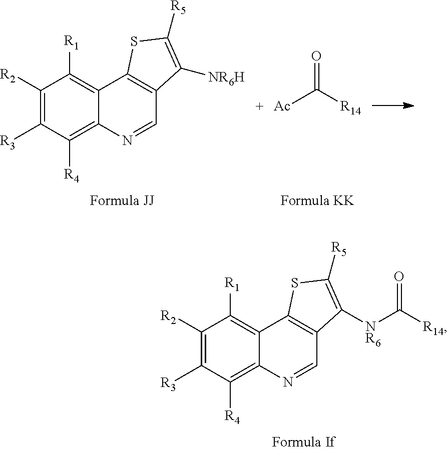

wherein R₁, R₂, R₃, R₄, R₅, R₆, and R₁₄ are as defined above, and Ac is an acyl group; and

(c) reacting a compound of Formula LL with a compound of Formula MM to form a compound Formula Ig

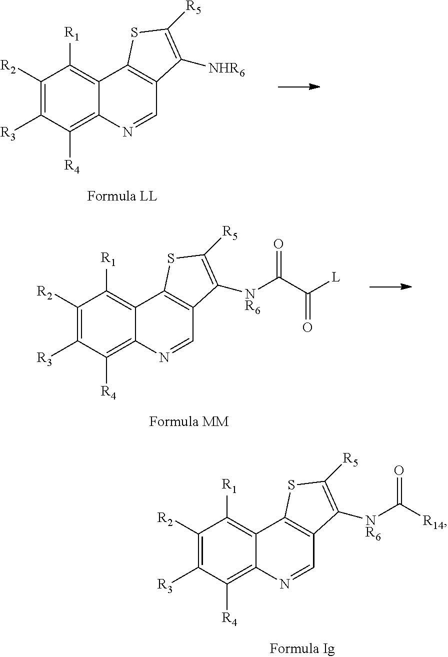

wherein R₁, R₂, R₃, R₄, R₅, R₆, and R₁₄ are as defined above and L is a halogen.

In an another embodiment, a process for preparing a compound of Formula I is provided. The process comprises:

condensing a compound of Formula PP with a compound XY to form a compound for Formula Ii

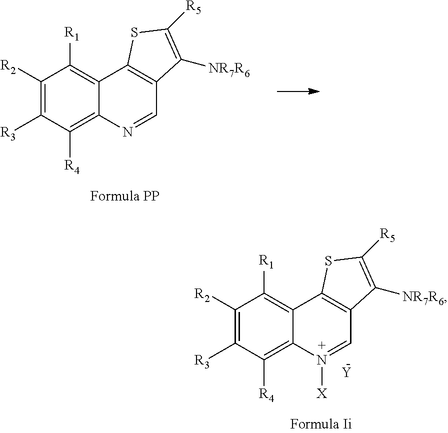

wherein R₁, R₂, R₃, R₄, R₅, R₆, R₇, X and Y are as defined above.

In the following paragraphs, a more detailed discussion of the processes for producing embodiments of Formula I is provided, along with preferred embodiments and reaction details. The embodiments of Formula I are identified as Formula Ia, Formula Ib, Formula Ic, Formula Id, Formula Ie, Formula If, Formula Ig, Formula Ih, Formula Ii, Formula Ij, and Formula Ik.

A process is provided in Scheme Ia for the synthesis of compounds according to Formula I comprising reacting a compound of Formula AA with a compound of Formula BB to produce a compound of Formula Ia, wherein Formula Ia is an embodiment of Formula I.

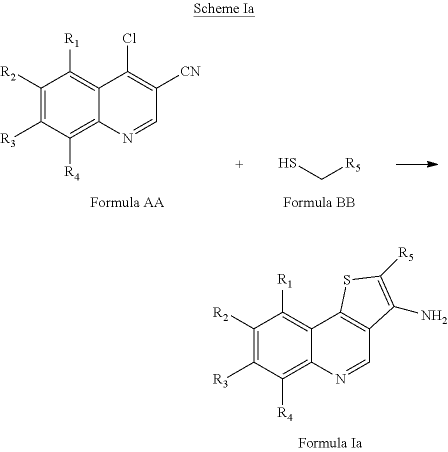

R₁, R₂, R₃, R₄, and R₅ are as defined above.

Particular embodiments of the process in Scheme Ia include those wherein:

R₁ is H; at least one of R₂ and R₃ is halogen or —O—R₈; R₄ is H; R₅ is selected from the group consisting of CN, substituted or unsubstituted (C₆-C₁₀)aryl, and —C(═O)—R₁₃; if R₁₃ is —OR₁₅, then at least one of R₂ and R₃ is —O—R₈, wherein R₈ is selected from the group consisting of: H; a saturated or unsaturated, straight or branched, cyclic or acyclic, chiral or achiral hydrocarbyl group having from 1 to 10 carbon atoms, wherein the hydrocarbyl group is substituted with one or more of halogen, hydroxy, (C₁-C₄)alkylcarbonyloxy, or —NR₁₁R₁₂; substituted or unsubstituted (C₆-C₁₀)aryl; substituted or unsubstituted (C₂-C₉)heterocyclyl; and —NH₂;

In one embodiment, R₁ is H; R₂ is —O—CH₃; R₃ is —O—CH₂-Ph; R₄ is H; R₅ is —C(═O)—O—CH₃.

In one embodiment, R₁ is H; R₂ is —O—CH₂-Ph; R₃ is —O—CH₃; R₄ is H; R₅ is —C(═O)—O—CH₃.

In one embodiment, R₁ is H; R₂ is —O—CH₃; R₃ is —O—CH₂-Ph; R₄ is H; R₅ is —C(═O)—O—CH₃.

In one embodiment, R₁ is H; R₂ is F; R₃ is H; R₄ is H; R₅ is CN.

In one embodiment, R₁ is H; R₂ is F; R₃ is H; R₄ is H; R₅ is —C(═O)-Ph-F.

In one embodiment, R₁ is H; R₂ is F; R₃ is F; R₄ is H; R₅ is CN.

In one embodiment, R₁ is H; R₂ is F; R₃ is F; R₄ is H; R₅ is —C(═O)-Ph-F.

In one embodiment, R₁ is H; R₂ is F; R₃ is F; R₄ is H; R₅ is —C(═O)-Ph-O—CH₃.

In one embodiment, R₁ is H; R₂ is F; R₃ is H; R₄ is H; R₅ is —C(═O)-Ph-O—CH₃.

The reaction in Scheme Ia can be achieved in the presence of acid or base catalysts or reagents. Preferably, the base is at least one of K₂CO₃ or NaOH. The reaction in Scheme Ia is preferably carried out in an appropriate solvent, preferably acetone. The reactions in Scheme Ia are typically carried out at a temperature between 0° C. and the reflux temperature of the solvent, which is typically about 50-100° C. The temperature during the reaction in Scheme Ia is preferably room temperature for 10 minutes. The reaction preferably takes place under an inert atmosphere, such as nitrogen gas.

A process is provided in Scheme Ib for the synthesis of compounds according to Formula I, comprising reacting a compound of Formula CC with a compound of Formula DD to form a compound Formula Ib, wherein Formula Ib is an embodiment of Formula I.

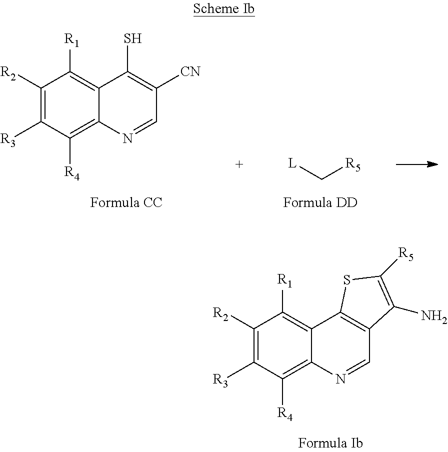

R₁, R₂, R₃, R₄, and R₅ are as defined above.

Particular embodiments of the process in Scheme Ib include those wherein:

L is a leaving group, preferably a halogen, including Cl or Br; R₁ is H; at least one of R₂ and R₃ is —O—R₈; R₄ is H; R₅ is CN, substituted or unsubstituted (C₂-C₉)heterocyclyl, or —C(═O)—R₁₃; and R₈ is independently selected from the group consisting of:
H;
a saturated or unsaturated, straight or branched, cyclic or acyclic, chiral or achiral hydrocarbyl group having from 1 to 10 carbon atoms, wherein the hydrocarbyl group is optionally substituted with one or more of halogen, hydroxy, (C₁-C₄)alkylcarbonyloxy, or —NR₁₁R₁₂, and wherein one or more of the carbon atoms in the hydrocarbyl group is optionally replaced by an oxygen atom (—O—);
substituted or unsubstituted (C₆-C₁₀)aryl;
substituted or unsubstituted (C₂-C₉)heterocyclyl; and
—NH₂;
   R₁₃ is selected from the group consisting of —OR₁₅; and a saturated or unsaturated, straight or branched, cyclic or acyclic, chiral or achiral hydrocarbyl group having from 1 to 10 carbon atoms, wherein the hydrocarbyl group is substituted with one or more of halogens, hydroxy, alkoxy, cyano, carboxy, carbalkoxy, carboxamido, and nitro; and R₁₁ and R₁₂ are each H or NR₁₆ to form a substituted or unsubstituted heterocyclyl ring;

provided that when R₆ is H, R₇ is H, and R₁₃ is —OR₁₅, then at least one of R₂ and R₃ is —O—R₈, wherein R₈ is selected from the group consisting of: H; a saturated or unsaturated, straight or branched, cyclic or acyclic, chiral or achiral hydrocarbyl group having from 1 to 10 carbon atoms, wherein the hydrocarbyl group is substituted with one or more of halogen, hydroxy, (C₁-C₄)alkylcarbonyloxy, or —NR₁₁R₁₂; substituted or unsubstituted (C₆-C₁₀)aryl; substituted or unsubstituted (C₂-C₉)heterocyclyl; and —NH₂;

In one embodiment, R₁ is H; R₂ is —O—CH₃; R₃ is —O—CH₃; R₄ is H; and R₅ is —C(═O)-Ph-F.

In one embodiment, R₁ is H; R₂ is —O—CH₃; R₃ is —O—CH₃; R₄ is H; and R₅ is —C(═O)—CF₃.

In one embodiment, R₁ is H; R₂ is —O—CH₃; R₃ is —O—CH₃; R₄ is H; and R₅ is CN.

In one embodiment, R₁ is H; R₂ is F; R₃ is H; R₄ is H; R₅ is CN.

In one embodiment, R₁ is H; R₂ is F; R₃ is H; R₄ is H; R₅ is —C(═O)-Ph-F.

In one embodiment, R₁ is H; R₂ is F; R₃ is F; R₄ is H; R₅ is CN.

In one embodiment, R₁ is H; R₂ is F; R₃ is F; R₄ is H; R₅ is —C(═O)-Ph-F.

In one embodiment, R₁ is H; R₂ is F; R₃ is F; R₄ is H; R₅ is —C(═O)-Ph-O—CH₃.

In one embodiment, R₁ is H; R₂ is F; R₃ is H; R₄ is H; R₅ is —C(═O)-Ph-O—CH₃.

The reaction in Scheme Ib can proceed in the presence of a base, such a sodium hydroxide or anhydrous potassium carbonate. The reaction in Scheme Ib is preferably carried out in an appropriate solvent, including methanol, acetone, or a mixture thereof. The reactions in Scheme Ib are typically carried out at a temperature between 0° C. and the reflux temperature of the solvent, which is typically about 100° C. The reaction in Scheme Ib is preferably carried out at room temperature for 10 minutes under an inert atmosphere.

A process is provided in Schemes Ic and Id for the synthesis of compounds according to Formula I comprising hydrolysis of a compound of Formula EE (or compound (EE′) to form a compound of Formula FF (or compound FF′), wherein the compound Formula FF (or compound FF′) is further modified to form Formula Ic (or Id), wherein Formula Ic and Id are embodiments of Formula I.

### Schemes Ic and Id

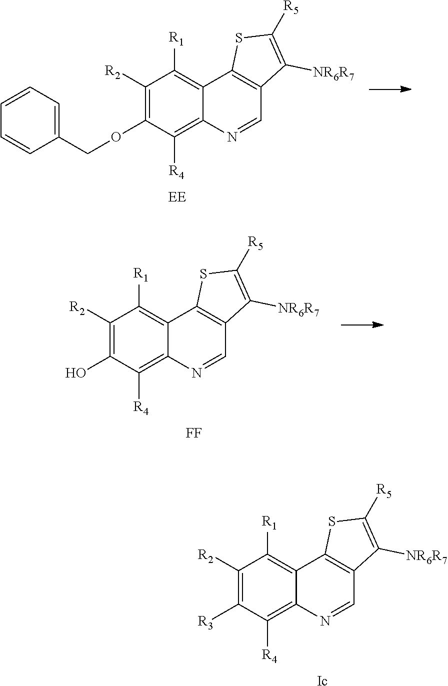

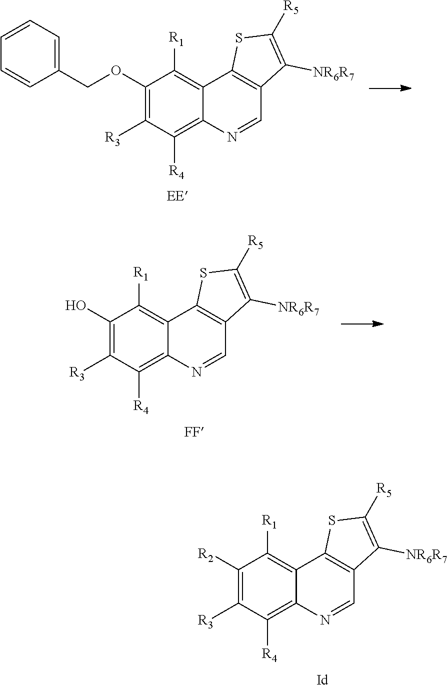

R₁, R₂, R₃, R₄, R₅, R₆, and R₇ are as defined above.

The hydrolysis of Formula EE or EE′ compounds in Schemes Ic and Id, respectively, can proceed in the presence of a fluoronated acid, such as trifluoroacetic acid. The reaction in Schemes Ic and Id is preferably carried out in an appropriate solvent. The reactions in Schemes Ic and Id are typically carried out at a temperature between 0° C. and the reflux temperature of the solvent, which is typically about 100° C. The reaction in Schemes Ic and Id is preferably carried out at room temperature under inert atmosphere, and optionally cooled by pouring the reaction solution into ice water.

In one embodiment, for converting a compound of Formula EE to a compound of Formula FF, R₁ is H; R₂ is —O—CH₃; R₄ is H; R₅ is —C(═O)—O—CH₃; R₆ is H; and R₇ is H.

In one embodiment, for converting a compound of Formula EE′ to a compound of Formula FF′, R₁ is H; R₃ is —O—CH₃; R₄ is H; R₅ is —C(═O)—O—CH₃; R₆ is H; and R₇ is H.

In one embodiment, for converting a compound of Formula FF to a compound of Formula Ic, R₁ is H; R₂ is —O—CH₃; R₃ is —O—R₈; R₄ is H; R₅ is —C(═O)—O—CH₃; R₆ is H; and R₇ is H; and R₈ is selected from the group consisting of: H; a saturated or unsaturated, straight or branched, cyclic or acyclic, chiral or achiral hydrocarbyl group having from 1 to 10 carbon atoms, wherein the hydrocarbyl group is substituted with one or more of halogen, hydroxy, (C₁-C₄)alkylcarbonyloxy, or —NR₁₁R₁₂; substituted or unsubstituted (C₆-C₁₀)aryl; substituted or unsubstituted (C₂-C₉)heterocyclyl; and —NH₂; and R₁₁ and R₁₂ may combine with O to form a substituted or unsubstituted heterocyclyl ring;

In one embodiment, for converting a compound of Formula FF to a compound of Formula Ic, R₁ is H; R₂ is —O—CH₃; R₃ is —O—C(═O)—R₉; R₄ is H; R₅ is —C(═O)—O—CH₃; R₆ is H; and R₇ is H; and R₉ is substituted or unsubstituted (C₂-C₉)heterocyclyl.

In one embodiment, for converting a compound of Formula FF′ to a compound of Formula Id, R₁ is H; R₂ is —O—R₈; R₃ is —O—CH₃; R₄ is H; R₅ is —C(═O)—O—CH₃; R₆ is H; and R₇ is H; and R₈ is selected from the group consisting of H; a saturated or unsaturated, straight or branched, cyclic or acyclic, chiral or achiral hydrocarbyl group having from 1 to 10 carbon atoms, wherein the hydrocarbyl group is substituted with one or more of halogen, hydroxy, (C₁-C₄)alkylcarbonyloxy, or —NR₁₁R₁₂; substituted or unsubstituted (C₆-C₁₀)aryl; substituted or unsubstituted (C₂-C₉)heterocyclyl; and —NH₂; and wherein R₁₁ and R₁₂ may combine with O to form a substituted or unsubstituted heterocyclyl ring;

The reaction of scheme Ic (or Id) can proceed by placing a compound of Formula FF (or Formula FF′) in the presence of R₂-L (or R₃-L), wherein R₂, R₃, and L are as described above. The reaction of scheme Ic or Id can proceed in the presence of a base, such as K₂CO₃/Et₃N, in the presence of an appropriate solvent, such as dimethylformamide (DMF). The reactions are typically carried out at a temperature between 0° C. and the reflux temperature of the solvent, which is typically about 100° C. The reaction is preferably carried out at 60° C. under inert atmosphere for 4 hours, and optionally cooled by pouring the reaction solution into ice water.

A process is provided in Scheme Ie for the synthesis of compounds according to Formula I, comprising reacting a compound of Formula HH with a compound of Formula III to form a compound Formula Ie, wherein Formula Ie is an embodiment of Formula I.

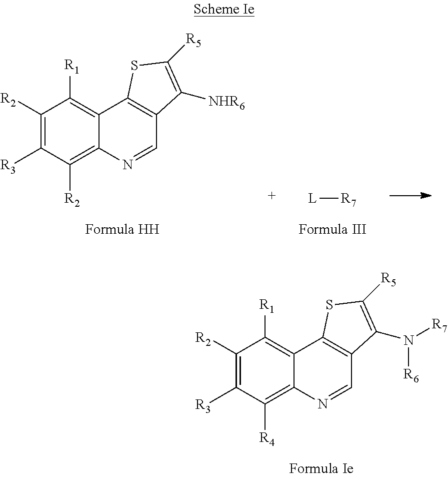

R₁, R₂, R₃, R₄, R₅, R₆, and R₇ are as defined above and L is a leaving group.

Particular embodiments of the process in Scheme Ie include those wherein:

L is a leaving group, preferably a halogen, including Cl or Br; R₁ is H; at least one of R₂ and R₃ is —O—R₈; R₄ is H; R₅ is —C(═O)—R₁₃; R₆ is H; R₇ is —C(═O)—R₁₄, R₈ is independently selected from the group consisting of H; a saturated or unsaturated, straight or branched, cyclic or acyclic, chiral or achiral hydrocarbyl group having from 1 to 10 carbon atoms, wherein the hydrocarbyl group is optionally substituted with one or more of halogen, hydroxy, (C₁-C₄)alkylcarbonyloxy, or —NR₁₁R₁₂, and wherein one or more of the carbon atoms in the hydrocarbyl group is optionally replaced by an oxygen atom (—O—); substituted or unsubstituted (C₆-C₁₀)aryl; substituted or unsubstituted (C₂-C₉)heterocyclyl; and —NH₂; R₁₃ is —OR₁₅; and R₁₄ is selected from the group consisting of:
substituted or unsubstituted (C₂-C₉)heterocyclyl;
—C(═O)-substituted or unsubstituted (C₆-C₁₀)aryl;
—C(═O)-substituted or unsubstituted (C₂-C₉)heterocyclyl;
—C(═O)—NH-substituted or unsubstituted (C₆-C₁₀)aryl;
—C(═O)—NH-substituted or unsubstituted (C₆-C₁₀)arylkyl;
—C(═O)—NH-substituted or unsubstituted (C₂-C₉)heterocyclyl;
—C(═O)-substituted or unsubstituted (C₂-C₉)heterocyclyl-substituted or unsubstituted (C₆-C₁₀)heteroaryl; and
—C(═O)—NH-substituted or unsubstituted (C₆-C₁₀)aryl-substituted or unsubstituted (C₂-C₉)heterocyclyl; and R₁₅ is (C₁-C₆)alkyl.

In one embodiment, R₁ is H; R₂ is —O—CH₃; R₃ is —O—CH₃; R₄ is H; R₅ is —C(═O)—O—CH₃ R₆ is H; and R₇ is —C(═O)-Ph-O—CH₃.

In one embodiment, R₁ is H; R₂ is —O—CH₃; R₃ is —O—CH₃; R₄ is H; R₅ is —C(═O)—O—CH₃ R₆ is H; and R₇ is —C(═O)-Ph-F.

In one embodiment, R₁ is H; R₂ is —O—CH₃; R₃ is —O—CH₃; R₄ is H; R₅ is —C(═O)—O—CH₃ R₆ is H; and R₇ is —C(═O)-Ph.

In one embodiment, R₁ is H; R₂ is —O—CH₃; R₃ is —O—CH₃; R₄ is H; R₅ is —C(═O)—O—CH₃ R₆ is H; and R₇ is —C(═O)-Ph-NO₂.

In one embodiment, R₁ is H; R₂ is —O—CH₃; R₃ is —O—CH₃; R₄ is H; R₅ is —C(═O)—O—CH₃ R₆ is H; and R₇ is —C(═O)-Ph-NH₂.

In one embodiment, R₁ is H; R₂ is —O—CH₃; R₃ is —O—CH₃; R₄ is H; R₅ is —C(═O)—O—CH₃ R₆ is H; and R₇ is —C(═O)—(C₆H₃NO₂Cl).

The reaction in Scheme Ie can take place in presence of an acid chloride and a base, such as Et₃N. The reactions in Scheme Ie are typically carried out in an appropriate solvent, such as dichloromethane (DCM), dimethylformamide (DMF), or a mixture thereof. The reactions in Scheme Ie are typically carried out at a temperature between 0° C. and the reflux temperature of the solvent, which is typically about 100° C. The reaction in Scheme Ie preferably takes place at room temperature for 2-15 hours.

A process is provided in Scheme If for the synthesis of compounds according to Formula I, comprising reacting a compound of Formula JJ with a compound of Formula KK to form a compound Formula If, wherein Formula If is an embodiment of Formula I.

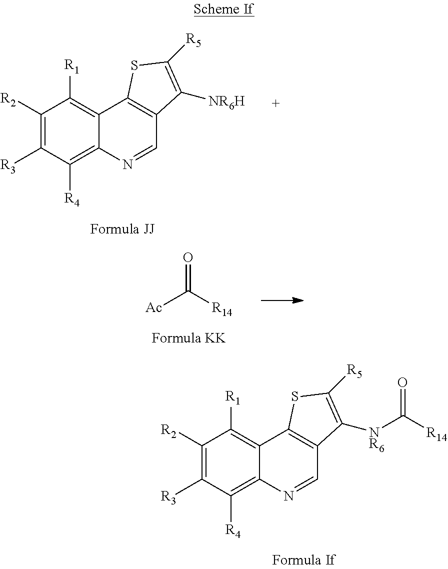

R₁, R₂, R₃, R₄, R₅, R₆, and R₁₄ are as defined above, and Ac is an acyl group, such as alkyl-C(═O)—O— group.

Particular embodiments of the process in Scheme If include those wherein: R₁ is H; at least one of R₂ and R₃ is —O—R₈; R₄ is H; R₅ is —C(═O)—O—CH₃; R₆ is H; and R₈ is independently selected from the group consisting of: H; a saturated or unsaturated, straight or branched, cyclic or acyclic, chiral or achiral hydrocarbyl group having from 1 to 10 carbon atoms, wherein the hydrocarbyl group is optionally substituted with one or more of halogen, hydroxy, (C₁-C₄)alkylcarbonyloxy, or —NR₁₁R₁₂, and wherein one or more of the carbon atoms in the hydrocarbyl group is optionally replaced by an oxygen atom (—O—); substituted or unsubstituted (C₆-C₁₀)aryl; substituted or unsubstituted (C₂-C₉)heterocyclyl; and —NH₂; R₁₄ is selected from the group consisting of: substituted or unsubstituted (C₂-C₉)heterocyclyl; —C(═O)-substituted or unsubstituted (C₆-C₁₀)aryl; —C(═O)-substituted or unsubstituted (C₂-C₉)heterocyclyl; —C(═O)—NH-substituted or unsubstituted (C₆-C₁₀)aryl; —C(═O)—NH-substituted or unsubstituted (C₆-C₁₀)arylkyl; —C(═O)—NH-substituted or unsubstituted (C₂-C₉)heterocyclyl; —C(═O)-substituted or unsubstituted (C₂-C₉)heterocyclyl-substituted or unsubstituted (C₆-C₁₀)heteroaryl; and —C(═O)—NH-substituted or unsubstituted (C₆-C₁₀)aryl-substituted or unsubstituted (C₂-C₉)heterocyclyl; and R₁₅ is selected from the group consisting of H and (C₁-C₆)alkyl.

The reaction in Scheme If can take place in the presence of a compound of Formula KK, wherein Ac is a leaving group, including an ester group. For example the compound KK can be an acid anhydride. The reaction in Scheme If is preferably carried out in an appropriate solvent. The reactions in Scheme If are typically carried out at a temperature between 0° C. and the reflux temperature of the solvent, which is typically about 100° C. The reaction in Scheme If preferably takes place at 60° C. for 2-14 hours.

A process is provided in Scheme Ig for the synthesis of compounds according to Formula I, comprising reacting a compound of Formula LL to form a compound of Formula MM, which can further reacted to form a compound of Formula Ig, wherein Formula Ig is an embodiment of Formula I.

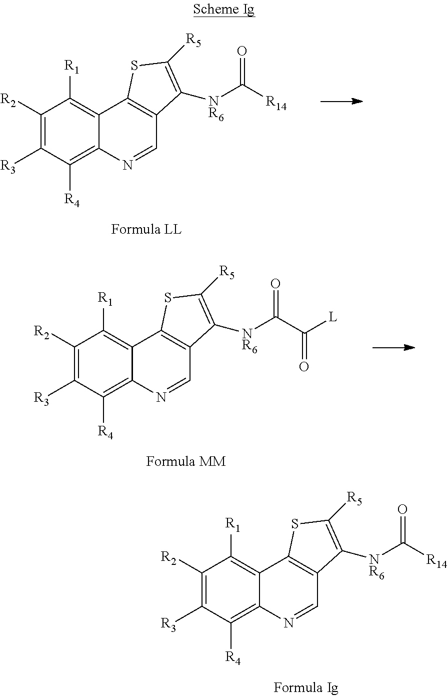

R₁, R₂, R₃, R₄, R₅, R₆, and R₁₄ are as defined above and L is a leaving group, preferably a halogen.

Particular embodiments of the process in Scheme Ig include those wherein:

R₁ is H; at least one of R₂ and R₃ is —O—R₈; R₄ is H; R₅ is —C(═O)—R₁₃; R₆ is H; and R₈ is independently selected from the group consisting of: H; a saturated or unsaturated, straight or branched, cyclic or acyclic, chiral or achiral hydrocarbyl group having from 1 to 10 carbon atoms, wherein the hydrocarbyl group is optionally substituted with one or more of halogen, hydroxy, (C₁-C₄)alkylcarbonyloxy, or —NR₁₁R₁₂, and wherein one or more of the carbon atoms in the hydrocarbyl group is optionally replaced by an oxygen atom (—O—); substituted or unsubstituted (C₆-C₁₀)aryl; substituted or unsubstituted (C₂-C₉)heterocyclyl; and —NH₂; R₁₃ is —OR_{B}; R₁₄ is selected from the group consisting of: substituted or unsubstituted (C₂-C₉)heterocyclyl; —C(═O)-substituted or unsubstituted (C₆-C₁₀)aryl; —C(═O)-substituted or unsubstituted (C₂-C₉)heterocyclyl; —C(═O)—NH-substituted or unsubstituted (C₆-C₁₀)aryl; —C(═O)—NH-substituted or unsubstituted (C₆-C₁₀)arylkyl; —C(═O)—NH-substituted or unsubstituted (C₂-C₉)heterocyclyl; —C(═O)-substituted or unsubstituted (C₂-C₉)heterocyclyl-substituted or unsubstituted (C₆-C₁₀)heteroaryl; and —C(═O)—NH-substituted or unsubstituted (C₆-C₁₀)aryl-substituted or unsubstituted (C₂-C₉)heterocyclyl; and R₁₅ is (C₁-C₆)alkyl.

In one embodiment, R₁ is H; R₂ is —O—CH₃; R₃ is —O—CH₃; R₄ is H; R₅ is —C(═O)—O—CH₃ R₆ is H; and R₁₄ is —C(═O)-[4-methyl piperazinyl].

In one embodiment, R₁ is H; R₂ is —O—CH₃; R₃ is —O—CH₃; R₄ is H; R₅ is —C(═O)—O—CH₃ R₆ is H; and R₁₄ is —C(═O)-[2-morpholino].

In one embodiment, R₁ is H; R₂ is —O—CH₃; R₃ is —O—CH₃; R₄ is H; R₅ is —C(═O)—O—CH₃ R₆ is H; and R₁₄ is —C(═O)-[4-(pyridin-4-yl)piperazin-1-yl].

In one embodiment, R₁ is H; R₂ is —O—CH₃; R₃ is —O—CH₃; R₄ is H; R₅ is —C(═O)—O—CH₃ R₆ is H; and R₁₄ is —C(═O)-[4-(4-methylpiperazin-1-yl)phenyl].

The reaction in Scheme Ig of a compound of Formula LL can take place in the presence of an oxalyl having a leaving group L to produce compound MM, wherein L can be a halogen, including chloride. For example, LL can react in the presence of oxalyl chloride to produce a compound of Formula MM. The conversion of LL to MM is preferably carried out in an appropriate solvent. The reaction in Scheme Ig is typically carried out at a temperature between 0° C. and the reflux temperature of the solvent, which is typically about 100° C. The reaction in Scheme Ig is preferably carried out at from 0° C. to room temperature under an inert atmosphere for 12-15 hours.

The reaction in Scheme Ig of a compound of Formula MM to a Formula Ig in the presence of a base, such as Et₃N, and a nucleophilic amine capable of displacing the leaving group L, such as a primary or secondary amine. The reaction in Scheme Ig is preferably carried out in an appropriate solvent, such as dichloromethane. The reactions in Scheme Ig are typically carried out at a temperature between 0° C. and the reflux temperature of the solvent, which is typically about 100° C. The reaction in Scheme Ig is preferably carried out at room temperature under inert atmosphere for 10-15 hours.

A process is provided in Scheme Ih for the synthesis of compounds according to Formula Ih comprising condensing a compound of Formula NN to form a compound OO, which can be further reacted to provide a compound for Formula Ih, wherein Formula Ih is an embodiment of Formula I.

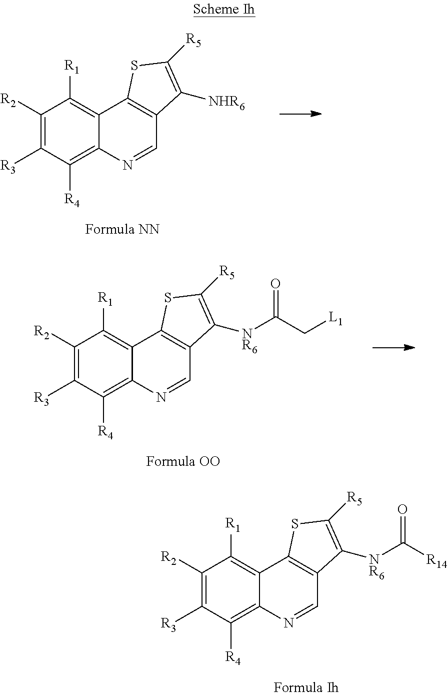

R₁, R₂, R₃, R₄, R₅, R₆, and R₁₄ are as defined above and L₁ is a leaving group, preferably a halogen.

Particular embodiments of the process Scheme Ih include those wherein:

L₁ is a leaving group, preferably a halogen, including Cl or Br; R₁ is H; at least on of R₂ and R₃ is —O—R₈; R₄ is H; R₅ is —C(═O)—R₁₃; R₆ is H; and R₈ is independently selected from the group consisting of H; a saturated or unsaturated, straight or branched, cyclic or acyclic, chiral or achiral hydrocarbyl group having from 1 to 10 carbon atoms, wherein the hydrocarbyl group is optionally substituted with one or more of halogen, hydroxy, (C₁-C₄)alkylcarbonyloxy, or —NR₁₁R₁₂, and wherein one or more of the carbon atoms in the hydrocarbyl group is optionally replaced by an oxygen atom (—O—); substituted or unsubstituted (C₆-C₁₀)aryl; substituted or unsubstituted (C₂-C₉)heterocyclyl; and —NH₂; R₁₃ is —OR₁₅; R₁₄ is selected from the group consisting of substituted or unsubstituted (C₂-C₉)heterocyclyl; —C(═O)-substituted or unsubstituted (C₆-C₁₀)aryl; —C(═O)-substituted or unsubstituted (C₂-C₉)heterocyclyl; —C(═O)—NH-substituted or unsubstituted (C₆-C₁₀)aryl; —C(═O)—NH-substituted or unsubstituted (C₆-C₁₀)arylkyl; —C(═O)—NH-substituted or unsubstituted (C₂-C₉)heterocyclyl; —C(═O)-substituted or unsubstituted (C₂-C₉)heterocyclyl-substituted or unsubstituted (C₆-C₁₀)heteroaryl; and —C(═O)—NH-substituted or unsubstituted (C₆-C₁₀)aryl-substituted or unsubstituted (C₂-C₉)heterocyclyl; and R₁₅ is (C₁-C₆)alkyl.

The reaction in Scheme Ih of compound NN can proceed in the presence of L₁-CH₂—C(═O)-L₂, wherein L₁ and L₂ each independently represents a leaving group, such as a halide, including chloride, and in the presence of a base, such as Et₃N, to form a compound OO. Compound OO can be further reacted with a nucleophile, such as an amine group, in the optional presence of a base to provide the compound to produce the compound Ih. The reaction in Scheme Ih is preferably carried out in an appropriate solvent. The reactions in Scheme Ih are typically carried out at a temperature between 0° C. and the reflux temperature of the solvent, which is typically about 100° C. The reactions in Scheme Ih are typically carried out at a temperature between 0° C. and the reflux temperature of the solvent, which is typically about 100° C., under inert atmosphere for 5-12 hours.

A process is provided in Scheme Ii for the synthesis of compounds according to Formula Ii comprising condensing a compound of Formula PP to form a compound Ii, wherein Formula Ii is an embodiment of Formula I.

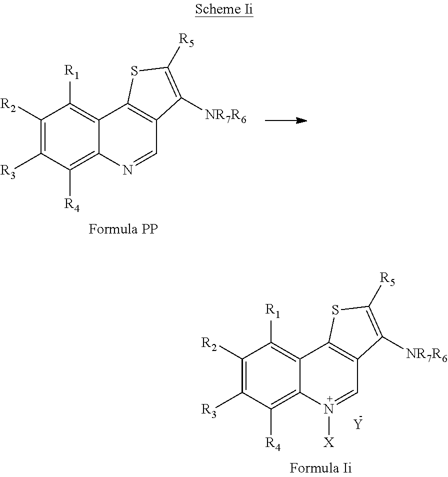

R₁, R₂, R₃, R₄, R₅, R₆, R₇, X and Y are as defined above.

Particular embodiments of the process in Scheme Ii include those wherein:

R₁ is H; R₂ is —O—R₈; R₃ is —O—R₈, R₄ is H; R₅ is —C(═O)—R₁₃; R₆ is H; R₇ is H; R₈ is independently selected from the group consisting of: H; a saturated or unsaturated, straight or branched, cyclic or acyclic, chiral or achiral hydrocarbyl group having from 1 to 10 carbon atoms, wherein the hydrocarbyl group is optionally substituted with one or more of halogen, hydroxy, (C₁-C₄)alkylcarbonyloxy, or —NR₁₁R₁₂, and wherein one or more of the carbon atoms in the hydrocarbyl group is optionally replaced by an oxygen atom (—O—); substituted or unsubstituted (C₆-C₁₀)aryl; substituted or unsubstituted (C₂-C₉)heterocyclyl; and —NH₂; R₁₃ is —OR₁₅; R₁₅ is (C₁-C₆)alkyl; X is selected from the group consisting of (C₁-C₆)alkyl and (C₁-C₆)alkoxyoxo(C₁-C₆)alkyl; and Y is a counterion, preferably halide, including iodide or bromide.

In one embodiment, R₁ is H; R₂ is —O—CH₃; R₃ is —O—CH₃; R₄ is H; R₅ is —C(═O)—R₁₃; R₆ is H; R₇ is H; R₁₃ is —OR₁₅; R₁₅ is (C₁-C₆)alkyl; X is —CH₃; and Y is a counterion, preferably halide, including iodide.

In one embodiment, R₁ is H; R₂ is —O—CH₃; R₃ is —O—CH₃; R₄ is H; R₅ is —C(═O)—R₁₃; R₆ is H; R₇ is H; R₁₃ is —OR₁₅; R₁₅ is (C₁-C₆)alkyl; X is —CH₂—C(═O)—OCH₃; and Y is a counterion, preferably halide, including bromide.

The reaction in Scheme Ii of compound PP can proceed in the presence of XY, where Y is capable of acting as a leaving group for XY. The reaction in Scheme Ii is preferably carried out in an appropriate solvent, ethanol, dichloromethane, dimethyl formamide, and/or sodium acetate. The reactions in Scheme Ii are typically carried out at a temperature between 0° C. and the reflux temperature of the solvent, which is typically about 100° C., under nitrogen for 5-24 hours.

A process is provided in Scheme Ij for the synthesis of compounds according to Formula Ij comprising condensing a compound of Formula QQ to form a compound Ij, wherein Formula Ij is an embodiment of Formula I.

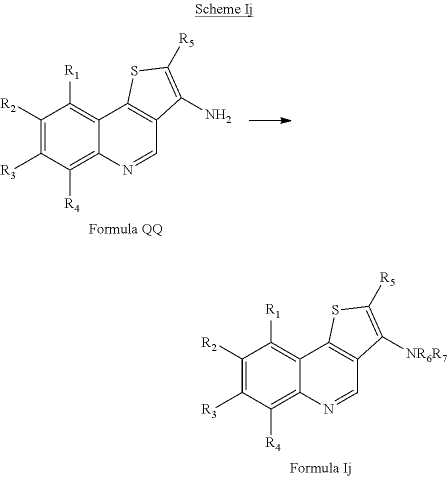

R₁, R₂, R₃, R₄, R₅, R₆, and R₇ are as defined above.

Particular embodiments of the process in Scheme Ij include those wherein:

R₁ is H; R₂ is —O—R₈; R₃ is —O—R₈; R₄ is H; R₅ is —C(═O)—R₁₃; R₆ and R₇ combine to form ═CH—NR₁₇R₁₈; R₈ is independently selected from the group consisting of: H; a saturated or unsaturated, straight or branched, cyclic or acyclic, chiral or achiral hydrocarbyl group having from 1 to 10 carbon atoms, wherein the hydrocarbyl group is optionally substituted with one or more of halogen, hydroxy, (C₁-C₄)alkylcarbonyloxy, or —NR₁₁R₁₂, and wherein one or more of the carbon atoms in the hydrocarbyl group is optionally replaced by an oxygen atom (—O—); substituted or unsubstituted (C₆-C₁₀)aryl; substituted or unsubstituted (C₂-C₉)heterocyclyl; and —NH₂; R₁₃ is —OR₁₅; R₁₅ is (C₁-C₆)alkyl; and R₁₇ and R₁₈ are each independently selected from the group consisting of (C₁-C₆)alkyl.

In one embodiment, R₁ is H; R₂ is —O—CH₃; R₃ is —O—CH₃; R₄ is H; R₅ is —C(═O)—R₁₃; R₆ and R₇ combine to form ═CH—N(CH₃)₂; R₁₃ is —OR₁₅; and R₁₅ is (C₁-C₆)alkyl.

The reaction of compound QQ can proceed in the presence of N,N-dimethylformamide dimethyl acetal. The reaction in Scheme Ij is preferably carried out in an appropriate solvent, such as DMF. The reactions in Scheme Ij are typically carried out at a temperature between 0° C. and the reflux temperature of the solvent, which is typically about 100° C. The reactions in Scheme Ij are preferably carried out at 40° C. for 2 hours under inert atmosphere.

A process is provided in Scheme Ik for the synthesis of compounds according to Formula Ik comprising reacting a compound of Formula RR to form a compound 1k, wherein Formula Ik is an embodiment of Formula I.

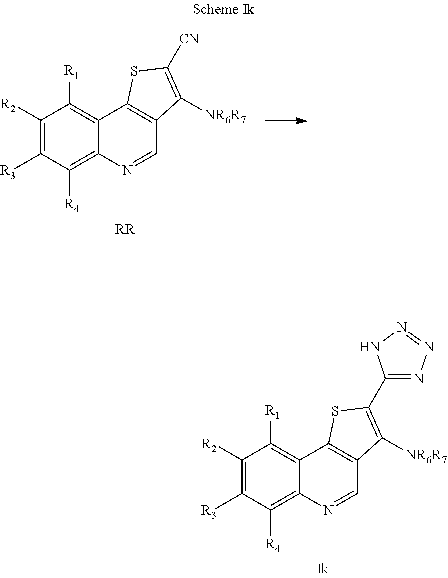

R₁, R₂, R₃, R₄, R₆, and R₇ are as defined above.

Particular embodiments of the process in Scheme Ik include those wherein:

R₁ is H; at least one of R₂ and R₃ is —O—R₈; R₄ is H; R₆ is H; R₇ is H; and R₈ is independently selected from the group consisting of H; a saturated or unsaturated, straight or branched, cyclic or acyclic, chiral or achiral hydrocarbyl group having from 1 to 10 carbon atoms, wherein the hydrocarbyl group is optionally substituted with one or more of halogen, hydroxy, (C₁-C₄)alkylcarbonyloxy, or —NR₁₁R₁₂, and wherein one or more of the carbon atoms in the hydrocarbyl group is optionally replaced by an oxygen atom (—O—); substituted or unsubstituted (C₆-C₁₀)aryl; substituted or unsubstituted (C₂-C₉)heterocyclyl; and —NH₂; and wherein R₁₁ and R₁₂ are H.

In one embodiment, R₁ is H; R₂ is —OCH₃; R₃ is —OCH₃, R₄ is H; R₆ is H; and R₇ is H.

The reaction in Scheme Ik of compound RR can proceed in the presence of an azide, such as sodium azide, and ammonium halide, such as ammonium chloride. The reaction in Scheme Ik is preferably carried out in an appropriate solvent, such as DMF. The reactions in Scheme Ik are typically carried out at a temperature between 0° C. and the reflux temperature of the solvent, which is typically about 100° C. Preferably the reaction in Scheme Ik is carried out at 100° C. for 5 hours under inert atmosphere.

### IV. TREATMENT OF CELLULAR PROLIFERATIVE DISORDERS USING COMPOUNDS OF THE INVENTION

According to another embodiment of the invention, a method of treating an individual suffering from a cellular proliferative disorder, particularly cancer, is provided, comprising administering to said individual an effective amount of at least one compound according to Formula I or Formula II, or a pharmaceutically acceptable salt thereof, either alone, or in combination with a pharmaceutically acceptable carrier.

According to another embodiment of the invention, a method of inducing apoptosis of cancer cells, preferably tumor cells, in an individual afflicted with cancer is provided, comprising administering to said individual an effective amount of at least one compound according to Formula I or Formula II, or a pharmaceutically acceptable salt thereof, either alone, or in combination with a pharmaceutically acceptable carrier.

The compounds according to the invention may be administered to individuals (mammals, including animals and humans) afflicted with a cellular proliferative disorder such as cancer, malignant and benign tumors, blood vessel proliferative disorders, autoimmune disorders, and fibrotic disorders. In a particular embodiment of the invention, the individual treated is a human.

The compounds are believed effective against a broad range of tumor types, including but not limited to the following: ovarian cancer; cervical cancer; breast cancer; prostate cancer; testicular cancer, lung cancer, renal cancer; colorectal cancer; skin cancer; brain cancer; leukemia, including acute myeloid leukemia, chronic myeloid leukemia, acute lymphoid leukemia, and chronic lymphoid leukemia.

More particularly, cancers that may be treated by the compounds, compositions and methods of the invention include, but are not limited to, the following:
cardiac cancers, including, for example sarcoma, e.g., angiosarcoma, fibrosarcoma, rhabdomyosarcoma, and liposarcoma; myxoma; rhabdomyoma; fibroma; lipoma and teratoma;
lung cancers, including, for example, bronchogenic carcinoma, e.g., squamous cell, undifferentiated small cell, undifferentiated large cell, and adenocarcinoma; alveolar and bronchiolar carcinoma; bronchial adenoma; sarcoma; lymphoma; chondromatous hamartoma; and mesothelioma;
gastrointestinal cancer, including, for example, cancers of the esophagus, e.g., squamous cell carcinoma, adenocarcinoma, leiomyosarcoma, and lymphoma; cancers of the stomach, e.g., carcinoma, lymphoma, and leiomyosarcoma; cancers of the pancreas, e.g., ductal adenocarcinoma, insulinoma, glucagonoma, gastrinoma, carcinoid tumors, and vipoma; cancers of the small bowel, e.g., adenocarcinoma, lymphoma, carcinoid tumors, Kaposi's sarcoma, leiomyoma, hemangioma, lipoma, neurofibroma, and fibroma; cancers of the large bowel, e.g., adenocarcinoma, tubular adenoma, villous adenoma, hamartoma, and leiomyoma;
genitourinary tract cancers, including, for example, cancers of the kidney, e.g., adenocarcinoma, Wilm's tumor (nephroblastoma), lymphoma, and leukemia; cancers of the bladder and urethra, e.g., squamous cell carcinoma, transitional cell carcinoma, and adenocarcinoma; cancers of the prostate, e.g., adenocarcinoma, and sarcoma; cancer of the testis, e.g., seminoma, teratoma, embryonal carcinoma, teratocarcinoma, choriocarcinoma, sarcoma, interstitial cell carcinoma, fibroma, fibroadenoma, adenomatoid tumors, and lipoma;
liver cancers, including, for example, hepatoma, e.g., hepatocellular carcinoma; cholangiocarcinoma; hepatoblastoma; angiosarcoma; hepatocellular adenoma; and hemangioma;
bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors;
nervous system cancers, including, for example, cancers of the skull, e.g., osteoma, hemangioma, granuloma, xanthoma, and osteitis deformans; cancers of the meninges, e.g., meningioma, meningiosarcoma, and gliomatosis; cancers of the brain, e.g., astrocytoma, medulloblastoma, glioma, ependymoma, germinoma (pinealoma), glioblastoma multiform, oligodendroglioma, schwannoma, retinoblastoma, and congenital tumors; and cancers of the spinal cord, e.g., neurofibroma, meningioma, glioma, and sarcoma;
gynecological cancers, including, for example, cancers of the uterus, e.g., endometrial carcinoma; cancers of the cervix, e.g., cervical carcinoma, and pre-tumor cervical dysplasia; cancers of the ovaries, e.g., ovarian carcinoma, including serous cystadenocarcinoma, mucinous cystadenocarcinoma, unclassified carcinoma, granulosa-thecal cell tumors, Sertoli-Leydig cell tumors, dysgerminoma, and malignant teratoma; cancers of the vulva, e.g., squamous cell carcinoma, intraepithelial carcinoma, adenocarcinoma, fibrosarcoma, and melanoma; cancers of the vagina, e.g., clear cell carcinoma, squamous cell carcinoma, botryoid sarcoma, and embryonal rhabdomyosarcoma; and cancers of the fallopian tubes, e.g., carcinoma;
hematologic cancers, including, for example, cancers of the blood, e.g., acute myeloid leukemia, chronic myeloid leukemia, acute lymphoblastic leukemia, chronic lymphocytic leukemia, myeloproliferative diseases, multiple myeloma, and myelodysplastic syndrome, Hodgkin's lymphoma, non-Hodgkin's lymphoma (malignant lymphoma) and Waldenström's macroglobulinemia;
skin cancers, including, for example, malignant melanoma, basal cell carcinoma, squamous cell carcinoma, Kaposi's sarcoma, moles dysplastic nevi, lipoma, angioma, dermatofibroma, keloids, psoriasis; and
adrenal gland cancers, including, for example, neuroblastoma.

Cancers may be solid tumors that may or may not be metastatic. Cancers may also occur, as in leukemia, as a diffuse tissue. Thus, the term “tumor cell”, as provided herein, includes a cell afflicted by any one of the above identified disorders.

The compounds are also believed useful in the treatment of non-cancer cellular proliferative disorders, that is, cellular proliferative disorders which are characterized by benign indications. Such disorders may also be known as “cytoproliferative” or “hyperproliferative” in that cells are made by the body at an atypically elevated rate. Non-cancer cellular proliferative disorders believed treatable by compounds according to the invention include, for example: hemangiomatosis in newborn, secondary progressive multiple sclerosis, atherosclerosis, chronic progressive myelodegenerative disease, neurofibromatosis, ganglioneuromatosis, keloid formation, Paget's disease of the bone, fibrocystic disease of the breast, uterine fibroids, Peyronie's disease, Dupuytren's disease, restenosis, benign proliferative breast disease, benign prostatic hyperplasia, X-linked lymphocellular proliferative disorder (Duncan disease), post-transplantation lymphocellular proliferative disorder (PTLD), macular degeneration, and retinopathies, such as diabetic retinopathies and proliferative vitreoretinopathy (PVR)

Other non-cancer cellular proliferative disorders believed treatable by compounds according to the invention include the presence of pre-cancerous lymphoproliferative cells associated with an elevated risk of progression to a cancerous disorder. Many non-cancerous lymphocellular proliferative disorders are associated with latent viral infections such as Epstein-Barr virus (EBV) and Hepatitis C. These disorders often begin as a benign pathology and progress into lymphoid neoplasia as a function of time.

A pharmaceutical composition comprising a pharmaceutically acceptable carrier and a compound, or a pharmaceutically acceptable salt thereof, according to claim 1.

In an aspect of the method, a method of treating an individual suffering from a cellular proliferative disorder, comprising administering to the individual an effective amount of at least one compound, or a pharmaceutically acceptable salt thereof, according to Formula I or Formula II.

In a further aspect of the method, the compound of Formula I is methyl 3-(4-fluorobenzamido)-7,8-dimethoxythieno[3,2-c]quinoline-2-carboxylate, or a pharmaceutically acceptable salt thereof.

In a further aspect of the method, the cellular proliferative disorder is selected from the group consisting of: cancer, malignant and benign tumors, blood vessel proliferative disorders, autoimmune disorders, and fibrotic disorders.

In a further aspect of the method, the cellular proliferative disorder is a cancer selected from the group consisting of: ovarian cancer; cervical cancer; breast cancer; prostate cancer; testicular cancer, lung cancer, renal cancer; colorectal cancer; skin cancer; brain cancer; and leukemia.

In a further aspect of the method, the leukemia is selected from the group consisting of: acute myeloid leukemia, chronic myeloid leukemia, acute lymphoid leukemia, and chronic lymphoid leukemia.

A method of inducing apoptosis of cancer cells in an individual afflicted with cancer, comprising administering to the individual an effective amount of at least one compound, or a pharmaceutically acceptable salt thereof, according to Formula I or Formula II.

According to another embodiment of the invention, a method of treating an individual suffering from a cellular proliferative disorder, particularly cancer, is provided, comprising administering to said individual an effective amount of at least one compound according to Formula II, or a pharmaceutically acceptable salt thereof, either alone, or in combination with a pharmaceutically acceptable carrier:

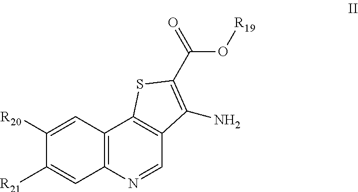

wherein R₁₉ is selected from the group consisting of (C₁-C₆)alkyl; substituted or unsubstituted (C₇-C₁₅)aralkyl; and substituted or unsubstituted (C₆-C₁₀)aryl; and
R₂₀ and R₂₁ are each independently selected from the group consisting of H, halogen, and —O—CH₃.

In another aspect, the invention is a method of administering a compound of Formula II, or a salt thereof wherein: R₁₉ is (C₁-C₆)alkyl.

In another aspect, the invention is a method of administering a compound of Formula II, or a salt thereof wherein: R₁₉ is substituted or unsubstituted (C₇-C₁₅)aralkyl.

In another aspect, the invention is a method of administering a compound of Formula II, or a salt thereof wherein: R₁₉ is substituted or unsubstituted (C₆-C₁₀)aryl.

In another aspect, the invention is a method of administering a compound of Formula II, or a salt thereof wherein: at least one of R₂₀ and R₂₁ is —O—CH₃.

In another aspect, the invention is a method of administering a compound of Formula II, or a salt thereof wherein: at least one of R₂₀ and R₂₁ is H.

In another aspect, the invention is a method of administering a compound of Formula II, or a salt thereof wherein: at least one of R₂₀ and R₂₁ is F.

In an aspect of the method, a method of treating an individual suffering from a cellular proliferative disorder, comprising administering to the individual an effective amount of at least one compound, or a pharmaceutically acceptable salt thereof, according to Formula II.

In a further aspect of the method, the cellular proliferative disorder is selected from the group consisting of: cancer, malignant and benign tumors, blood vessel proliferative disorders, autoimmune disorders, and fibrotic disorders.

In a further aspect of the method, the cellular proliferative disorder is a cancer selected from the group consisting of ovarian cancer; cervical cancer; breast cancer; prostate cancer; testicular cancer, lung cancer, renal cancer; colorectal cancer; skin cancer; brain cancer; and leukemia.

In a further aspect of the method, the leukemia is selected from the group consisting of: acute myeloid leukemia, chronic myeloid leukemia, acute lymphoid leukemia, and chronic lymphoid leukemia.

A method of inducing apoptosis of cancer cells in an individual afflicted with cancer, comprising administering to the individual an effective amount of at least one compound, or a pharmaceutically acceptable salt thereof, according to Formula II.

### V. SALTS OF COMPOUNDS ACCORDING TO THE INVENTION

The compounds of the present invention may take the form of salts when appropriately substituted with groups or atoms capable of forming salts. Such groups and atoms are well known to those of ordinary skill in the art of organic chemistry. The term “salts” embraces addition salts of free acids or free bases which are compounds of the invention. The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts.

All of these salts may be prepared by conventional means from the corresponding compound according to Formula I or Formula II by reacting, for example, the appropriate acid or base with the compound according to Formula I or Formula II. Preferably the salts are in crystalline form, and preferably prepared by crystallization of the salt from a suitable solvent. The person skilled in the art will know how to prepare and select suitable salt forms for example, as described in Handbook of Pharmaceutical Salts: Properties, Selection, and Use By P. H. Stahl and C. G. Wermuth (Wiley-VCH 2002).

### VI. PHARMACEUTICAL COMPOSITIONS

The compounds of the invention may be administered in the form of a pharmaceutical composition, in combination with a pharmaceutically acceptable carrier. The active ingredient or agent in such formulations (i.e. a compound of Formula I or Formula II) may comprise from 0.1 to 99.99 weight percent of the formulation. “Pharmaceutically acceptable carrier” means any carrier, diluent or excipient which is compatible with the other ingredients of the formulation and not deleterious to the recipient.

The active agent is preferably administered with a pharmaceutically acceptable carrier selected on the basis of the selected route of administration and standard pharmaceutical practice. The active agent may be formulated into dosage forms according to standard practices in the field of pharmaceutical preparations. See Alphonso Gennaro, ed., Remington's Pharmaceutical Sciences, 18th Edition (1990), Mack Publishing Co., Easton, Pa. Suitable dosage forms may comprise, for example, tablets, capsules, solutions, parenteral solutions, troches, suppositories, or suspensions.

For parenteral administration, the active agent may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol. Solutions for parenteral administration preferably contain a water soluble salt of the active agent. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or non-aqueous solution, dispersion, suspension or emulsion.

For oral administration, the active agent may be combined with one or more solid inactive ingredients for the preparation of tablets, capsules, pills, powders, granules or other suitable oral dosage forms. For example, the active agent may be combined with at least one excipient such as fillers, binders, humectants, disintegrating agents, solution retarders, absorption accelerators, wetting agents absorbents or lubricating agents. According to one tablet embodiment, the active agent may be combined with carboxymethylcellulose calcium, magnesium stearate, mannitol and starch, and then formed into tablets by conventional tableting methods.

The specific dose of a compound according to the invention to obtain therapeutic benefit for treatment of a cellular proliferative disorder will, of course, be determined by the particular circumstances of the individual patient including the size, weight, age and sex of the patient, the nature and stage of the cellular proliferative disorder, the aggressiveness of the cellular proliferative disorder, and the route of administration of the compound.

For example, a daily dosage from about 0.05 to about 50 mg/kg/day may be utilized, more preferably from about 0.1 to about 10 mg/kg/day. Higher or lower doses are also contemplated as it may be necessary to use dosages outside these ranges in some cases. The daily dosage may be divided, such as being divided equally into two to four times per day daily dosing. The compositions are preferably formulated in a unit dosage form, each dosage containing from about 1 to about 500 mg, more typically, about 10 to about 100 mg of active agent per unit dosage. The term “unit dosage form” refers to physically discrete units suitable as a unitary dosage for human subjects and other mammals, each unit containing a predetermined quantity of active material calculated to produce the desired therapeutic effect, in association with a suitable pharmaceutical excipient.

The pharmaceutical compositions of the present invention may also be formulated so as to provide slow or controlled release of the active ingredient therein using, for example, hydropropylmethyl cellulose in varying proportions to provide the desired release profile, other polymer matrices, gels, permeable membranes, osmotic systems, multilayer coatings, microparticles, liposomes and/or microspheres.

In general, a controlled-release preparation is a pharmaceutical composition capable of releasing the active ingredient at the required rate to maintain constant pharmacological activity for a desirable period of time. Such dosage forms provide a supply of a drug to the body during a predetermined period of time and thus maintain drug levels in the therapeutic range for longer periods of time than conventional non-controlled formulations.

U.S. Pat. No. 5,674,533 discloses controlled-release pharmaceutical compositions in liquid dosage forms for the administration of moguisteine, a potent peripheral antitussive. U.S. Pat. No. 5,059,595 describes the controlled-release of active agents by the use of a gastro-resistant tablet for the therapy of organic mental disturbances. U.S. Pat. No. 5,591,767 describes a liquid reservoir transdermal patch for the controlled administration of ketorolac, a non-steroidal anti-inflammatory agent with potent analgesic properties. U.S. Pat. No. 5,120,548 discloses a controlled-release drug delivery device comprised of swellable polymers. U.S. Pat. No. 5,073,543 describes controlled-release formulations containing a trophic factor entrapped by a ganglioside-liposome vehicle. U.S. Pat. No. 5,639,476 discloses a stable solid controlled-release formulation having a coating derived from an aqueous dispersion of a hydrophobic acrylic polymer. Biodegradable microparticles are known for use in controlled-release formulations. U.S. Pat. No. 5,733,566 describes the use of polymeric microparticles that release antiparasitic compositions.

The controlled-release of the active ingredient may be stimulated by various inducers, for example pH, temperature, enzymes, water, or other physiological conditions or compounds. Various mechanisms of drug release exist. For example, in one embodiment, the controlled-release component may swell and form porous openings large enough to release the active ingredient after administration to a patient. The term “controlled-release component” in the context of the present invention is defined herein as a compound or compounds, such as polymers, polymer matrices, gels, permeable membranes, liposomes and/or microspheres, that facilitate the controlled-release of the active ingredient in the pharmaceutical composition. In another embodiment, the controlled-release component is biodegradable, induced by exposure to the aqueous environment, pH, temperature, or enzymes in the body. In another embodiment, sol-gels may be used, wherein the active ingredient is incorporated into a sol-gel matrix that is a solid at room temperature. This matrix is implanted into a patient, preferably a mammal, having a body temperature high enough to induce gel formation of the sol-gel matrix, thereby releasing the active ingredient into the patient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

### VII. ROUTES OF ADMINISTRATION OF COMPOUNDS AND COMPOSITIONS OF THE INVENTION

The compounds of Formula I or Formula II may be administered by any route, including oral, rectal, sublingual, and parenteral administration. Parenteral administration includes, for example, intravenous, intramuscular, intraarterial, intraperitoneal, intranasal, intravaginal, intravesical (e.g., to the bladder), intradermal, transdermal, topical or subcutaneous administration. Also contemplated within the scope of the invention is the instillation of a drug in the body of the patient in a controlled formulation, with systemic or local release of the drug to occur at a later time. For example, the drug may be localized in a depot for controlled release to the circulation, or for release to a local site of tumor growth.

One or more compounds useful in the practice of the present inventions may be administered simultaneously, by the same or different routes, or at different times during treatment. The compounds may be administered before, along with, or after other medications, including other antiproliferative compounds.

The treatment may be carried out for as long a period as necessary, either in a single, uninterrupted session, or in discrete sessions. The treating physician will know how to increase, decrease, or interrupt treatment based on patient response. According to one embodiment, treatment is carried out for from about four to about sixteen weeks. The treatment schedule may be repeated as required.

### VIII. ISOMERISM IN COMPOUNDS OF THE INVENTION

### A. Optical Isomerism

It will be understood that when or if compounds of the present invention contain one or more chiral centers, the compounds may exist in, and may be isolated as pure enantiomeric or diastereomeric forms or as racemic mixtures. The present invention therefore includes any possible enantiomers, diastereomers, racemates or mixtures thereof of the compounds of the invention.

The isomers resulting from the presence of a chiral center comprise a pair of non-superimposable isomers that are called “enantiomers.” Single enantiomers of a pure compound are optically active, i.e., they are capable of rotating the plane of plane polarized light. Single enantiomers are designated according to the Cahn-Ingold-Prelog system. Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S). In the example below, the Cahn-Ingold-Prelog ranking is A>B>C>D. The lowest ranking atom, D is oriented away from the viewer.

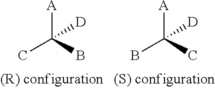

The present invention is meant to encompass diastereomers as well as their racemic and resolved, diastereomerically and enantiomerically pure forms and salts thereof. Diastereomeric pairs may be resolved by known separation techniques including normal and reverse phase chromatography, and crystallization.

“Isolated optical isomer” means a compound which has been substantially purified from the corresponding optical isomer(s) of the same formula. Preferably, the isolated isomer is at least about 80%, more preferably at least 90% pure, even more preferably at least 98% pure, most preferably at least about 99% pure, by weight.

Isolated optical isomers may be purified from racemic mixtures by well-known chiral separation techniques. According to one such method, a racemic mixture of a compound having the structure of Formula I or Formula II, or a chiral intermediate thereof, is separated into 99% wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

### B. Rotational Isomerism

It is understood that due to chemical properties (i.e., resonance lending some double bond character to the C—N bond) of restricted rotation about the amide bond linkage (as illustrated below) it is possible to observe separate rotamer species and even, under some circumstances, to isolate such species. It is further understood that certain structural elements, including steric bulk or substituents on the amide nitrogen, may enhance the stability of a rotamer to the extent that a compound may be isolated as, and exist indefinitely, as a single stable rotamer. The present invention therefore includes any possible stable rotamers of Formula I or Formula II.

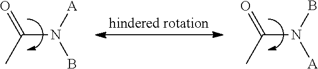

### EXAMPLES

The following non-limiting examples are provided to illustrate the invention. The synthetic procedures described as “general methods” describe what it is believed will be typically effective to perform the synthesis indicated. However, the person skilled in the art will appreciate that it may be necessary to vary the procedures for any given embodiment of the invention. For example, reaction monitoring, such as by using thin layer chromatography, or HPLC may be used to determine the optimum reaction time. Products may be purified by conventional techniques that will vary, for example, according to the amount of side products produced and the physical properties of the compounds. On a laboratory scale, recrystallisation from a suitable solvent, column chromatography, normal or reverse phase HPLC, or distillation are all techniques which may be useful. The person skilled in the art will appreciate how to vary the reaction conditions to synthesize any given compound within the scope of the invention without undue experimentation. See, e.g., Vogel's Textbook of Practical Organic Chemistry, by A. I. Vogel, et al, Experimental Organic Chemistry: Standard and Microscale, by L. M. Harwood et al. (2^{nd} Ed., Blackwell Scientific Publications, 1998), and Advanced Practical Organic Chemistry, by J. Leonard, et al. (2^{nd} Edition, CRC Press 1994).

### Preparative Examples

Scheme 1 was utilized in preparing the following compounds.

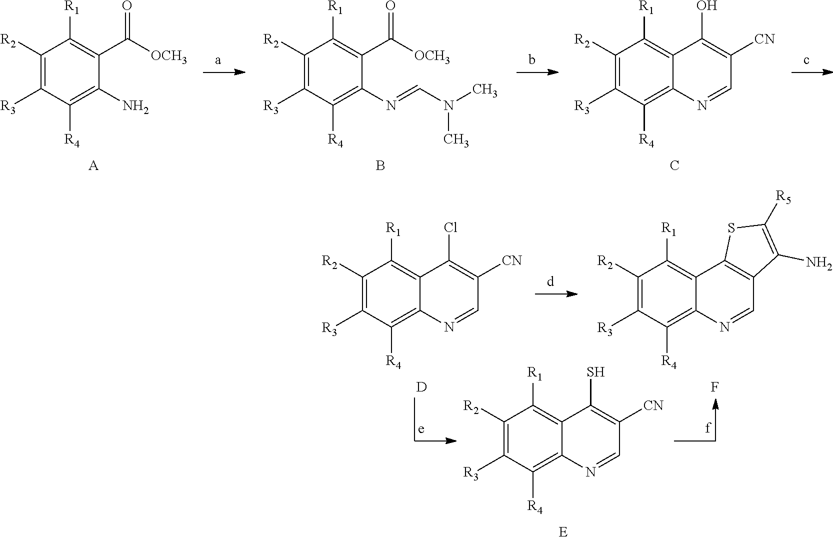

An embodiment of reaction reactions conditions for each step can include: a) N,N-Dimethylformamide dimethyl acetal, DMF, reflux, 2-4 hrs; b) i) n-BuLi, MeCN, THF (dry), −78° C., 2 h, ii) HOAc, −78° C.-rt, 30 min; c) POCl₃, reflux, 2 h; d) R₃CH₂SH, K₂CO₃ (anhydrous), acetone (dry), reflux, overnight; e) i) NH₂CSNH₂, EtOH, 30 min, ii) NaOH, AcOH; f) R₃CH₂X, NaOH, MeOH, 3-15 h.

### General Procedure I

### Preparation of Compound B of Scheme 1: Methyl 2-{[(dimethylamino)methylene]amino}-4,5-di-substituted-benzoate

A solution of A (23.7 mmol) and N,N-dimethylformamide dimethylacetal (6.3 mL, 47.4 mmol) in DMF (25 mL) was heated at reflux under N₂ for 2-4 h. Volatile material was removed and the residue was azeotroped twice with toluene and dried in vacuum (25° C.). The formamidine product B was purple syrup that crystallized on standing. The crude compound was used in the next step without further purification.

### General Procedure II

### Preparation of Compound C of Scheme 1: 4-Hydroxy-6,7-di-substituted-quinoline-3-carbonitrile

A solution of acetonitrile (2.32 mL, 51 mmol) in THF (40 mL) was added over 15 min to a solution of 2.5M n-BuLi in hexane (20 mL, 50 mmol) and THF (30 mL) at −78° C. under N₂. After 20 min, a solution of the crude B in THF (40 mL) was added over 0.5 h. The reaction was stirred at −78° C. for 1 h and then quenched with glacial acetic acid (6.5 mL, 113.5 mmol). The mixture was warmed to 25° C. and stirring was continued for 30 min. The volatile material was removed and the residue was slurried with H₂O (100 mL), collected the separated solid and dried. This material was washed twice with CHCl₃ (50 mL) and dried to give product C.

General Procedure II and General Procedure III were conducted in sequence to prepare the compound C of Preparative Examples 1-3, as follows:

### Preparative Example 1

### 4-Hydroxy-6,7-dimethoxyquinoline-3-carbonitrile (compound C: R¹═R²=methoxy)

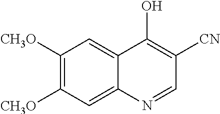

Yield: 2.4 g; m.p.: >300° C.

¹H NMR (DMSO-d₆; 400 MHz): 12.54 (brs, 1H, OH), 8.34 (s, 1H, Ar—H), 7.23 (s, 1H, Ar—H), 6.81 (s, 1H, Ar—H), 3.63 (s, 6H, 2×OCH₃).

LC-MS: (M+H): 231.10.

### Preparative Example 2

### 7-Benzyloxy-4-hydroxy-6-methoxyquinoline-3-carbonitrile (compound C: R¹=methoxy; R²=benzyloxy)

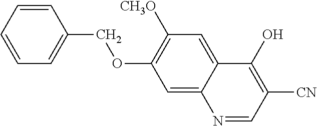

Yield: 2.15 g; m.p.: 292-295° C.

¹H NMR (DMSO-d₆; 400 MHz): 12.40 (brs, 1H, OH), 8.55 (s, 1H, Ar—H), 7.50-7.36 (m, 6H, Ar—H), 7.21 (s, 1H, Ar—H), 5.20 (s, 2H, Ph-CH₂), 3.85 (s, 3H, OCH₃).

LC-MS: (M+H): 307.00.

### Preparative Example 3

### 6-Benzyloxy-4-hydroxy-7-methoxyquinoline-3-carbonitrile (compound C: R¹=benzyloxy; R²=methoxy)

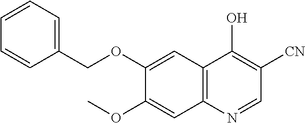 [SMILES: COc1cc2ncc(C#N)c(O)c2cc1OCc1ccccc1]

Yield: 2.05 g; m.p.: 294-298° C.

¹H NMR (DMSO-d₆; 300 MHz): 12.84 (brs, 1H, OH), 8.57 (s, 1H, Ar—H), 7.53 (s, 1H, Ar—H), 7.46-7.32 (m, 5H, Ar—H), 7.13 (s, 1H, Ar—H), 5.17 (s, 2H, Ph-CH₂), 3.87 (s, 3H, OCH₃).

LC-MS: (M+H): 307.00.

### General Procedure III

### Preparation of Compound D of Scheme 1: 4-Chloro-6,7-di-substituted-quinoline-3-carbonitrile

A stirred mixture of C (8.7 mmol) and POCl₃ (95 mmol) was heated at reflux for 2 h under N₂ and the volatile materials were removed under vacuum at about 70° C. The residue was stirred at 0° C. with DCM (100 mL) and H₂O (100 mL) as solid K₂CO₃ was carefully added until the pH 8-9. After stirring for 30 min at 25° C. the organic layer was separated, washed with H₂O, dried, filtered through Celite and concentrated to give analytically pure product D.

### Preparative Example 4

### 4-Chloro-6,7-dimethoxyquinoline-3-carbonitrile (compound D: R¹═R²=methoxy)

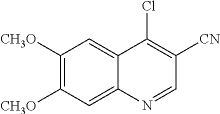

General Procedure III was followed to provide the title compound.

Yield: 1.95 g; m.p.: 218-220° C.

¹H NMR (CDCl₃; 400 MHz): 8.71 (s, 1H, Ar—H), 7.36 (s, 1H, Ar—H), 7.34 (s, 1H, Ar—H), 4.01 (s, 6H, 2×OCH₃).

LC-MS: (M+H): 249.10.

### Preparative Example 5

### 7-Benzyloxy-4-chloro-6-methoxyquinoline-3-carbonitrile (compound D: R¹=methoxy; R²=benzyloxy)

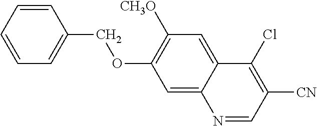

General Procedure III was followed to provide the title compound.

Yield: 1.80 g; m.p.: 182-184° C.

¹H NMR (CDCl₃; 300 MHz): 8.75 (s, 1H, Ar—H), 7.51-7.26 (m, 7H, Ar—H), 5.33 (s, 2H, Ph-CH₂), 4.08 (s, 3H, OCH₃).

LC-MS: (M+H): 325.00.

### Preparative Example 6

### 6-Benzyloxy-4-chloro-7-methoxyquinoline-3-carbonitrile (compound D: R¹=benzyloxy; R²=methoxy)

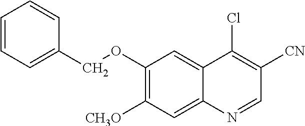

General Procedure III was followed to provide the title compound.

Yield: 1.88 g; m.p.: 176-178° C.

¹H NMR (CDCl₃; 300 MHz): 8.68 (s, 1H, Ar—H), 7.46-7.30 (m, 7H, Ar—H), 5.24 (s, 2H, Ph-CH₂), 4.00 (s, 3H, OCH₃).

LC-MS: (M+H): 325.00.

### General Procedure IV

### Preparation of Compound E of Scheme 1: 4-Mercapto-6,7-di-substituted-quinoline-3-carbonitrile

A solution of D (4.0 mmol) in 30 mL of EtOH was heated to 50° C. and thiourea (4.0 mmol) was added at once. This mixture was stirred vigorously for 30 min and then left to cool slowly to room temperature. The precipitated solid was filtered off, dissolved in 0.2M NaOH solution and stirred for 15 min and filtered. The filtrate was acidified with acetic acid to pH 2, the separated solid filtered and washed with ether (20 mL) to give pure E.

### Preparative Example 7

### 4-Mercapto-6,7-dimethoxyquinoline-3-carbonitrile: (compound E: R¹=methoxy; R²=methoxy)

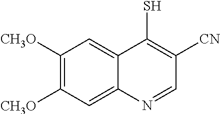

General Procedure IV was followed to provide the title compound.

Yield: 900 mg; m.p.: 238-241° C.

¹H NMR (DMSO-d₆; 600 MHz): 8.53 (s, 1H, Ar—H), 8.09 (s, 1H, Ar—H), 7.12 (s, 1H, Ar—H), 3.94 (s, 3H, OCH₃), 3.91 (s, 3H, OCH₃).

LC-MS: (M+H): 246.90.

### General Procedure V—Methods A and B

### Preparation of Compound F of Scheme 1: 3-Amino-2,7,8-tri-substituted-thieno[3,2-c]quinolone

### Method A

A mixture of alkyl thioglycolate (2.0 mmol) and anhydrous potassium carbonate (8.0 mmol) in dry acetone (20 mL) was stirred at room temperature for 10 min. under N₂. Compound D (2.0 mmol) was added at once to the above contents and the reaction was refluxed over night under N₂. The solvent was removed under reduced pressure and the crude reaction mixture was added to ice cold water (50 mL) with stirring. The separated solid was filtered, washed with water and dried to get pure compound F.

### Method B

4-Mercapto-6,7-dimethoxyquinoline-3-carbonitrile (1.2 mmol) was added at once to the stirred solution of sodium hydroxide/anhydrous potassium carbonate (3.6 mmol) in MeOH/Acetone (dry) (10 mL) and stirring was continued for further 10 min at room temperature. R₅CH₂Cl/R₅CH₂Br (1.2 mmol) was added to the above reaction mixture and the reaction was continued for 3-15 h under N₂. The reaction mixture was poured into ice water (50 mL), the separated solid was filtered, washed with water (10 mL), ether (10 mL) and dried to get pure product F. In some cases the separated solid was filtered, washed with water (10 mL), ether (10 mL) and dried to get pure F.

### Example 1

### Methyl 3-amino-7,8-dimethoxythieno[3,2-c]quinolone-2-carboxylate

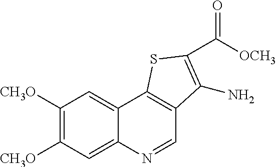

A condensation carried out with 4-chloro-6,7-dimethoxyquinoline-3-carbonitrile (500 mg, 2.0 mmol) and methyl thioglycolate (220 μL, 2.4 mmol) in the presence of anhydrous K₂CO₃ (1.10 g, 8.0 mmol) in acetone (20 mL) according to method A afforded 520 mg of pure title compound.

m.p.: 260-262° C.

¹H NMR (DMSO-d₆; 600 MHz): 9.34 (s, 1H, Ar—H), 7.54 (s, 1H, Ar—H), 7.41 (brs, 2H, NH₂), 7.35 (s, 1H, Ar—H), 3.99 (s, 3H, OCH₃), 3.98 (s, 3H, OCH₃), 3.84 (s, 3H, CO₂CH₃).

LC-MS: (M+H): 319.74.

### Example 2

### Ethyl 3-amino-7,8-dimethoxythieno[3,2-c]quinolone-2-carboxylate

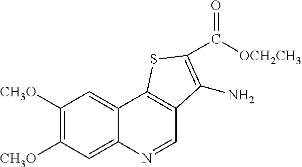

The reaction of 4-chloro-6,7-dimethoxyquinoline-3-carbonitrile (500 mg, 2.0 mmol) with ethyl thioglycolate (260 μL, 2.4 mmol) in the presence of anhydrous K₂CO₃ (1.10 g, 8.0 mmol) in acetone (20 mL) according to General Procedure V, Method A, afforded 500 mg of pure title compound.

m.p.: 215-218° C.

¹H NMR (DMSO-d₆; 400 MHz): 9.32 (s, 1H, Ar—H), 7.51 (s, 1H, Ar—H), 7.41 (brs, 2H, NH₂), 7.32 (s, 1H, Ar—H), 4.33-4.31 (q, 2H, CH₂CH₃), 3.99 (s, 6H, 2×OCH₃), 1.35 (t, 3H, CH₂CH₃).

LC-MS: (M+H): 333.10.

### Example 3

### (3-Amino-7,8-dimethoxythieno[3,2-c]quinolin-2-yl)(4-fluorophenyl)methanone

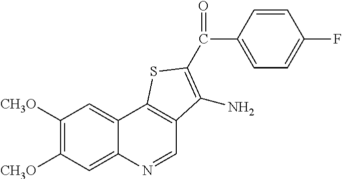

The reaction of 4-mercapto-6,7-dimethoxyquinoline-3-carbonitrile (300 mg, 1.2 mmol) with 4-fluorophenacyl bromide (264 mg, 1.2 mmol) in the presence of NaOH (146 mg, 3.6 mmol) in MeOH (10 mL) afforded 520 mg of pure title compound according to General Procedure V, Method B.

m.p.: 286-288° C.

¹H NMR (DMSO-d₆; 300 MHz): 9.39 (s, 1H, Ar—H), 8.50 (brs, 2H, NH₂), 7.90-7.86 (m, 2H, Ar—H), 7.49 (s, 1H, Ar—H), 7.41-7.35 (m, 2H, Ar—H), 7.25 (s, 1H, Ar—H), 3.94 (s, 3H, OCH₃), 3.92 (s, 3H, OCH₃).

LC-MS: (M+H): 383.10

### Example 4

### Methyl 3-amino-7-benzyloxy-8-methoxythieno[3,2-c]quinolone-2-carboxylate

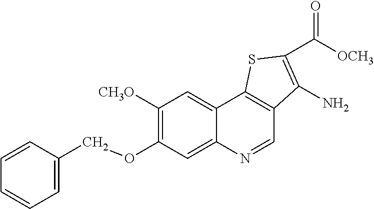

The reaction between 7-benzyloxy-4-chloro-6-methoxyquinoline-3-carbonitrile (300 mg, 0.92 mmol), methyl thioglycolate (100 μL, 1.1 mmol) and an. K₂CO₃ (510 mg, 3.6 mmol) in dry acetone (15 mL) gave 340 mg of pure title compound according to General Procedure V, Method A.

m.p.: 221-223° C.

¹H NMR (DMSO-d₆; 300 MHz): 9.20 (s, 1H, Ar—H), 7.52-7.47 (m, 3H, Ar—H), 7.39-7.32 (m, 3H, Ar—H), 7.31 (brs, 2H, NH₂), 7.22 (s, 1H, Ar—H), 5.24 (s, 2H, Ph-CH₂), 3.91 (s, 3H, OCH₃), 3.77 (s, 3H, CO₂CH₃).

LC-MS: (M+H): 395.10.

### Example 5

### 1-(3-Amino-7,8-dimethoxythieno[3,2-c]quinolin-2-yl)-2,2,2-trifluoroethanone

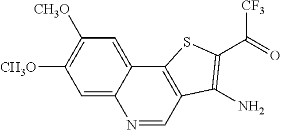

The cyclo condensation of 4-mercapto-6,7-dimethoxyquinoline-3-carbonitrile (200 mg, 0.81 mmol) with 3-bromo-1,1,1-trifluoroacetone (100 μL, 0.97 mmol) in the presence of K₂CO₃ (447 mg, 3.2 mmol) in dry acetone (15 mL) afforded 220 mg of pure title compound by following General Procedure V, Method B.

m.p.: 230-234° C.

¹H NMR (CDCl₃; 300 MHz): 9.60 (s, 1H, Ar—H), 7.56 (s, 1H, Ar—H), 7.44 (s, 1H, Ar—H), 7.19 (brs, 2H, NH₂), 4.04 (s, 3H, OCH₃), 4.01 (s, 3H, OCH₃).

LC-MS: (M+Na): 379.00.

### Example 6

### 1-(3-Amino-7,8-dimethoxythieno[3,2-c]quinolin-2-yl)ethanone

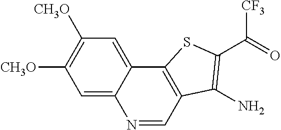

The reaction of 4-mercapto-6,7-dimethoxyquinoline-3-carbonitrile (200 mg, 0.81 mmol), chloro acetone (80 μL, 0.97 mmol) in the presence of NaOH (97 mg, 2.4 mmol) in MeOH (10 mL) gave 210 mg of pure title compound according to General Procedure V, Method B.

m.p.: 260-263° C.

¹H NMR (DMSO-d₆; 300 MHz): 9.30 (s, 1H, Ar—H), 8.05 (brs, 2H, NH₂), 7.47 (s, 1H, Ar—H), 7.25 (s, 1H, Ar—H), 3.94 (s, 6H, 2×OCH₃), 2.38 (s, 3H, COCH₃).

LC-MS: (M+Na): 303.10

### Example 7

### Methyl 3-amino-8-benzyloxy-7-methoxythieno[3,2-c]quinoline-2-carboxylate

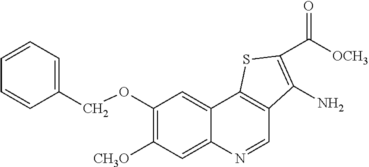

The cyclo condensation of 6-Benzyloxy-4-chloro-7-methoxyquinoline-3-carbonitrile (1.0 g, 3.1 mmol) with methyl thioglycolate (330 μL, 3.7 mmol) in the presence of anhydrous K₂CO₃ (1.71 g, 12.4 mmol) in dry acetone (30 mL) by following General Procedure V, Method A afforded 800 mg of pure title compound.

m.p.: 272-274° C.

¹H NMR (DMSO-d₆; 300 MHz): 9.28 (s, 1H, Ar—H), 7.52-7.50 (m, 3H, Ar—H), 7.42-7.38 (m, 6H, Ar—H & NH₂), 5.29 (s, 2H, Ph-CH₂), 3.94 (s, 3H, OCH₃), 3.80 (s, 3H, CO₂CH₃).

LC-MS: (M+H): 395.10.

### Example 8

### 3-Amino-7,8-dimethoxythieno[3,2-c]quinoline-2-carbonitrile

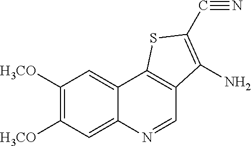 [SMILES: COc1cc2ncc3c(N)c(C#N)sc3c2cc1OC]

The reaction of 4-mercapto-6,7-dimethoxyquinoline-3-carbonitrile (200 mg, 0.81 mmol) with chloroacetonitrile (256 μL, 4 mmol) in the presence of NaOH (97 mg, 2.4 mmol) in MeOH (10 mL) gave 190 mg of pure title compound according to General Procedure V, Method B.

m.p.: 314-316° C.

¹H NMR (DMSO-d₆; 300 MHz): 9.26 (s, 1H, Ar—H), 7.51 (s, 1H, Ar—H), 7.40 (brs, 2H, NH₂), 7.28 (s, 1H, Ar—H), 3.96 (s, 3H, OCH₃), 3.94 (s, 3H, OCH₃).

LC-MS: (M+H): 286.10.

### General Procedure VI

### Preparation of 3-amino-2-substituted-7-hydroxy-8-methoxythieno[3,2-c]quinoline or 3-amino-2-substituted-7-methoxy-8-hydroxythieno[3,2-c]quinoline

A solution of compound 3-amino-2-substituted-7-benzyloxy-8-methoxythieno[3,2-c]quinoline (compound G of Scheme 2) or 3-amino-2-substituted-7-methoxy-8-benzyloxy thieno[3,2-c]quinoline (compound G′ of Scheme 2) (0.5 mmol) in trifluoroacetic acid (10 mL) is refluxed for 5 h under N₂. The reaction is allowed to attain the room temperature, poured in ice-cold water (20 mL) with stirring. The separated solid is filtered, washed with water (10 mL), ether (10 mL) and dried to obtain pure title product.

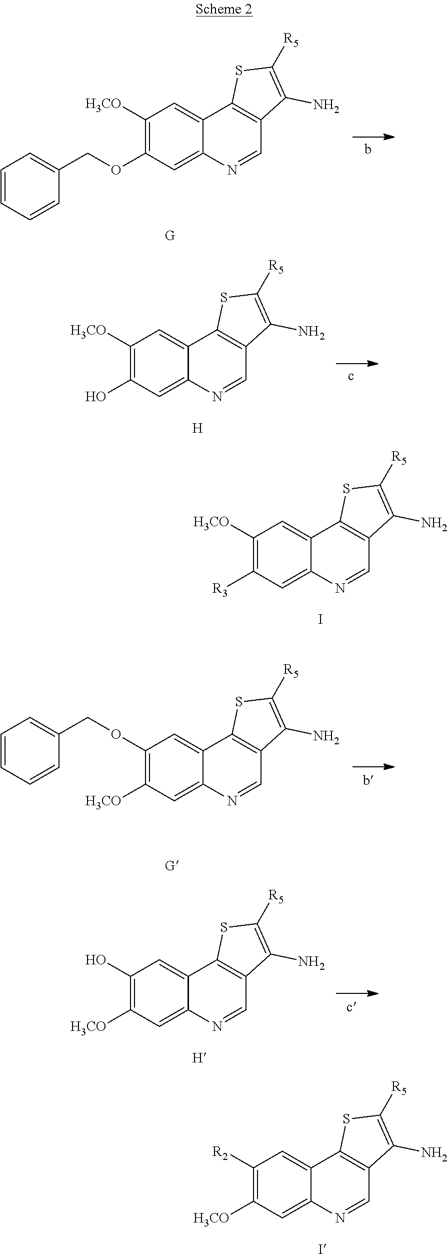

An embodiment of the process shown in scheme 2 can include, a) CF₃CO₂H, reflux, 5 h; b) R₄—Cl, anhydrous K₂CO₃, DMF, 60° C., 4 h (or) a′) CF₃CO₂H, reflux, 5 h; b′) R₄—Cl, Et₃N, DMF, 60° C., 4 h.

### Example 9

### Methyl 3-amino-7-hydroxy-8-methoxythieno[3,2-c]quinoline-2-carboxylate

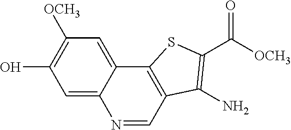 [SMILES: COC(=O)c1sc2c(cnc3cc(O)c(OC)cc32)c1N]

According to General Procedure VI, a solution of methyl 3-amino-7-benzyloxy-8-methoxythieno[3,2-c]quinolone-2-carboxylate (0.5 mmol) in trifluoroacetic acid (10 mL) was refluxed for 5 h under N₂. The reaction was allowed to attain room temperature, and was poured in ice-cold water (20 mL) with stirring. The separated solid was filtered, washed with water (10 mL), ether (10 mL) and dried to give pure title compound.

Yield: 150 mg; m.p.: 244-246° C.

¹H NMR (DMSO-d₆; 300 MHz): 11.33 (brs, 1H, OH), 9.43 (s, 1H, Ar—H), 7.48 (s, 1H, Ar—H), 7.44 (brs, 2H, NH₂), 7.40 (s, 1H, Ar—H), 3.98 (s, 3H, OCH₃), 3.80 (s, 3H, CO₂CH₃).

LC-MS: (M+H): 305.10.

### Example 10

### Methyl 3-amino-8-hydroxy-7-methoxythieno[3,2-c]quinoline-2-carboxylate

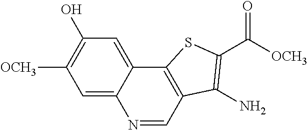 [SMILES: COC(=O)c1sc2c(cnc3cc(OC)c(O)cc32)c1N]

According to General Procedure VI, a solution of methyl 3-amino-8-benzyloxy-7-methoxythieno[3,2-c]quinoline-2-carboxylate (0.5 mmol) in trifluoroacetic acid (10 mL) was refluxed for 5 h under N₂. The reaction was allowed to attain room temperature, and was poured in ice-cold water (20 mL) with stirring. The separated solid was filtered, washed with water (10 mL), ether (10 mL) and dried to give pure title compound.

Yield: 128 mg; m.p.: 277-280° C.

¹H NMR (DMSO-d₆; 300 MHz): 10.23 (brs, 1H, OH), 9.23 (s, 1H, Ar—H), 7.47 (s, 1H, Ar—H), 7.38 (brs, 2H, NH₂), 7.22 (s, 1H, Ar—H), 3.94 (s, 3H, OCH₃), 3.79 (s, 3H, CO₂CH₃).

LC-MS: (M+H): 305.00.

### General Procedure VII

### Preparation of 3-amino-2-substituted-7-substituted-8-methoxythieno[3,2-c]quinoline or 3-amino-2-substituted-7-methoxy-8-substituted-thieno[3,2-c]quinoline

A mixture of 3-amino-2-substituted-7-hydroxy-8-methoxythieno[3,2-c]quinoline (1 mmol) (Scheme 2) and the following are heated at 60° C. for 4 h under N₂: The R₃—Cl (1.5 mmol), anhydrous K₂CO₃/Et₃N (4 mmol) and DMF (8 mL). The reaction mixture is poured into ice-cold water (50 mL) with stirring, filtered and the separated solid is washed with water (10 mL), ether (10 mL) and dried to obtain analytically pure product 3-amino-2-substituted-7-substituted-8-methoxythieno[3,2-c]quinoline.

Similarly, a mixture of 3-amino-2-substituted-7-methoxy-8-hydroxythieno[3,2-c]quinoline and the following are heated at 60° C. for 4 h under N₂: R₂—Cl (1.5 mmol), anhydrous K₂CO₃/Et₃N (4 mmol) and DMF (8 mL). The reaction mixture is poured into ice-cold water (50 mL) with stirring, filtered and the separated solid is washed with water (10 mL), ether (10 mL) and dried to obtain analytically pure product 3-amino-2-substituted-7-methoxy-8-substituted-thieno[3,2-c]quinoline.

### Example 11

### Methyl 3-amino-8-methoxy-7-(3-morpholinopropoxy)thieno[3,2-c]quinoline-2-carboxylate

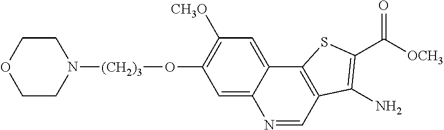

According to General Procedure VII, the reaction of 3-amino-7-hydroxy-8-methoxythieno[3,2-c]quinoline-2-carboxylate (200 mg, 0.66 mmol), 4-(3-chloropropyl)morpholine (161 mg, 0.99 mmol) in the presence of anhydrous K₂CO₃ (363 mg, 2.62 mmol) in DMF (5 mL) gave 210 mg of pure title compound.

m.p.: 117-120° C.

¹H NMR (CDCl₃; 300 MHz): 8.86 (s, 1H, Ar—H), 7.44 (s, 1H, Ar—H), 7.09 (s, 1H, Ar—H), 6.12 (brs, 2H, NH₂), 4.21 (t, 2H), 3.96 (s, 3H, OCH₃), 3.84 (s, 3H, CO₂CH₃), 3.65 (t, 4H), 2.50 (t, 2H), 2.41 (t, 4H), 2.07-2.03 (m, 2H).

LC-MS: (M+H): 432.20.

### Example 12

### Methyl 3-amino-8-methoxy-7-[3-(piperidin-1-yl)propoxy]thieno[3,2-c]quinoline-2-carboxylate

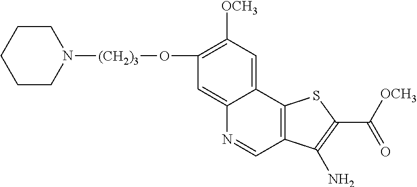

Following General Procedure VII, the reaction of a mixture of 3-amino-7-hydroxy-8-methoxythieno[3,2-c]quinoline-2-carboxylate (200 mg, 0.66 mmol) and 1-(3-chloropropyl)piperidine monohydrochloride (157 mg, 0.79 mmol) in the presence of an. K₂CO₃ (363 mg, 2.62 mmol) in DMF (5 mL) afforded 240 mg of pure title compound.

m.p.: 127-130° C.

¹H NMR (DMSO-d₆; 300 MHz): 9.25 (s, 1H, Ar—H), 7.41 (s, 1H, Ar—H), 7.35 (brs, 2H, NH₂), 7.20 (s, 1H, Ar—H), 3.92 (t, 2H), 3.92 (s, 3H, OCH₃), 3.78 (s, 3H, CO₂CH₃), 2.39-2.36 (m, 2H), 2.34-2.29 (m, 4H), 1.94-1.88 (m, 2H), 1.48-1.44 (m, 4H), 1.35-1.33 (m, 2H).

LC-MS: (M+H): 430.20.

### Example 13

### Methyl 3-amino-8-methoxy-7-[(4-methylpiperazine-1-carbonyl)oxy]thieno[3,2-c]quinoline-2-carboxylate

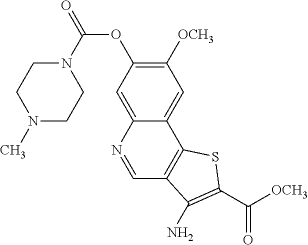 [SMILES: COC(=O)c1sc2c(cnc3cc(OC(=O)N4CCN(C)CC4)c(OC)cc32)c1N]

124 mg of pure title compound was obtained from 3-amino-7-hydroxy-8-methoxythieno[3,2-c]quinoline-2-carboxylate (100 mg, 0.32 mmol) and 4-methyl-1-piperazinecarbonyl chloride (66 μL, 0.49 mmol) in the presence of an. K₂CO₃ (181 mg, 1.3 mmol) in DMF (5 mL) by following General Procedure VII.

m.p.: 147-150° C.

¹H NMR (DMSO-d₆; 300 MHz): 9.43 (s, 1H, Ar—H), 7.67 (s, 1H, Ar—H), 7.52 (brs, 2H, NH₂), 7.39 (s, 1H, Ar—H), 4.25-4.23 (m, 2H), 3.95 (s, 3H, OCH₃), 3.80 (s, 3H, CO₂CH₃), 3.51-3.42 (m, 4H), 3.25-3.23 (m, 2H), 2.48 (s, 3H, N—CH₃).

LC-MS: (M+H): 431.20.

### Example 14

### Methyl 3-amino-8-methoxy-7-(2-morpholinoethoxy)thieno[3,2-c]quinoline-2-carboxylate

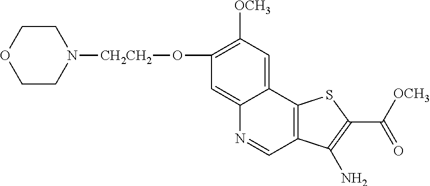

The reaction of 3-amino-7-hydroxy-8-methoxythieno[3,2-c]quinoline-2-carboxylate (200 mg, 0.66 mmol) with 4-(2-chloroethyl)morpholine hydrochloride (183 mg, 0.99 mmol) in the presence of an. K₂CO₃ (365 mg, 2.6 mmol) in DMF (10 mL) according to General Procedure VII gave 160 mg of pure title compound.

m.p.: 158-160° C.

¹H NMR (DMSO-d₆; 300 MHz): 9.16 (s, 1H, Ar—H), 7.58 (s, 1H, Ar—H), 7.37 (brs, 2H, NH₂), 7.29 (s, 1H, Ar—H), 4.26 (t, 2H), 3.95 (s, 3H, OCH₃), 3.86 (s, 3H, CO₂CH₃), 3.57 (t, 4H), 3.32 (t, 2H), 2.77 (t, 2H), 2.36 (t, 2H).

LC-MS: (M+H): 418.20.

### Example 15

### Methyl 3-amino-7-methoxy-8-(3-morpholinopropoxy)thieno[3,2-c]quinoline-2-carboxylate

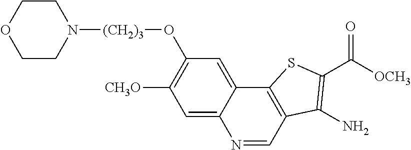

The reaction of 3-amino-8-hydroxy-7-methoxythieno[3,2-c]quinoline-2-carboxylate (200 mg, 0.66 mmol) with 4-(3-chloropropyl)morpholine (161 mg, 0.99 mmol) in the presence of anhydrous K₂CO₃ (363 mg, 2.6 mmol) in DMF (5 mL) according to General Procedure VII resulted in the formation of analytically pure title compound.

Yield: 110 mg; m.p.: 188-190° C.

¹H NMR (DMSO-d₆; 300 MHz): 9.29 (s, 1H, Ar—H), 7.49 (s, 1H, Ar—H), 7.38 (brs, 2H, NH₂), 7.29 (s, 1H, Ar—H), 4.18 (t, 2H), 3.94 (s, 3H, OCH₃), 3.80 (s, 3H, CO₂CH₃), 3.57 (t, 4H), 2.48 (t, 4H), 2.37 (t, 2H), 1.97-1.93 (m, 2H).

LC-MS: (M+H): 432.20.

### General Procedure VIII

### Preparation of 3-(substituted-benzamido)-2,7,8-trisubstituted-thieno[3,2-c]quinoline (compound J, Scheme 3)

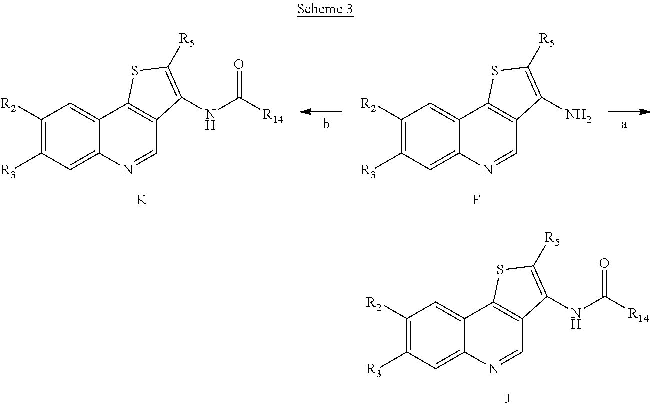

In an embodiment of scheme 3, a) R₁₄(C═O)—Cl, Et₃N, DCM/DSM, rt, 2-15 h; b) (R₁₄—(C═O))2O, 60° C., 2-14 h.

To a solution of 3-amino-2,7,8-tri-substituted-thieno[3,2-c]quinolone (compound F, Scheme 3) (0.63 mmol) in DCM/DMF (15 mL) is added appropriate acid chloride (0.88 mmol) and Et₃N (3.14 mmol) and the reaction mixture is stirred at room temperature for 2-15 h. The separated solid is filtered, washed with water (5 mL), methanol (5 mL) and dried to get pure product 3-(substituted-benzamido)-2,7,8-trisubstituted-thieno[3,2-c]quinoline.

### Example 16

### Methyl 7,8-dimethoxy-3-(4-methoxybenzamido)thieno[3,2-c]quinoline-2-carboxylate

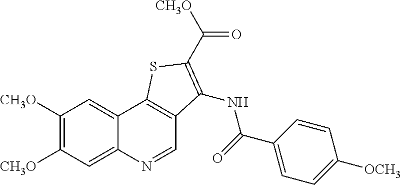

According to General Procedure VIII, the reaction of methyl 3-amino-7,8-dimethoxythieno[3,2-c]quinolone-2-carboxylate (200 mg, 0.63 mmol), 4-methoxybenzoyl chloride (150 mg, 0.88 mmol) in the presence of Et₃N (440 μL, 3.14 mml) in DCM (15 mL) afforded 272 mg of pure title compound.

m.p.: 255-258° C.

¹H NMR (DMSO-d₆; 400 MHz): Poor solubility.

LC-MS: (M+H): 453.10.

### Example 17

### Methyl 3-(2-fluorobenzamido)-7,8-dimethoxythieno[3,2-c]quinoline-2-carboxylate

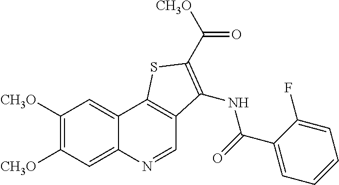

According to General Procedure VIII, the reaction of methyl 3-amino-7,8-dimethoxythieno[3,2-c]quinolone-2-carboxylate (200 mg, 0.63 mmol), 2-fluorobenzoyl chloride (139 mg, 0.88 mmol) in the presence of Et₃N (440 μL, 3.14 mml) in DCM (15 mL) gave pure title compound.

Yield: 260 mg; m.p.: 237-240° C.

¹H NMR (DMSO-d₆; 400 MHz): Solubility problem.

LC-MS: (M+H): 441.10.

### Example 18

### Methyl 3-(4-fluorobenzamido)-7,8-dimethoxythieno[3,2-c]quinoline-2-carboxylate

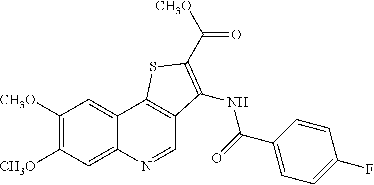

The reaction of a solution of methyl 3-amino-7,8-dimethoxythieno[3,2-c]quinolone-2-carboxylate (200 mg, 0.63 mmol), 4-fluorobenzoyl chloride (102 μL, 0.88 mmol), Et₃N (440 μL, 3.14 mmol) and DCM (15 mL) following General Procedure VIII gave pure title compound.

Yield: 245 mg; m.p.: 196-198° C.

¹H NMR (DMSO-d₆; 300 MHz): 13.06 (brs, 1H, NH), 9.30 (s, 1H, Ar—H), 8.02-7.99 (m, 2H, Ar—H), 7.50-7.47 (m, 2H, Ar—H), 7.45 (s, 1H, Ar—H), 7.42 (s, 1H, Ar—H), 3.98 (s, 3H, OCH₃), 3.95 (s, 3H, OCH₃), 3.84 (s, 3H, CO₂CH₃).

LC-MS: (M+H): 441.10.

### Example 19

### Methyl 3-benzamido-7,8-dimethoxythieno[3,2-c]quinoline-2-carboxylate

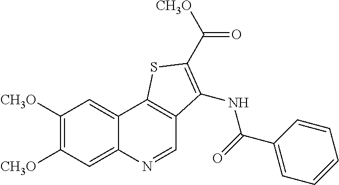

A mixture of methyl 3-amino-7,8-dimethoxythieno[3,2-c]quinolone-2-carboxylate (200 mg, 0.63 mmol), benzoyl chloride (139 mg, 0.88 mmol), Et₃N (440 μL, 3.14 mml) and DCM (15 mL) following General Procedure VIII afforded pure title compound.

Yield: 220 mg; m.p.: 205-208° C.

¹H NMR (DMSO-d₆; 300 MHz): 12.94 (brs, 1H, NH), 9.30 (s, 1H, Ar—H), 7.94-7.91 (m, 2H, Ar—H), 7.51-7.46 (m, 2H, Ar—H), 7.45 (s, 1H, Ar—H), 7.31 (s, 2H, Ar—H), 7.23 (s, 1H, Ar—H), 3.95 (s, 3H, OCH₃), 3.94 (s, 3H, OCH₃), 3.80 (s, 3H, CO₂CH₃).

LC-MS: (M+H): 423.10.

### Example 20

### Methyl 7,8-dimethoxy-3-(2-nitrobenzamido)thieno[3,2-c]quinoline-2-carboxylate

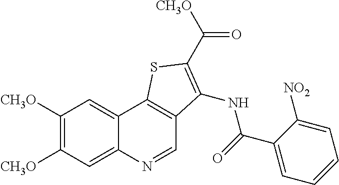

The reaction of methyl 3-amino-7,8-dimethoxythieno[3,2-c]quinolone-2-carboxylate (200 mg, 0.63 mmol), 2-nitrobenzoyl chloride (163 mg, 0.88 mmol) in the presence of Et₃N (440 μL, 3.14 mml) in DMF (15 mL) according to General Procedure VIII gave pure title compound.

Yield: 260; m.p.: 178-181° C.

¹H NMR (DMSO-d₆; 400 MHz): Solubility problem.

LC-MS: (M+H): 468.10.

### Example 21

### Methyl 7,8-dimethoxy-3-(2-aminobenzamido)thieno[3,2-e]quinoline-2-carboxylate

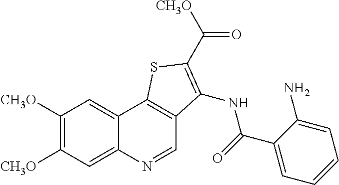

The reaction of methyl 3-amino-7,8-dimethoxythieno[3,2-c]quinolone-2-carboxylate (200 mg, 0.63 mmol) with 2-nitrobenzoyl chloride (163 mg, 0.88 mmol) in the presence of Et₃N (440 μL, 3.14 mml) in DMF (15 mL) gave pure 11e.

Yield: 260; m.p.: 178-181° C.

¹H NMR (DMSO-d₆; 400 MHz): Solubility problem.

LC-MS: (M+H): 468.10.

### Example 22

### Methyl 3-(4-chloro-3-nitrobenzamido)-7,8-dimethoxythieno[3,2-c]quinoline-2-carboxylate

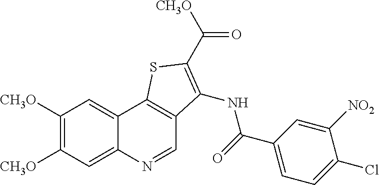

The condensation of methyl 3-amino-7,8-dimethoxythieno[3,2-c]quinolone-2-carboxylate (1.0 g, 3.1 mmol) with 4-chloro-3-nitrobenzoyl chloride (760 mg, 3.4 mmol) in the presence of Et₃N (1.75 mL, 12.5 mmol) in DMF (20 mL) afforded the title compound, according to General Procedure VIII.

Yield: 1.4 g; m.p.: 260-262° C.

¹H NMR (DMSO-d₆; 400 MHz): Solubility problem.

LC-MS: (M+H): 502.10.

### General Procedure IX

### Preparation of 3-acetamido-2,7,8-trisubstituted-thieno[3,2-c]quinoline (compound K, Scheme 3)

A mixture of 3-amino-2,7,8-tri-substituted-thieno[3,2-c]quinolone (compound F, Scheme 3) (0.63 mmol) and appropriate substituted or unsubstituted acid anhydride (10 mL) are heated at 60° C. for 2-14 h. The reaction mixture is allowed to attain room temperature, filtered the solid, washed with ether (10 mL) and dried to obtain pure product.

### Example 23

### Methyl 7,8-dimethoxy-3-(2,2,2-trifluoroacetamido)thieno[3,2-c]quinoline-2-carboxylate

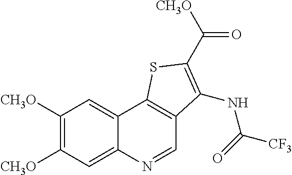

The reaction of methyl 3-amino-7,8-dimethoxythieno[3,2-c]quinolone-2-carboxylate (200 mg, 0.63 mmol) with trifluoroacetic anhydride (10 mL) according to General Procedure IX resulted in the formation of pure title compound.

Yield: 245 mg; m.p.: 224-230° C.

¹H NMR (DMSO-d₆; 400 MHz): 11.91 (brs, 1H, NH), 9.15 (s, 1H, Ar—H), 7.59 (s, 1H, Ar—H), 7.58 (s, 1H, Ar—H), 4.01 (s, 3H, OCH₃), 3.98 (s, 3H, OCH₃), 3.89 (s, 3H, CO₂CH₃).

LC-MS: (M+H): 415.10.

### General Procedure X

### Preparation of 3-(2-chloro-2-oxacetamido)-2,7,8-trisubstituted-thieno[3,2-c]quinoline (compound L, Scheme 4)

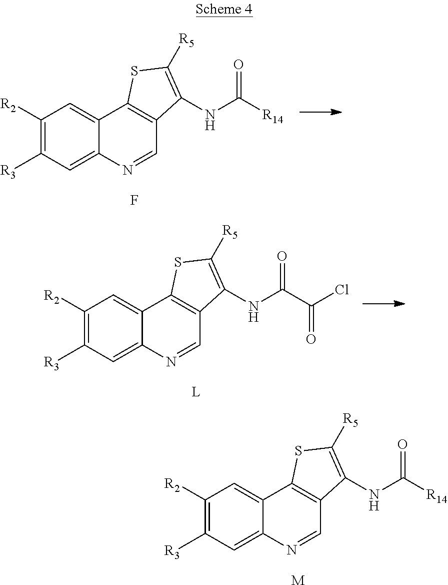

Oxalyl chloride (4 mL) is added to 3-amino-2,7,8-tri-substituted-thieno[3,2-c]quinolone (compound F, Scheme 4) (200 mg, 0.63 mmol) at 0° C. under N₂ and continue stirring at 0° C. to room temperature for another 12-15 h. After completion of the reaction oxalyl chloride is removed under reduced pressure and the crude compound is dried in vacuum and the same was used further without purification.

### Example 24

### Methyl 3-(2-chloro-2-oxoacetamido)-7,8-dimethoxythieno[3,2-c]quinoline-2-carboxylate

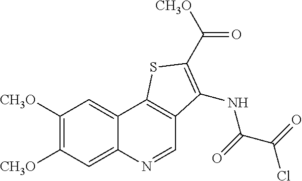

Oxalyl chloride (4 mL) was added to methyl 3-amino-7,8-dimethoxythieno[3,2-c]quinolone-2-carboxylate (200 mg, 0.63 mmol) at 0° C. under N₂ and continue stirring at 0° C. to room temperature for another 12-15 h. After completion of the reaction oxalyl chloride was removed under reduced pressure and the crude compound was dried in vacuum and the same was used further without purification.

### General Procedure XI

### Preparation of 3-(2-substituted-2-oxoacetamido)-2,7,8-trisubstituted-thieno[3,2-c]quinoline (compound M, Scheme 4)

Crude 3-(2-chloro-2-oxacetamido)-2,7,8-trisubstituted-thieno[3,2-c]quinoline (0.63 mmol) was dissolved in dry DCM (5 mL) and a solution of appropriate amine (0.75 mmol) and Et₃N (0.75 mmol) dissolved in DCM (5 mL) was added at room temperature under N₂ and the stirring was continued for 10-15 h. The solvent was removed under reduced pressure and the residue was treated with ice-cold water (30 mL). The separated solid was filtered, washed with ether (10 mL) and dried to get analytically pure product.

### Example 25

### Methyl 7,8-dimethoxy-3-[2-(4-methylpiperazin-1-yl)-2-oxoacetamido]thieno[3,2-c]quinoline-2-carboxylate

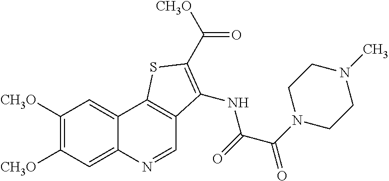

The reaction of methyl 3-(2-chloro-2-oxoacetamido)-7,8-dimethoxythieno[3,2-c]quinoline-2-carboxylate (256 mg, 0.63 mmol) with 1-methylpiperazine (84 μL, 0.75 mmol) in the presence of Et₃N (100 μL, 0.75 mmol) in DCM (10 mL) according to General Procedure XI gave 170 mg of pure title compound.

m.p.: 238-241° C.

¹H NMR (DMSO-d₆; 300 MHz): 11.53 (brs, 1H, NH), 9.07 (s, 1H, Ar—H), 7.52 (s, 2H, Ar—H), 4.46-4.44 (m, 2H), 3.96 (s, 6H, 2×OCH₃), 3.87 (s, 3H, CO₂CH₃), 3.52-3.50 (m, 6H), 2.79 (s, 3H, N—CH₃).

LC-MS: (M+H): 460.20.

### Example 26

### Methyl 7,8-dimethoxy-3-(2-morpholino-2-oxoacetamido)thieno[3,2-c]quinoline-2-carboxylate

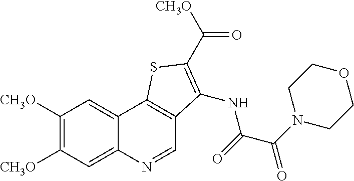

The compound methyl 3-(2-chloro-2-oxoacetamido)-7,8-dimethoxythieno[3,2-c]quinoline-2-carboxylate (256 mg, 0.63 mmol) on treatment with morpholine (66 μL, 0.75 mmol) in the presence of Et₃N (100 μL 0.75 mmol) according to General Procedure XI gave 185 mg of pure title compound.

m.p.: 232-234° C.

¹H NMR (DMSO-d₆; 300 MHz): 11.38 (brs, 1H, NH), 9.05 (s, 1H, Ar—H), 7.55 (s, 2H, Ar—H), 3.97 (s, 6H, 2×OCH₃), 3.88 (s, 3H, CO₂CH₃), 3.70 (t, 4H), 2.07 (t, 4H).

LC-MS: (M+H): 460.20.

### Example 27

### Methyl 7,8-dimethoxy-3-{2-oxo-2-[4-(pyridin-4-yl)piperazin-1-yl]acetamido}thieno[3,2-c]quinoline-2-carboxylate

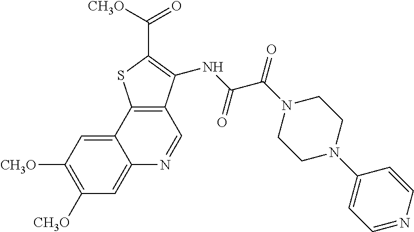

The reaction of methyl 3-(2-chloro-2-oxoacetamido)-7,8-dimethoxythieno[3,2-c]quinoline-2-carboxylate (256 mg, 0.63 mmol) with 1-(4-pyridyl)piperazine (103 mg, 0.63 mmol) in the presence of Et₃N (100 μL, 0.75 mmol) in DCM (10 mL) afforded 220 mg of pure title compound.

m.p.: 245-248° C.

¹H NMR (DMSO-d₆; 300 MHz): 11.62 (brs, 1H, NH), 9.25 (s, 1H, Ar—H), 8.24 (s, 2H, Ar—H), 7.64 (d, 2H, Ar—H, J=6.8 Hz), 7.17 (d, 2H, Ar—H, J=6.8 Hz), 3.96 (s, 3H, OCH₃), 3.92 (s, 3H, OCH₃), 3.84 (s, 3H, CO₂CH₃), 3.83-3.73 (m, 8H).

LC-MS: (M+H): 536.20.

### Example 28

### Methyl 7,8-dimethoxy-3-(2-{[4-(4-methylpiperazin-1-yl)phenyl]amino}-2-oxoacetamido)thieno[3,2-c]quinoline-2-carboxylate

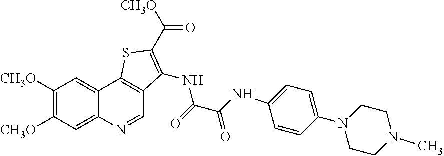

A mixture of methyl 3-(2-chloro-2-oxoacetamido)-7,8-dimethoxythieno[3,2-c]quinoline-2-carboxylate (256 mg, 0.63 mmol), 4-(4-methylpiperazino) aniline (120 mg, 0.63 mmol) and Et₃N (100 μL, 0.75 mmol) in DCM (10 mL) according to General Procedure XI gave 190 mg of pure title compound.

m.p.: 240-246° C.

¹H NMR (DMSO-d₆; 300 MHz): Insoluble.

LC-MS: (M+H): 564.20.

### General Procedure XII

### Preparation of 3-(2-chloroacetamido)-2,7,8-trisubstituted-thieno[3,2-c]quinoline (compound N, Scheme 4)

To a solution of 3-amino-2,7,8-tri-substituted-thieno[3,2-c]quinolone (compound F, Scheme 5) (0.63 mmol) in 1,4-dioxan (5 mL) and Et₃N (100 μL), is added chloroacetyl chloride (1 mL) drop-wise with stirring under N₂. The reaction mixture is refluxed for 5 h, the solvent is evaporated in vacuo and the residual solid is stirred by adding ice-cold water (20 mL). The precipitated solid is filtered, washed with water (5 mL), ether (5 mL), and dried.

### Example 29

### Methyl 3-(2-chloroacetamido)-7,8-dimethoxythieno[3,2-c]quinoline-2-carboxylate

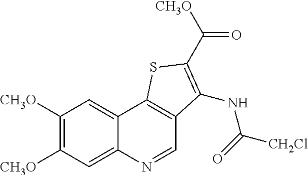

According to General Procedure XII, to a solution of methyl 3-amino-7,8-dimethoxythieno[3,2-c]quinolone-2-carboxylate (0.63 mmol) in 1,4-dioxan (5 mL) and Et₃N (100 μL), was added chloroacetyl chloride (1 mL) drop-wise with stirring under N₂. The reaction mixture was refluxed for 5 h, evaporated the solvent in vacuo and the residual solid was stirred by adding ice-cold water (20 mL). The precipitated solid was filtered, washed with water (5 mL), ether (5 mL), dried and used as is for the next example.

### General Procedure XIII

### Preparation of 3-(2-(substituted)acetamido)-2,7,8-trisubstituted-thieno[3,2-c]quinoline (compound P, Scheme 5)

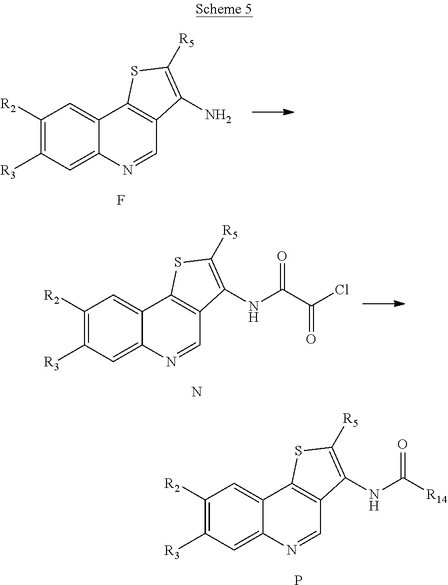

To a stirred mixture of 3-(2-chloroacetamido)-2,7,8-trisubstituted-thieno[3,2-c]quinoline (0.38 mmol) in 1,4-dioxane (10 mL) is added an appropriate amine (0.57 mmol), Et₃N (2 mL) and refluxed for 5 h under N₂. The mixture is allowed to attain room temperature, filtered the solid, washed with water (10 mL), ether (10 mL) and dried to obtain analytically pure 3-(2-(substituted)acetamido)-2,7,8-trisubstituted-thieno[3,2-c]quinoline.

### General Procedure XIV

### Preparation of Compound Q of Scheme 6: 3-Amino-2,5,7,8-tetra-substituted-thieno[3,2-c]quinolin-5-ium salt

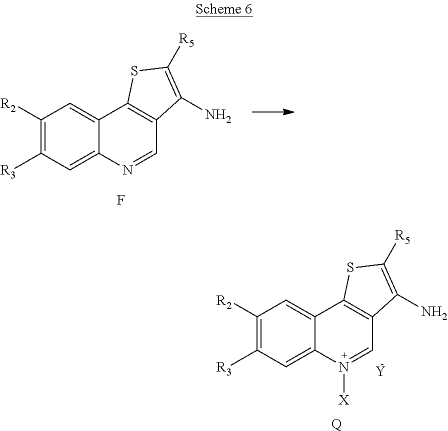

A 3-amino-2,7,8-tri-substituted-thieno[3,2-c]quinolone (compound F, Scheme 6) is reacted with the an appropriate compound XY where Y is a halogen to form a 3-amino-2,5,7,8-tetra-substituted-thieno[3,2-c]quinolin-5-ium salt (Compound Q of Scheme 6).

### Example 30

### Preparation of 3-amino-7,8-dimethoxy-2-(methoxycarbonyl)-5-methylthieno[3,2-c]quinolin-5-ium iodide

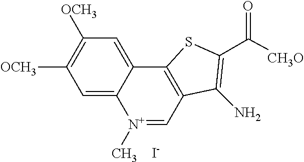

In accordance with General Procedure XIV, methyl 3-amino-7,8-dimethoxythieno[3,2-c]quinolone-2-carboxylate (100 mg, 0.31 mmol) was dissolved in a solution of DCM (2 mL) and DMF (0.2 mL), and MeI (0.5 mL) was added. The reaction flask was sealed, and the mixture was stirred overnight at 40° C. The contents of the flask were brought to attain room temperature, filtered the solid, washed with DCM (5 mL) and dried to give 110 mg of pure title compound.

m.p.: 249-251° C.

¹H NMR (DMSO-d₆; 600 MHz): 9.83 (s, 1H, Ar—H), 7.69 (s, 2H, Ar—H), 7.64 (brs, 2H, NH₂), 4.52 (s, 3H, N—CH₃), 4.15 (s, 3H, OCH₃), 4.07 (s, 3H, OCH₃), 3.86 (s, 3H, CO₂CH₃).

LC-MS: (M+ion): 333.38.

### Example 31

### Preparation of 3-amino-7,8-dimethoxy-5-(2-methoxy-2-oxoethyl)-2-(methoxycarbonyl)thieno[3,2-c]quinolin-5-ium bromide

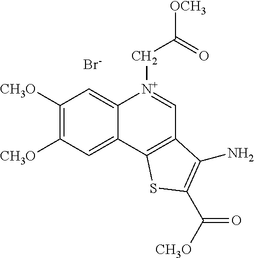

In accordance with General Procedure XIV, a mixture of methyl 3-amino-7,8-dimethoxythieno[3,2-c]quinolone-2-carboxylate (200 mg, 0.63 mmol), methyl bromoacetate (240 μL, 2.5 mmol), NaOAc (206 mg, 2.5 mmol) and EtOH (10 mL) were refluxed for 24 h under N₂. The contents were allowed to attain room temperature and cold water (10 mL) was added. The solid separated was filtered, washed with water (5 mL), ether (5 mL), MeOH (5 mL) and dried to get analytically pure product.

Yield: 160 mg; m.p.: 218-220° C.

¹H NMR (DMSO-d₆; 600 MHz): 9.93 (s, 1H, Ar—H), 7.74 (s, 1H, Ar—H), 7.65 (brs, 3H, Ar—H & NH₂), 6.06 (s, 2H, N—CH₂CO), 4.27 (s, 3H, OCH₃), 4.08 (s, 6H, 2×OCH₃), 3.87 (s, 3H, CO₂CH₃).

### Example 32

### (E)-methyl 3-(((dimethylamino)methylene)amino)-7,8-dimethoxythieno[3,2-c]quinoline-2-carboxylate

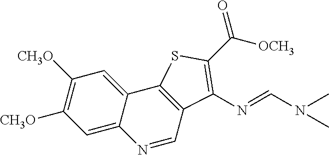

Methyl 3-amino-7,8-dimethoxythieno[3,2-c]quinolone-2-carboxylate (200 mg, 0.63 mmol) was dissolved in dry DMF (3 mL) and N,N-dimethylformamide dimethyl acetal (920 μL, 7 mmol) was added. The resulting solution was stirred at 40° C. for 2 h under N₂. The solution was then evaporated to dryness under vacuum, poured into ice-cold water (20 mL). The separated solid was filtered, washed with ether (5 mL) and dried to get the title compound as pure product.

Yield: 220 mg: m.p.: 173-175° C.

¹H NMR (DMSO-d₆; 600 MHz): 8.94 (s, 1H, Ar—H), 7.99 (s, 1H, ═CH), 7.52 (s, 1H, Ar—H), 7.40 (s, 1H, Ar—H), 3.98 (s, 3H, OCH₃), 3.95 (s, 3H, OCH₃), 3.78 (s, 3H, CO₂CH₃), 3.10 (s, 6H, 2×N—CH₃).

LC-MS: (M+H): 374.10.

### Example 33

### 7,8-dimethoxy-2-(1H-tetrazol-5-yl)thieno[3,2-e]quinolin-3-amine

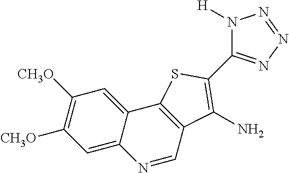

To a solution of 3-amino-7,8-dimethoxythieno[3,2-c]quinoline-2-carbonitrile (100 mg, 0.35 mmol) in DMF (4 mL), sodium azide (27 mg, 0.42 mmol) and ammonium chloride (22 mg, 0.42 mmol) were added and the mixture was heated to 100° C. for 5 h under N₂. After being allowed to cool to room temperature the mixture was poured into ice-water and acidified with 3N HCl to pH 4-5. The precipitate was filtered, washed thoroughly with H₂O (10 mL), MeOH (10 mL) and dried to afford the title compound.

Yield: 104 mg; m.p.: 252-255° C.

LC-MS: (M+H): 329.20.

### General Procedure XV

### Preparation of Compound R of Scheme 7: 3-Amino-2,5,7,8-tetra-substituted-thieno[3,2-c]quinolin-5-ium salt

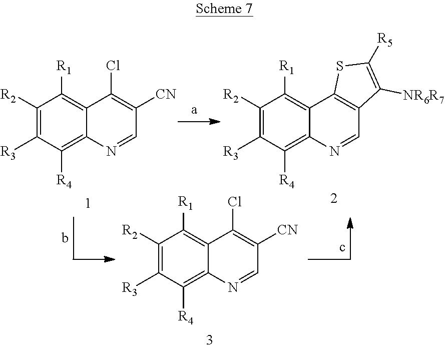

a) R₅CH₂SH, K₂CO₃ (an), acetone (dry), reflux, overnight;
b) i) NH₂CSNH₂, EtOH, 30 min; ii) 10% Aq. NaOH, 3N HCl

### c) R₅CH₂X, NaOH

### Preparative Example 8

### 6-Fluoro-4-mercaptoquinoline-3-carbonitrile (3a)

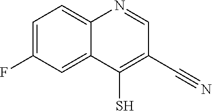 [SMILES: N#Cc1cnc2ccc(F)cc2c1S]

A solution of 4-chloro-6-fluoroquinoline-3-carbonitrile (1a) (1 g, 4.8 mmol) in 20 mL of EtOH was heated to 50° C. and thiourea (368 mg, 4.8 mmol) was added at once. This mixture was stirred vigorously for 30 min and then left to cool slowly to room temperature. The precipitated solid was filtered off, dissolved in 10% aq. NaOH solution (20 mL) and stirred for 15 min and filtered. The filtrate was acidified with 3N HCl up to pH 4, filtered the separated solid and washed with water (20 mL) and ether (20 mL) to give pure 3a.

Yield: 940 mg; m.p.: 288-290° C.

¹H NMR (DMSO-d₆; 600 MHz): 8.76 (s, 1H, Ar—H), 8.33-8.31 (m, 1H, Ar—H), 7.85-7.79 (m, 2H, Ar—H).

LC-MS: (M+H): 205.04.

### Preparative Example 9

### 6,7-Difluoro-4-mercaptoquinoline-3-carbonitrile (3b)

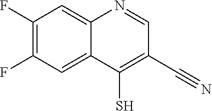 [SMILES: N#Cc1cnc2cc(F)c(F)cc2c1S]

A solution of 4-chloro-6,7-difluoroquinoline-3-carbonitrile (1b) (1 g, 4.5 mmol) in 20 mL of EtOH was heated to 50° C. and thiourea (338 mg, 4.5 mmol) was added at once. This mixture was stirred vigorously for 30 min and then left to cool slowly to room temperature. The precipitated solid was filtered off, dissolved in 10% aq. NaOH solution (20 mL) and stirred for 15 min and filtered. The filtrate was acidified with 3N HCl up to pH 4, filtered the separated solid and washed with water (20 mL) and ether (20 mL) to give pure 3b.

Yield: 900 mg; m.p.: 254-256° C.

¹H NMR (DMSO-d₆; 600 MHz): 8.77 (s, 1H, Ar—H), 8.52-8.48 (m, 1H, Ar—H), 7.77-7.74 (m, 1H, Ar—H).

LC-MS: (M+H): 223.02.

### General Method for the Preparation of 2 from Scheme 7

### Method A

A mixture of methyl thioglycolate (2.0 mmol) and anhydrous potassium carbonate (8.0 mmol) in dry acetone (20 mL) was stirred at room temperature for 10 min. under N₂. Compound 1 (2.0 mmol) was added at once to the above contents and refluxed the reaction over night under N₂. The solvent was removed under reduced pressure and the crude reaction mixture was added to ice cold water (50 mL) with stirring. Filtered the separated solid, washed with water and dried to get pure compound 2.

### Method B

Compound 3 (1.2 mmol) was added at once to the stirred solution of sodium hydroxide (3.6 mmol) in MeOH (10 mL) and stirring was continued for further 10 min at room temperature. R₅CH₂Cl (6.0 mmol)/R₅CH₂Br (1.2 mmol) was added to the above reaction mixture and continue the reaction for 3-5 h under N₂. The reaction mixture was poured into ice water (50 mL), filtered the separated solid, washed with water (10 mL), ether (10 mL) and dried to get pure product 2.

### Example 34

### Methyl 3-amino-8-fluorothieno[3,2-c]quinoline-2-carboxylate (2a)

The reaction of 1a (200 mg, 0.97 mmol) with methyl thioglycolate (104 μL, 1.16 mmol) in the presence of an. K₂CO₃ (535 mg, 3.87 mmol) in dry acetone (10 mL) according to method A resulted in the formation of 2a.

Yield: 260 mg; m.p.: 270-272° C.

¹H NMR (DMSO-d₆; 600 MHz): 9.49 (s, 1H, Ar—H), 8.21-8.19 (m, 1H, Ar—H), 7.98-7.96 (m, 1H, Ar—H), 7.75-7.72 (m, 1H, Ar—H), 7.48 (brs, 2H, NH₂), 3.86 (s, 3H, CO₂CH₃).

LC-MS: (M+H): 277.35.

### Example 35

### Methyl 3-amino-7,8-difluorothieno[3,2-c]quinoline-2-carboxylate (2b)

The cyclo condensation of 1b (200 mg, 0.89 mmol) with methyl thioglycolate (95 μL, 1.07 mmol) in the presence of an. K₂CO₃ (492 mg, 3.56 mmol) in dry acetone (20 mL) followed by purification with column chromatography (Silica gel; Hexane/Ethyl acetate) by following method A resulted in the formation of 2b.

Yield: 160 mg; m.p.: 298-300° C.

¹H NMR (DMSO-d₆; 600 MHz): 9.52 (s, 1H, Ar—H), 8.33-8.30 (m, 1H, Ar—H), 8.19-8.16 (m, 1H, Ar—H), 7.49 (brs, 2H, NH₂), 3.86 (s, 3H, CO₂CH₃).

LC-MS: (M+H): 295.11.

### Example 36

### 3-Amino-8-fluorothieno[3,2-c]quinoline-2-carbonitrile (2c)

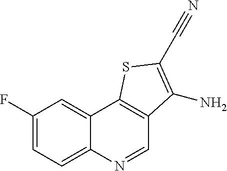 [SMILES: N#Cc1sc2c(cnc3ccc(F)cc32)c1N]

The reaction of 3a (200 mg, 0.98 mmol) with chloroacetonitrile (309 μL, 4.9 mmol) in the presence of NaOH (117 mg, 2.9 mmol) in MeOH (10 mL) gave pure 2c according to the method B.

Yield: 260 mg; m.p.: >300° C.

¹H NMR (DMSO-d₆; 600 MHz): 9.48 (s, 1H, Ar—H), 8.24-8.21 (m, 1H, Ar—H), 8.02-8.01 (m, 1H, Ar—H), 7.78-7.76 (m, 1H, Ar—H), 7.57 (brs, 2H, NH₂).

LC-MS: (M+H): 244.29.

### Example 37

### (3-Amino-8-fluorothieno[3,2-c]quinolin-2-yl)(4-fluorophenyl)methanone (2d)

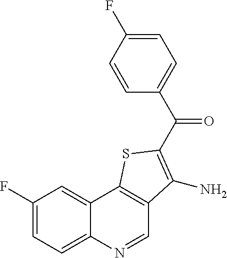 [SMILES: Nc1c(C(=O)c2ccc(F)cc2)sc2c1cnc1ccc(F)cc12]

The reaction of 3a (100 mg, 0.49 mmol) with 4-fluorophenacyl bromide (106 mg, 0.49 mmol) in the presence of NaOH (59 mg, 1.47 mmol) in MeOH (5 mL) gave pure 2d according to method B.

Yield: 150 mg; m.p.: 276-278° C.

¹H NMR (DMSO-d₆; 600 MHz): 9.61 (s, 1H, Ar—H), 8.55 (brs, 2H, NH₂), 8.24-8.22 (m, 1H, Ar—H), 8.02-8.00 (m, 1H, Ar—H), 7.95-7.93 (m, 2H, Ar—H), 7.79-7.75 (m, 1H, Ar—H), 7.45-7.43 (m, 2H, Ar—H).

LC-MS: (M+H): 341.30

### Example 38

### 3-Amino-7,8-difluorothieno[3,2-c]quinoline-2-carbonitrile (2e)

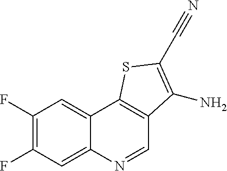 [SMILES: N#Cc1sc2c(cnc3cc(F)c(F)cc32)c1N]

The reaction of 3b (200 mg, 0.9 mmol) with chloroacetonitrile (284 μL, 4.5 mmol) in the presence of NaOH (108 mg, 2.7 mmol) in MeOH (10 mL) by following method B resulted in the formation of pure 2e.

Yield: 225 mg; m.p.: 300-301° C.

¹H NMR (DMSO-d₆; 600 MHz): 9.45 (s, 1H, Ar—H), 8.33-8.30 (m, 1H, Ar—H), 8.18-8.15 (m, 1H, Ar—H), 7.54 (brs, 2H, NH₂).

LC-MS: (M+H): 262.13.

### Example 39

### (3-Amino-7,8-difluorothieno[3,2-c]quinolin-2-yl)(4-fluorophenyl)methanone (2f)

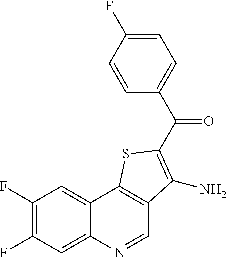 [SMILES: Nc1c(C(=O)c2ccc(F)cc2)sc2c1cnc1cc(F)c(F)cc12]

The title compound 2f prepared by the reaction of 3b (100 mg, 0.45 mmol) with 4-fluorophenacyl bromide (98 mg, 0.45 mmol) in the presence of NaOH (54 mg, 1.35 mmol) in MeOH (5 mL) according to method B.

Yield: 140 mg; m.p.: >320° C.

¹H NMR (DMSO-d₆; 600 MHz): 9.59 (s, 1H, Ar—H), 8.53 (brs, 2H, NH₂), 8.29-8.26 (m, 1H, Ar—H), 8.16-8.13 (m, 1H, Ar—H), 7.93-7.90 (m, 2H, Ar—H), 7.44-7.42 (m, 2H, Ar—H).

LC-MS: (M+H): 359.22.

### Example 40

### (3-Amino-7,8-difluorothieno[3,2-c]quinolin-2-yl)(4-methoxyphenyl)methanone (2g)

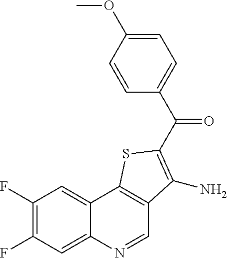 [SMILES: COc1ccc(C(=O)c2sc3c(cnc4cc(F)c(F)cc43)c2N)cc1]

The cyclo condensation of 3b (50 mg, 0.23 mmol) with 4-methoxyphenacyl bromide (51 mg, 0.23 mmol) in the presence of NaOH (27 mg, 0.68 mmol) in MeOH (5 mL) according to method B afforded 2g.

Yield: 64 mg; m.p.: 258-260° C.

¹H NMR (DMSO-d₆; 600 MHz): 9.60 (s, 1H, Ar—H), 8.45 (brs, 2H, NH₂), 8.32-8.29 (m, 1H, Ar—H), 8.18-8.15 (m, 1H, Ar—H), 7.87 (d, 2H, Ar—H, J=12 Hz), 7.13 (d, 2H, Ar—H, J=12 Hz), 3.89 (s, 3H, OCH₃).

LC-MS: (M+H): 371.26.

### Example 41

### (3-Amino-8-fluorothieno[3,2-c]quinolin-2-yl)(4-methoxyphenyl)methanone (2h)

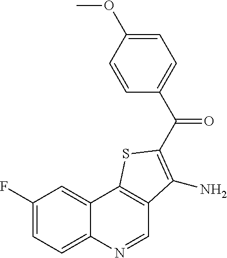 [SMILES: COc1ccc(C(=O)c2sc3c(cnc4ccc(F)cc43)c2N)cc1]

The reaction of 3a (50 mg, 0.25 mmol) with 4-methoxyphenacyl bromide (56 mg, 0.25 mmol) in the presence of NaOH (29 mg, 0.74 mmol) in MeOH (10 mL) according to method B afforded 2h.

Yield: 72 mg; m.p.: 256-258° C.

¹H NMR (DMSO-d₆; 600 MHz): 9.60 (s, 1H, Ar—H), 8.46 (brs, 2H, NH₂), 8.25-8.22 (m, 1H, Ar—H), 8.02-8.00 (m, 1H, Ar—H), 7.89 (d, 2H, Ar—H, J=12 Hz), 7.79-7.75 (m, 1H, Ar—H), 7.14 (d, 2H, Ar—H, J=12 Hz), 3.90 (s, 3H, OCH₃).

LC-MS: (M+H): 353.28.

### Example 42

### Cancer Cell Assays

The effect of the compounds of the invention on tumor cells was determined by the assay described by Latham et al., Oncogene 12:827-837 (1996). Tumor cells K562 (chronic myelogenous leukemia; leukemia cell line +ve for Bcr-Abl) or DU145 (prostate cancer) were plated in 12-well dishes at a cell density of 2.5×10⁴ cells well. The plated cells were treated 24 hours later with a compound of the invention dissolved in DMSO at multiple concentrations ranging from 0.01 μM to 100 μM. The plates were examined 96 hours later under an inverted microscope, Olympus CK-2 using a 10× objective, and compound activity was noted by physical observation. When necessary, the total number of viable cells was determined by trypsinizing the wells and counting the number of viable cells, as determined by trypan blue exclusion, using a hemacytometer. The results of these assays are provided below in Table 1.

TABLE 1

| | K562 | DU145 |
|---|---|---|
| Example # | (μM) | (μM) |
| 1 | 0.15 | 0.6 |
| 2 | >0.5 < 1.0 | >1 |
| 3 | >10 | >10 |
| 4 | >10 | >10 |
| 5 | >1 < 10 | >10 |
| 6 | >0.5 < 1.0 | >1 < 10 |
| 7 | >10 | >10 |
| 8 | >1 < 10 | >10 < 25 |
| 9 | >1 < 10 | >10 |
| 10 | >100 | >100 |
| 11 | >1 < 10 | >1 < 10 |
| 12 | >1 < 5 | >1 < 5 |
| 13 | >10 < 25 | >10 < 25 |
| 14 | >10 < 25 | >10 < 25 |
| 15 | >10 < 25 | >10 < 25 |
| 16 | <1 | <1 |
| 17 | >0.1 < 0.25 | >0.5 < 1.0 |
| 18 | 0.15 | 2.5 |
| 19 | >0.1 < 0.25 | >0.25 < 0.5 |
| 20 | 3 | 25 |
| 21 | <1 | >1 < 10 |
| 22 | >0.1 < 0.5 | >0.5 < 1.0 |
| 23 | 0.2 | 0.75 |
| 24 | ND | ND |
| 25 | >1 < 10 | >1 < 10 |
| 26 | >50 < 100 | >25 < 50 |
| 27 | >1 < 10 | >1 < 10 |
| 28 | >1 < 10 | >10 < 25 |
| 29 | ND | ND |
| 30 | >5 < 10 | >10 |
| 31 | >50 < 100 | >100 |
| 32 | >10 < 25 | >10 < 25 |
| 33 | >25 < 50 | >50 < 100 |
| 34 | 10 | 10 |
| 35 | 10 | 10 |
| 36 | 75 | 75 |
| 37 | 0.15 | 5 |
| 38 | 25 | 25 |
| 39 | 75 | 75 |
| 40 | 75 | 75 |
| 41 | 0.6 | 10 |

### Example 43

### PI3K Isoform Assay

The compound of Example 18, methyl 3-(4-fluorobenzamido)-7,8-dimethoxythieno[3,2-c]quinoline-2-carboxylate, was tested for ability to inhibit the four PI3K isoforms PI3Kα, PI3Kβ, PI3Kδ and PI3Kγ in a homogeneous time-resolved fluorescence (HTRF) enzyme assay.

### A. Assay Description:

The assay relies on the action of the aforesaid kinases on the substrate phosphatidylinositol (4,5)-bisphosphate (PIP2), providing the product phosphatidylinositol (3,4,5)-trisphosphate (PIP3). The PIP3 product is detected by displacement of biotin-PIP3 from an energy transfer complex consisting of Europium labeled anti-GST monoclonal antibody, a GST-tagged pleckstrin homology (PH) domain, biotinylated PIP3 and Streptavidin-Allophycocyanin (APC). Excitation of Europium in the complex results in an energy transfer to the APC and a fluorescent emission at 665 nm. The PIP3 product formed by PI 3-kinase(h) activity displaces biotin-PIP3 from the complex resulting in a loss of energy transfer and thus a decrease in signal.

### B. Enzymes:

The following enzymes were utilized in the assay:
Human PI3Kα (p110α/p85α): Complex of N-terminal GST-tagged recombinant full-length human p110α (GenBank Accession No. U79143), and recombinant full length, human p85α (no tag) (GenBank Accession No. XM_{—}043865), coexpressed in a Baculovirus infected Sf9 cell expression system. p110α MW=155 kDa, p85α MW=83.5 kDa.
Human PI3Kβ (p110β/p85α): Complex of N-terminal 6His-tagged recombinant full-length human p110β (GenBank Accession No. NM_{—}006219), and recombinant full length, human p85α (no tag) (GenBank Accession No. XM_{—}043865), coexpressed in a Baculovirus infected Sf21 cell expression system. p110β MW=124 kDa, p85α MW=83.7 kDa.
Human PI3Kδ (p110δ/p85α): Complex of N-terminal GST tagged recombinant full-length human p110δ (GenBank Accession No. NM_{—}005026), and recombinant full length, human p85α (GenBank Accession No. XM_{—}043865) coexpressed in a Baculovirus infected Sf9 cell expression system. p110δ MW=146 kDa, p85α MW=83.5 kDa.
Human PI3Kγ (p120γ): (GenBank Accession No. AF327656), full length with N-terminal His tag, expressed in a Baculovirus infected SP) cell expression system. MW=131 kDa.

### C. Assay Procedure:

The enzymes (PI3Kα, PI3Kβ, PI3Kδ or PI3Kγ) were delivered into the following kinase reaction buffer containing 10 μM PIP2 substrate: HEPES 50 mM (pH7.0), NaN₃ 0.02%, BSA 0.01%, Orthovanadate 0.1 mM, 1% DMSO. Compounds in 100% DMSO were delivered into the kinase reaction mixture by Acoustic technology (Echo550; nanoliter range), and incubated for 10 min. at room temperature. Next, ATP (10 μM ATP under standard conditions) was delivered into the reaction mixture to initiate the kinase reaction. The mixture was incubated for 30 min. at 30° C., then quenched with a stop solution. A detection buffer (HEPES 10 mM (pH7.0), BSA 0.02%, KF 0.16 M, EDTA 4 mM) was added and the mixture was incubated overnight. Homogeneous time-resolved fluorescence (HTRF) was measured (Ex=320 nm, ratio of Em=615 nm and Em=665 nm). The PI3K inhibitor 2-(4-morpholinyl)-8-phenyl-chromone (LY294002) was included as a positive control. LY294002 is a known inhibitor of all four PI3K isoforms. Compounds were tested in a ten-dose 1050 model with three-fold serial dilutions starting at 20 μM. The emission ratio is converted into μM PIP3 production based on PIP3 standard curves. The nonlinear regressions used to obtain the standard curves and IC₅₀ values were generated with GraphPad Prism software. The results are provided in Table 2:

TABLE 2
PI3K Isoform Assay Data

| | IC50 against | IC50 against | IC50 against | IC50 against |
|---|---|---|---|---|
| Compound | PI3Kα (nM) | PI3Kβ (nM) | PI3Kγ (nM) | PI3Kδ (nM) |
| Ex. 18 | 25340 | 8544 | 1010 | 25750 |
| LY294002 | 316 | 2057 | 896.1 | 319.5 |

The compound of Example 18 inhibits PI3Lγ isoform with an IC₅₀ of 1 μM and does not inhibit other isoforms of PI3K. The same compound was found to inhibit the growth of K562 and DU145 cancer cell lines with a IC₅₀ of 0.15 μM and 2.5 μM, respectively.

All references cited herein are incorporated by reference. The present invention may be embodied in other specific forms without departing from the spirit or essential attributes thereof and, accordingly, reference should be made to the appended claims, rather than to the foregoing specification, as indicating the scope of the invention.

What is claimed is:

1. A compound of Formula I or a salt thereof wherein: 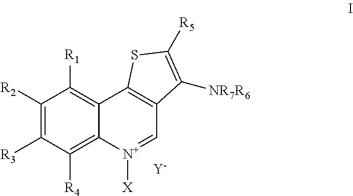
R₁ is selected from the group consisting of: H; halogen; nitro; cyano; —O—R₈; —O—C(═O)—(CH₂)ₙR₉; —C(═O)—R₁₀; —NR₁₁R₁₂; and a saturated or unsaturated, straight or branched, cyclic or acyclic, chiral or achiral hydrocarbyl group having from 1 to 10 carbon atoms, wherein the hydrocarbyl group is optionally substituted with one or more of hydroxy, (C₁-C₄)alkoxy, amino, (C₁-C₄)alkylamino, (C₁-C₄)dialkylamino, halogen, cyano, nitro, carboxy, (C₁-C₄)alkoxycarbonyl or sulfonamido;
R₂ is selected from the group consisting of: H; halogen; nitro; cyano; —O—R₈; —O—C(═O)—(CH₂)ₙR₉; —C(═O)—R₁₀; —NR₁₁R₁₂; and a saturated or unsaturated, straight or branched, cyclic or acyclic, chiral or achiral hydrocarbyl group having from 1 to 10 carbon atoms, wherein the hydrocarbyl group is optionally substituted with one or more of hydroxy, (C₁-C₄)alkoxy, amino, (C₁-C₄)alkylamino, (C₁-C₄)dialkylamino, halogen, cyano, nitro, carboxy, (C₁-C₄)alkoxycarbonyl or sulfonamido;
R₃ is selected from the group consisting of: H; halogen; nitro; cyano; —O—R₈; —O—C(═O)—(CH₂)ₙR₉; —C(═O)—R₁₀; —NR₁₁R₁₂; and a saturated or unsaturated, straight or branched, cyclic or acyclic, chiral or achiral hydrocarbyl group having from 1 to 10 carbon atoms, wherein the hydrocarbyl group is optionally substituted with one or more of hydroxy, (C₁-C₄)alkoxy, amino, (C₁-C₄)alkylamino, (C₁-C₄)dialkylamino, halogen, cyano, nitro, carboxy, (C₁-C₄)alkoxycarbonyl or sulfonamido;
R₄ is selected from the group consisting of: H; halogen; nitro; cyano; —O—R₈; —O—C(═O)—(CH₂)ₙR₉; —C(═O)—R₁₀, —NR₁₁R₁₂; and a saturated or unsaturated, straight or branched, cyclic or acyclic, chiral or achiral hydrocarbyl group having from 1 to 10 carbon atoms, wherein the hydrocarbyl group is optionally substituted with one or more of hydroxy, (C₁-C₄)alkoxy, amino, (C₁-C₄)alkylamino, (C₁-C₄)dialkylamino, halogen, cyano, nitro, carboxy, (C₁-C₄)alkoxycarbonyl or sulfonamido;
R₅ is selected from the group consisting of CN; NO₂; (C₁-C₆)perfluoroalkyl; substituted or unsubstituted (C₆-C₁₀)aryl; substituted or unsubstituted (C₂-C₉)heterocyclyl; substituted or unsubstituted (C₇-C₁₅)aralkyl; substituted or unsubstituted (C₂-C₉)heterocyclylalkyl; and —C(═O)—R₁₃;
R₆ is selected from the group consisting of H; a saturated or unsaturated, straight or branched, cyclic or acyclic, chiral or achiral hydrocarbyl group having from 1 to 10 carbon atoms, wherein the hydrocarbyl group is substituted with (C₁-C₆)carboxy, (C₁-C₆)carboxyalkyl, hydroxy, or (C₁-C₆) alkoxy; and substituted or unsubstituted (C₁₁-C₁₅)aralkyl;
R₇ is selected from the group consisting of H; saturated or unsaturated, straight or branched, cyclic or acyclic, chiral or achiral hydrocarbyl group having from 1 to 10 carbon atoms, wherein the hydrocarbyl group is substituted with (C₁-C₆)carboxy, (C₁-C₆)alkoxycarbonyl, hydroxy, or (C₁-C₆) alkoxy; substituted or unsubstituted (C₇-C₁₅)aralkyl; and —C(═O)—R₁₄;
or
R₆ and R₇ combine to form ═CH—NR₁₇R₁₈;
R₈ is independently selected from the group consisting of:
H;
a saturated or unsaturated, straight or branched, cyclic or acyclic, chiral or achiral hydrocarbyl group having from 1 to 10 carbon atoms, wherein the hydrocarbyl group is optionally substituted with one or more of halogen, hydroxy, (C₁-C₄)alkylcarbonyloxy, or —NR₁₁R₁₂, and wherein one or more of the carbon atoms in the hydrocarbyl group is optionally replaced by an oxygen atom (—O—);
substituted or unsubstituted (C₆-C₁₀)aryl;
substituted or unsubstituted (C₂-C₉)heterocyclyl; and
—NH₂;
R₉ is independently selected from the group consisting of (C₁-C₆)alkyl; substituted or unsubstituted (C₆-C₁₀)aryl; substituted or unsubstituted (C₇-C₁₅)aralkyl; substituted or unsubstituted (C₂-C₉)heterocyclyl; substituted or unsubstituted (C₂-C₉)heterocyclylalkyl; and —NH₂;
R₁₀ is independently selected from the group consisting of —OR₁₅; —NR₁₁R₁₂, and a saturated or unsaturated, straight or branched, cyclic or acyclic, chiral or achiral hydrocarbyl group having from 1 to 10 carbon atoms, wherein the hydrocarbyl group is optionally substituted with one or more of halogens, hydroxy, alkoxy, cyano, carboxy, carbalkoxy, carboxamido, —NR₁₁R₁₂, or nitro;
R₁₁ and R₁₂ are each independently selected from the group consisting of H; (C₂-C₄)acyl; saturated or unsaturated, straight or branched, cyclic or acyclic, chiral or achiral hydrocarbyl group having from 1 to 10 carbon atoms; and substituted or unsubstituted (C₂-C₉)heterocyclyl;
wherein R₁₁ and R₁₂ may combine with O, S, or the nitrogen atom in NR₁₆ to form a substituted or unsubstituted heterocyclyl ring;
R₁₃ is selected from the group consisting of —OR₁₅; and a saturated or unsaturated, straight or branched, cyclic or acyclic, chiral or achiral hydrocarbyl group having from 1 to 10 carbon atoms, wherein the hydrocarbyl group is substituted with one or more of halogens, hydroxy, alkoxy, cyano, carboxy, carbalkoxy, carboxamido, or nitro;
R₁₄ is selected from the group consisting of:
substituted or unsubstituted (C₂-C₉)heterocyclyl;
—C(═O)-substituted or unsubstituted (C₆-C₁₀)aryl;
—C(═O)-substituted or unsubstituted (C₂-C₉)heterocyclyl;
—C(═O)—NH-substituted or unsubstituted (C₆-C₁₀)aryl;
—C(═O)—NH-substituted or unsubstituted (C₆-C₁₀)arylkyl;
—C(═O)—NH-substituted or unsubstituted (C₂-C₉)heterocyclyl;
—C(═O)-substituted or unsubstituted (C₂-C₉)heterocyclyl-substituted or unsubstituted (C₆-C₁₀)heteroaryl; and
—C(═O)—NH-substituted or unsubstituted (C₆-C₁₀)aryl-substituted or unsubstituted (C₂-C₉)heterocyclyl.
R₁₅ is independently selected from the group consisting of (C₁-C₆)alkyl; substituted or unsubstituted (C₇-C₁₅)aralkyl; substituted or unsubstituted (C₂-C₉)heterocyclyl; and substituted or unsubstituted (C₂-C₉)heterocyclylalkyl;
R₁₆ is selected from the group consisting of H; substituted or unsubstituted (C₁-C₆)alkyl; substituted or unsubstituted (C₆-C₁₀)aryl; and substituted or unsubstituted (C₇-C₁₅)aralkyl;
R₁₇ and R₁₈ are each independently (C₁-C₆)alkyl;
n is independently selected from the group consisting of 0, 1, 2, 3, 4, and 5;
X is absent or selected from (C₁-C₆)alkyl and (C₁-C₆)alkoxyoxo(C₁-C₆)alkyl; and
Y is absent or a counterion,
i) provided that when R₆ is H, R₇ is H, and R₁₃ is —OR₁₅, then at least one of R₂ and R₃ is —O—R₈, wherein R₈ is selected from the group consisting of: H; a saturated or unsaturated, straight or branched, cyclic or acyclic, chiral or achiral hydrocarbyl group having from 1 to 10 carbon atoms, wherein the hydrocarbyl group is substituted with one or more of halogen, hydroxy, (C₁-C₄)alkylcarbonyloxy, or —NR₁₁R₁₂; substituted or unsubstituted (C₆-C₁₀)aryl; substituted or unsubstituted (C₂-C₉)heterocyclyl; and —NH₂;
ii) provided that when X is absent, then the N⁺ of Formula I is N and Y is absent, and
iii) when X is (C₁-C₆)alkyl or (C₁-C₆)alkoxyoxo(C₁-C₆)alkyl, then Y is a counterion.

2. A compound according to claim 1, or a salt thereof, wherein at least one of R₁, R₂, R₃, and R₄ is selected from the group consisting of: H; halogen; a saturated or unsaturated, straight or branched, cyclic or acyclic, chiral or achiral hydrocarbyl group having from 1 to 10 carbon atoms, wherein the hydrocarbyl group is optionally substituted with one or more of halogen; and —O—R₈.

3. A compound according to claim 2 or a salt thereof, wherein at least one of R₁, R₂, R₃, and R₄ is H.

4. A compound according to claim 2, wherein at least one of R₁, R₂, R₃, and R₄ is —O—R₈.

5. A compound according to claim 1 or a salt thereof, wherein R₅ is selected from the group consisting of H; CN; —C(═O)—R₁₃; substituted or unsubstituted (C₆-C₁₀)aryl; substituted or unsubstituted (C₇-C₁₅)aralkyl; substituted or unsubstituted (C₂-C₉)heterocyclylalkyl; and substituted or unsubstituted heterocyclyl.

6. A compound according to claim 1 or a salt thereof, wherein R₆ is H.

7. A compound according to claim 1 or a salt thereof, wherein R₇ is selected from the group consisting of H; and —C(═O)—R₁₄; or R₆ and R₇ combine to form ═CH—NR₁₇R₁₈.

8. A compound according to claim 7 or a salt thereof, wherein
R₇ is —C(═O)—R₁₄, and
R₁₄ is selected from the group consisting of:
—C(═O)-substituted or unsubstituted (C₂-C₉)heterocyclyl;
—C(═O)-substituted or unsubstituted (C₂-C₉)heterocyclyl-substituted or unsubstituted (C₆-C₁₀)heteroaryl; and
—C(═O)—NH-substituted or unsubstituted (C₆-C₁₀)aryl-substituted or unsubstituted (C₂-C₉)heterocyclyl.

9. A compound according to claim 1 or a salt thereof, wherein n is 0.

10. A compound according to claim 1, or a salt thereof, selected from the group consisting of:
(1) (3-Amino-7,8-dimethoxythieno[3,2-c]quinolin-2-yl)(4-fluorophenyl)methanone;
(2) Methyl 3-amino-7-benzyloxy-8-methoxythieno[3,2-c]quinolone-2-carboxylate;
(3) 1-(3-Amino-7,8-dimethoxythieno[3,2-c]quinolin-2-yl)-2,2,2-trifluoroethanone;
(4) Methyl 3-amino-8-benzyloxy-7-methoxythieno[3,2-c]quinoline-2-carboxylate;
(5) 3-Amino-7,8-dimethoxythieno[3,2-c]quinoline-2-carbonitrile;
(6) Methyl 3-amino-7-hydroxy-8-methoxythieno[3,2-c]quinoline-2-carboxylate;
(7) Methyl 3-amino-8-hydroxy-7-methoxythieno[3,2-c]quinoline-2-carboxylate;
(8) Methyl 3-amino-8-methoxy-7-(3-morpholinopropoxy)thieno[3,2-c]quinoline-2-carboxylate;
(9) Methyl 3-amino-8-methoxy-7-[3-(piperidin-1-yl)propoxy]thieno[3,2-c]quinoline-2-carboxylate;
(10) Methyl 3-amino-8-methoxy-7-[(4-methylpiperazine-1-carbonyl)oxy]thieno[3,2-c]quinoline-2-carboxylate;
(11) Methyl 3-amino-8-methoxy-7-(2-morpholinoethoxy)thieno[3,2-c]quinoline-2-carboxylate;
(12) Methyl 3-amino-7-methoxy-8-(3-morpholinopropoxy)thieno[3,2-c]quinoline-2-carboxylate;
(13) Methyl 7,8-dimethoxy-3-(4-methoxybenzamido)thieno[3,2-c]quinoline-2-carboxylate;
(14) Methyl 3-(2-fluorobenzamido)-7,8-dimethoxythieno[3,2-c]quinoline-2-carboxylate;
(15) Methyl 3-(4-fluorobenzamido)-7,8-dimethoxythieno[3,2-c]quinoline-2-carboxylate;
(16) Methyl 3-benzamido-7,8-dimethoxythieno[3,2-c]quinoline-2-carboxylate;
(17) Methyl 7,8-dimethoxy-3-(2-nitrobenzamido)thieno[3,2-c]quinoline-2-carboxylate;
(18) Methyl 7,8-dimethoxy-3-(2-aminobenzamido)thieno[3,2-c]quinoline-2-carboxylate;
(19) Methyl 3-(4-chloro-3-nitrobenzamido)-7,8-dimethoxythieno[3,2-c]quinoline-2-carboxylate;
(20) 7,8-dimethoxy-3-(2,2,2-trifluoroacetamido)thieno[3,2-c]quinoline-2-carboxylate;
(21) Methyl 3-(2-chloro-2-oxoacetamido)-7,8-dimethoxythieno[3,2-c]quinoline-2-carboxylate;
(22) Methyl 7,8-dimethoxy-3-[2-(4-methylpiperazin-1-yl)-2-oxoacetamido]thieno[3,2-c]quinoline-2-carboxylate;
(23) Methyl 7,8-dimethoxy-3-(2-morpholino-2-oxoacetamido)thieno[3,2-c]quinoline-2-carboxylate;
(24) Methyl 7,8-dimethoxy-3-{2-oxo-2-[4-(pyridin-4-yl)piperazin-1-yl]acetamido}thieno[3,2-c]quinoline-2-carboxylate;
(25) Methyl 7,8-dimethoxy-3-(2-{[4-(4-methylpiperazin-1-yl)phenyl]amino}-2-oxoacetamido)thieno[3,2-c]quinoline-2-carboxylate;
(26) Methyl 3-(2-chloroacetamido)-7,8-dimethoxythieno[3,2-c]quinoline-2-carboxylate;
(27) 3-amino-7,8-dimethoxy-2-(methoxycarbonyl)-5-methylthieno[3,2-c]quinolin-5-ium iodide;
(28) 3-amino-7,8-dimethoxy-5-(2-methoxy-2-oxoethyl)-2-(methoxycarbonyl)thieno[3,2-c]quinolin-5-ium bromide;
(29) (E)-Methyl 3-{[(dimethylamino)methylene]amino}-7,8-dimethoxythieno[3,2-c]quinoline-2-carboxylate;
(30) 7,8-Dimethoxy-2-(1H-tetrazol-5-yl)thieno[3,2-c]quinolin-3-amine; and
(31) 3-Amino-8-fluorothieno[3,2-c]quinoline-2-carbonitrile;
(32) (3-Amino-8-fluorothieno[3,2-c]quinolin-2-yl)(4-fluorophenyl)methanone;
(33) 3-Amino-7,8-difluorothieno[3,2-c]quinoline-2-carbonitrile;
(34) (3-Amino-7,8-difluorothieno[3,2-c]quinolin-2-yl)(4-fluorophenyl)methanone;
(35) (3-Amino-7,8-difluorothieno[3,2-c]quinolin-2-yl)(4-methoxyphenyl)methanone; and
(36) (3-Amino-8-fluorothieno[3,2-c]quinolin-2-yl)(4-methoxyphenyl)methanone.

11. A compound according to claim 1, or a salt thereof, wherein the compound is methyl 3-(4-fluorobenzamido)-7,8-dimethoxythieno[3,2-c]quinoline-2-carboxylate.

12. A process for preparing a compound of Formula I according to claim 1, said process comprising:
reacting a compound of Formula AA with a compound of Formula BB to produce a compound of Formula Ia, 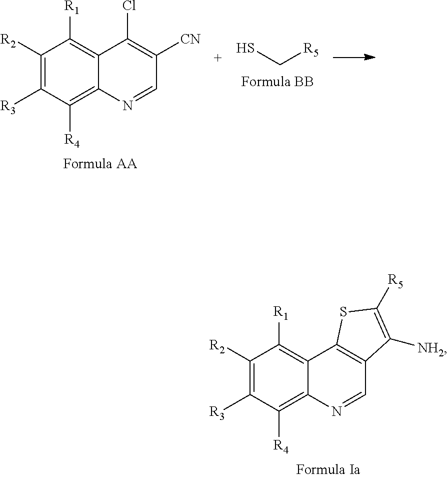
in a reaction mixture, wherein R₁, R₂, R₃, R₄, and R₅ are as defined in claim 1; and optionally, isolating from the reaction mixture the compound of Formula Ia or a salt thereof.

13. A process for preparing a compound of Formula I according to claim 1, said process comprising:
reacting a compound of Formula CC with a compound of Formula DD to form a compound Formula Ib, 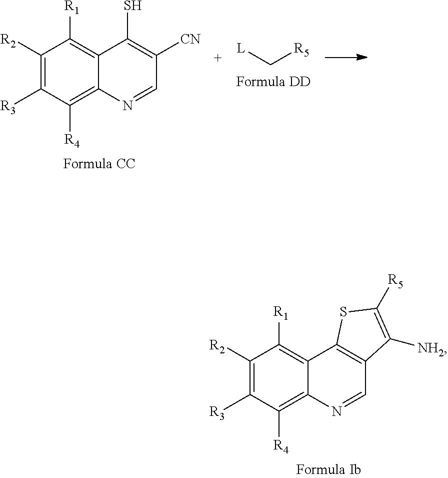
in a reaction mixture, wherein R₁, R₂, R₃, R₄, and R₅ are as defined in claim 1; and optionally, isolating from the reaction mixture the compound of Formula Ib or a salt thereof.

14. The process of claim 13, wherein the aforesaid reaction takes place in the presence of a base.

15. A process for preparing a compound of Formula I according to claim 1, said process comprising a reaction step selected from the group consisting of:
(a) reacting a compound of Formula HH with a compound of Formula II in the presence of a base to form a compound Formula Ie, 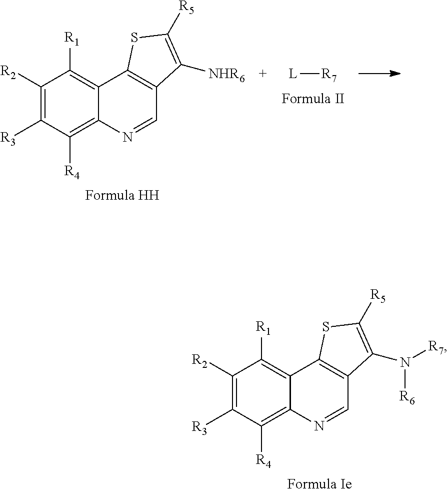
wherein R₁, R₂, R₃, R₄, R₅, R₆, and R₇ are as defined in claim 1 and L is a halogen;
(b) reacting a compound of Formula JJ with a compound of Formula KK to form a compound Formula If, 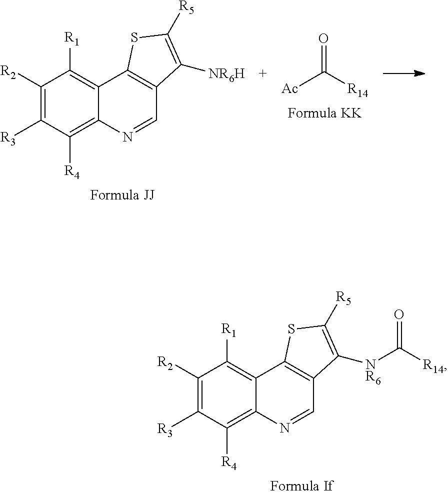
wherein R₁, R₂, R₃, R₄, R₅, R₆, and R₁₄ are as defined in claim 1, and Ac is an acyl group; and
(c) reacting a compound of Formula LL with a compound of Formula MM to form a compound Formula Ig, 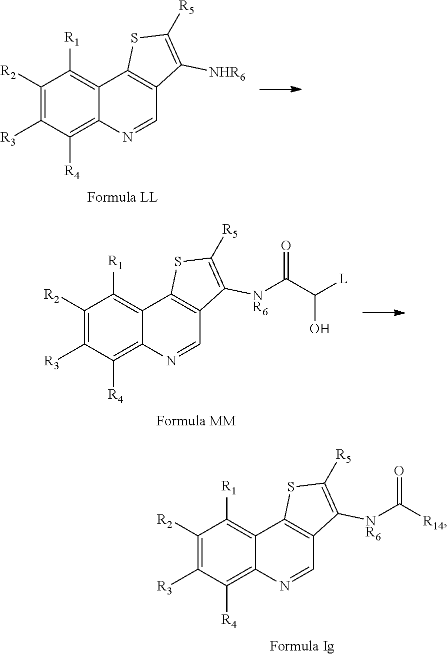
wherein R₁, R₂, R₃, R₄, R₅, R₆, and R₁₄ are as defined in claim 1 and L is a halogen.

16. A process for preparing a compound of Formula I according to claim 1, said process comprising:
condensing a compound of Formula PP with a compound XY to form a compound for Formula Ii 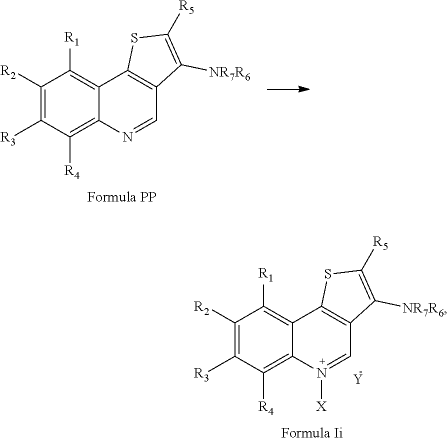
wherein R₁, R₂, R₃, R₄, R₅, R₆, R₇, X and Y are as defined in claim 1.

17. A pharmaceutical composition comprising a pharmaceutically acceptable carrier and a compound, or a pharmaceutically acceptable salt thereof, according to claim 1.

18. A method of treating an individual suffering from a cellular proliferative disorder, comprising administering to the individual an effective amount of at least one compound, or a pharmaceutically acceptable salt thereof, according to claim 1.

19. A method according to claim 18, wherein the compound is methyl 3-(4-fluorobenzamido)-7,8-dimethoxythieno[3,2-c]quinoline-2-carboxylate, or a pharmaceutically acceptable salt thereof.

20. A method according to claim 18, wherein the cellular proliferative disorder is selected from the group consisting of: cancer, malignant and benign tumors, blood vessel proliferative disorders, autoimmune disorders, and fibrotic disorders.

21. The method of claim 18, wherein the cellular proliferative disorder is a cancer selected from the group consisting of: ovarian cancer; cervical cancer; breast cancer; prostate cancer; testicular cancer, lung cancer, renal cancer; colorectal cancer; skin cancer; brain cancer; and leukemia.

22. The method of claim 21, wherein the leukemia is selected from the group consisting of: acute myeloid leukemia, chronic myeloid leukemia, acute lymphoid leukemia, and chronic lymphoid leukemia

23. A method of inducing apoptosis of cancer cells in an individual afflicted with cancer, comprising administering to the individual an effective amount of at least one compound, or a pharmaceutically acceptable salt thereof, according to claim 1.

24. A method of treating an individual suffering from a cellular proliferative disorder, comprising administering to the individual an effective amount of at least one compound of Formula II, or a pharmaceutically acceptable salt thereof wherein: 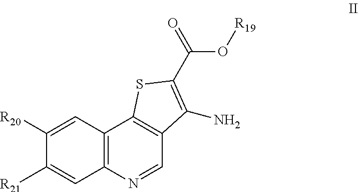
R₁₉ is selected from the group consisting of: (C₁-C₆)alkyl; substituted or unsubstituted (C₇-C₁₅)aralkyl; and substituted or unsubstituted (C₆-C₁₀)aryl; and
R₂₀ and R₂₁ are each independently selected from the group consisting of H, halogen, and —O—CH₃.

25. A method according to claim 24, wherein the compound of Formula II is selected form the group consisting of:
(1-a) methyl 3-amino-7,8-dimethoxythieno[3,2-c]quinolone-2-carboxylate;
(2-a) ethyl 3-amino-7,8-dimethoxythieno[3,2-c]quinolone-2-carboxylate;
(3-a) 1-(3-Amino-7,8-dimethoxythieno[3,2-c]quinolin-2-yl)ethanone;
(4-a) methyl 3-amino-8-fluorothieno[3,2-c]quinoline-2-carboxylate; and
(5-a) methyl 3-amino-7,8-difluorothieno[3,2-c]quinoline-2-carboxylate

26. A method according to claim 24, wherein the cellular proliferative disorder is selected from the group consisting of: cancer, malignant and benign tumors, blood vessel proliferative disorders, autoimmune disorders, and fibrotic disorders.

27. The method of claim 24, wherein the cellular proliferative disorder is a cancer selected from the group consisting of: ovarian cancer; cervical cancer; breast cancer; prostate cancer; testicular cancer; lung cancer; renal cancer; colorectal cancer; skin cancer; brain cancer; and leukemia.

28. The method of claim 27, wherein the leukemia is selected from the group consisting of: acute myeloid leukemia, chronic myeloid leukemia, acute lymphoid leukemia, and chronic lymphoid leukemia.

29. A method of inducing apoptosis of cancer cells in an individual afflicted with cancer, comprising administering to the individual an effective amount of at least one compound of Formula II, or a pharmaceutically acceptable salt thereof, wherein: 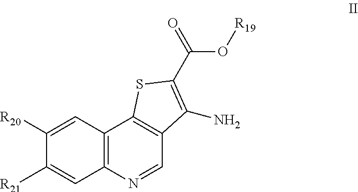
R₁₉ is selected from the group consisting of: (C₁-C₆)alkyl; substituted or unsubstituted (C₇-C₁₅)aralkyl; and substituted or unsubstituted (C₆-C₁₀)aryl; and
R₂₀ and R₂₁ are each independently selected from the group consisting of H, halogen, and —O—CH₃.
